SEQUENCE LISTING (31 sequences)

SEQ ID NO: 1 - DNA, 3501 bp, Homo Sapiens
gcggggctggcaggggcgctgccctggcacagctcggggcctggcagcggcgggtggggcatcggctaagagctgccaccgccgcggggaggggagcccggcccgccgggaccgcaggtaacgggccgcggggccccgcgggccaggaggggaacggggtcgggcgggcgagcagcgggcaggggagctcagggctcggctccgggctctgccgccggatttgggggccgcgaggaagagctgcgagccgagggcctggggccggcgcactcctcccgccctgtctgcagttggaaaacttttccccaagtttggggcggcggagttccgggggagaaggggccgggggagccgcggagggaggcgccgggcccgcgcgtgtagggcccaggccgaggccgggacgcgggtggggcgcaggcccgggtcagggccgcagccggctgtgcgccgtgcccgcccggggcgctgccccctccctcccctgggagctgcgtggctcccccctcccccccacctgcttcctgcctcagcctcctgccccgatataacgccctccccgcgccgggcccggccttcgcgctctgcccgccacggcagccgctgcctccgctccccgcgcggccgccgcccgggccccgaccgagggttgacagcccccggccagggcggcgccagggcgggcaccgcgctcccctcctccgtatcacttcccccaactggggcaacttctcccgaggcgggaggcgctggttcctcggctccctttctccctacttgggtaaagttctccgccctgaatgacttttcctgaagcggacattttacttaaatcgggtaactgtctccaaaagggtcactgcgcctgaacagttttcttctcggaagccccagcacccagccaggtgccctggggcgtgcaggccgccctggcctcccctccaccggcggccgctcacctcctgctccttctcctggtccgggcgggccggcctgggctcccactccagagggcagccggtccttcgccggtgcccaggccgcagggctgatgcccccgctcagctgagggaaggggaagtggaggggagaagtgccgggctggggccaggcggccagggcgccgcacggctctcacccggccggtgtgtgtccccgcaggagagtgtgctgggcagacgatgctggacacgatggaggcgcccggccactccaggcagctgctgctgcagctcaacaaccagcgcaccaagggcttcttgtgcgacgtgatcatcgtggtgcagaacgccctcttccgcgcgcacaagaacgtgctggcggccagcagcgcctacctcaagtccctggtggtgcatgacaacctgctcaacctggaccatgacatggtgagcccggccgtgttccgcctggtgctggacttcatctacaccggccgcctggctgacggcgcagaggcggctgcggccgcggccgtggccccgggggctgagccgagcctgggcgccgtgctggccgccgccagctacctgcagatccccgacctcgtggcgctgtgcaagaaacgcctcaagcgccacggcaagtactgccacctgcggggcggcggcggcggcggcggcggctacgcgccctatggtcggccgggccggggcctgcgggccgccacgccggtcatccaggcctgctacccgtccccagtcgggcctccgccgccgcctgccgcggagccgccctcgggcccagaggccgcggtcaacacgcactgcgccgagctgtacgcgtcgggacccggcccggccgccgcactctgtgcctcggagcgccgctgctcccctctttgtggcctggacctgtccaagaagagcccgccgggctccgcggcgccagagcggccgctggctgagcgcgagctgcccccgcgcccggacagccctcccagcgccggccccgccgcctacaaggagccgcctctcgccctgccgtcgctgccgccgctgcccttccagaagctggaggaggccgcaccgccttccgacccatttcgcggcggcagcggcagcccgggacccgagccccccggccgccccgacgggcctagtctcctctatcgctggatgaagcacgagccgggcctgggtagctatggcgacgagctgggccgggagcgcggctcccccagcgagcgctgcgaagagcgtggtggggacgcggccgtctcgcccggggggcccccgctcggcctggcgccgccgccgcgctaccctggcagcctggacgggcccggcgcgggcggcgacggcgacgactacaagagcagcagcgaggagaccggtagcagcgaggaccccagcccgcctggcggccacctcgagggctacccatgcccgcacctggcctatggcgagcccgagagcttcggtgacaacctgtacgtgtgcattccgtgcggcaagggcttccccagctctgagcagctgaacgcgcacgtggaggctcacgtggaggaggaggaagcgctgtacggcagggccgaggcggccgaagtggccgctggggccgccggcctagggcccccttttggaggcggcggggacaaggtcgccggggctccgggtggcctgggagagctgctgcggccctaccgctgcgcgtcgtgcgacaagagctacaaggacccggccacgctgcggcagcacgagaagacgcactggctgacccggccctacccatgcaccatctgcgggaagaagttcacgcagcgtgggaccatgacgcgccacatgcgcagccacctgggcctcaagcccttcgcgtgcgacgcgtgcggcatgcggttcacgcgccagtaccgcctcacggagcacatgcgcatccactcgggcgagaagccctacgagtgccaggtgtgcggcggcaagttcgcacagcaacgcaacctcatcagccacatgaagatgcacgccgtggggggcgcggccggcgcggccggggcgctggcgggcttgggggggctccccggcgtccccggccccgacggcaagggcaagctcgacttccccgagggcgtctttgctgtggctcgcctcacggccgagcagctgagcctgaagcagcaggacaaggcggccgcggccgagctgctggcgcagaccacgcacttcctgcacgaccccaaggtggcgctggagagcctctacccgctggccaagttcacggccgagctgggcctcagccccgacaaggcggccgaggtgctgagccagggcgctcacctggcggccgggcccgacggccggaccatcgaccgtttctctcccacctagagcgcccctcgccagcccgctctgtcgctgctgcgcggccctggcccgcaccccagggagcggcgggggcggcgcgcagggcccactgtgcccgggacaaccgcagcgtcgccacagtggcggctccacctctcggcggcctcacctggcctcactgcttcgtgccttagctcgg

SEQ ID NO: 2 - DNA, 2501 bp, Homo Sapiens
tttccatagtgtaaatgtgttcccaccactctctggagtaatcctacttaaaaccgttttcagcacaaaattcaaacatctaaacatgatcttgctggctttgcttttgtggctttaccctctttctccccaaacctagctagtgtttgtgctgcctgtaatgcccttctttctttgcaggggtcgccactttaggtcctggtcctccttcagaaagtttttcctctttctccccagcggggatagggtctgtttattttgacaccattagctcacttacacacattggtcacaagtctaggctgcaccgttattgaaagtttaccatctgactctgagtagcttgaggatcctatcaaaactcaggagatgctcagtaaatgttgattgaactatgactgttctcaacatacaaacgcaagatcatttaggaacacttgtcaaaatgtttttgccccttgagattctattttgggaggtaagcagtgggggtccaggactctgcattttgacagtcccctgatgtttgcatgtagaagtgcagggattattacactgacaaatctttaccatccctaagggggactttccttcccaggggctatctctggaagcccctcaaggataggggccgcatgctgtttctctaggtcagcaactaaacccagaaaacgtttattgagtgaatgatgaaacgacaggtgaatagatgaacgcaaggtgtcgagttaactattcttctacacaagtcctagcagctcccattgcttccagccgcagaaatggcccctggaaggcaagtcttccagcgagtggagtcactcttaactacatttcccaggattccaagggagccgcgcgctctgcgctcatcttcctaccagaaatcggcaagtcactgaccctcgtcccgcccccgccattccccgcctcctcctgtcccgcagtcggcgtccagcggctctgcttgttcgtgtgtgtgtcgttgcaggccttattcatgggctcaccgctgaggttcgacgggcgggtggtactggtcaccggcgcgggggcaggtgagcatgcgaaggttggaggccgcgccccttgctgaggcgcagctggctgctcttttcgggccggcatacgcgcgcagccgcagctgaggtcaccccgctgaggtggtggggaggggaatggttattcttgaggcaccgcatctcttgaggaggaaagagccggaaacacctggtctctcaagcaggtacagcccgcttctccccagcaccccggtgtgggcttcccaaggtcctgcctgagaggagaggccaggctgggctgctgattgcaaaactgggtgaaagttctccctgacccttatctgtgggcatcgattgttactcttcctgcaattaactctcttagatctttgcctagtcttttaaaggactgaaaagccgcgaggggcgggggctggaattcgccccctgaagcgcagagatgtcagctcctgaaaagtcattcggtcgttcagtgtttgtttccctctgtcgtaagattttaagttcgtgagaggaccttctttaaagagggcgtctgataagagcccttccccgttggagtttgtatgcttagcaagtcacaatctgttctcgaaatccactggagtcttggcagaggttgtaagctcaaatgcgcacaggggtcaggcgtatgatggagaaagaaaatgggagtaggatgggcacatctgaggaactggagagcagagaattccgaagtggaccggccagtgggaaagttgcctgtatttcaggagcggcaaaatggaaaattgttatgtgaaatagccccattttttaaagtacaaaaaattaaaacaaaccattcataccaacatagatgctgtgcagtgagattttacattagtttctcaccagtgggtgacctctgtaacctccaagtgcagggatcttgacattatgcacctttgattctccactggtagtaccttatacctggaaaggccctaatgcatgaattatttgagttatatattaaacgttacaaactggaattctgtcaattaattcctatgtactttcatatctgtattgataaagtggcttcttatgctgcctttcagaaaatgctttcagtgttgatgaatagccaagtattttatacccatagctgtctggttatctctgcatgggcatgtatttgggtgtagtcataccttctaaatgtttttaggaaaacattttgtttacactttgcttttattgtaaataatgtattttacaacgcttggtgttttaaatcttttttgacagctcttggataattttcatgcaggaggtccagggattacattctaagacgtttttgccatcgctaaggagactttccttttcaggggctatatctgaaaatcattcaaggatagggactgcttcttttgacaccattagcatacttacacatggtatgcagtacattttacaccagtactcagt

SEQ ID NO: 3 - DNA, 2470 bp, Homo Sapiens
aaagatgattaaaagtttaattgttcatctgaagagttgatttttttattcctgtaataaagggtacttttagcagtctctgctcatcttgcccatccggctctttttgtggttgtgtaaggttataacttctgtgtctcagtaaacttgtgcatgcccatttttttctctgttactaccttttctcttattttgttttattattttgatgtaaaattacctgttaattttatttgaaatgagaaattttaaggttcacattattcaaattctgtcagatccctacctctgtcatatggtttataatgtgctgggtattttcagacctgcttattaaaaagatgtaaaacaaaataatgatcactcctgtggatttttcctttatttttgagatgtctcctttggctgcattacttcttcaccccttgcccattgatcagaggaggggtcttaactatgggtgaaccctatatcttactgaagaggttatgttacatgtatattttcataatataacttacatttacatagtacttttatttttagcatacctttttttattaatcctaataatatcactgtaagttatgttgaagcagattgtaagtgttcatttacaaattgtgaaatgaattaaaatgaaagggcaaagattaaatcatgaccaggcctgaaattaacacacaagactcaatttttttcaaccaaagacttttgtaggtgatccctgcctgcaggactccccttcctcctcagatgtcattggattgtaccaggtttactgtagattctagccgttgtagaactaactagatctaagatgagtcccctgatttcctttggtagagtcttccaattgctgaactccaatattgtcgtgactagccagtgttacaacctgtctgccttattttgtgtaatggatttcatattacagaggcatttttttaatgtcaagatgtttaagtattgcttaagtgcaaactacttaatactttttagctattaagtaattaagataggcaggattttatttgttccaaaatgatttgacctaaactaaaaagagaatgtggatctcctgaatcttacttggttaatcttaatataactcctagcattctataattcttcctaaagtcctcttacctggctatcttttgtatcttctttgtctctcctcttctttcccagtcataataactgccagactctgcttcatttctctttgacagtctctactcctaaggtcatccattctctttaggtatcttttggcctcagtttgagcacagcagatcccaagaccacatatgccatagcataggctattatagtcaaccttttgaataaatgtgattgaactttatgttagtaattcttatttaccatcttcctatcaaaaaggcttaaagtcttcatttaatgctctccttcatgtccattttgttaaatgattgccttttaatgacatcttagaacttcagaactatttcaccatggaggatgtgtaagattagccttttatcaaataaaaagtgtgaaatggaatatgtaatctcattaatccattctggctctaaaattctgtgactatcagataaaattcagaaataaaatagtattactaatataaataaatttttatcataattatatttcctaagttttgcctgtaagaatgggtaaaatatctttaaaaccttgaagaaattattacttgatagaaagtttaatccatctgtgagaaggcaaatgtattcagacacaactaaagttctctcttctattttaatttcatttatcttgaactaagactccactgtttcatcctcttagatgctgctacttgaacaatattgttttgagaccaaaaactagcatattaacacaattcttcttaaacgtcttaagagttttgtttcctttacccctttctttaaaaacaagcagccactaaattttttagtagtgaatttcaaaatcctttttaaccttataggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggtatcaagactgtgacttttaattgtagtttatccatttttattcagtattccctcttgtaaacttgaggtaagacactttacttaaaagtgtattttaaattaagcaataatatgtaaactctttcttgcaaaagttagcatttatatttttaaataagatatattgaattcattcagtgaatcatataaagaaaataagtgtaaaactccaatggctagttagttcttagttctttttaagattaaagagaagagaccaaatatagcatcactgtactgaggcaaggttttctgtgtagttcatagaaactag

SEQ ID NO: 4 - DNA, 7001 bp, Homo Sapiens
aatgcaatggaaaaagagagattgtaaagctagaaggcttaggaattgcctcttgattaggtgtggaaggcaagggaaaatcagccctcgaagaagacagtgagattttaatctgggtggctggagagacagtgatgctgggcacagacacggggaagttgagaggaacaccatgtttgagaatggtgactcatatttgaacaagcctgcaatgcccagcagaccgctggaaaagtggggctggagacacattcaacggaggagccagatcaatctttacccttcttcacctgagagagccagtaagtcacggctggaacgtgtgtgtccagcaggagagggtagggagggaagccaagagagctgggagcccgagtgaagtttttgccaaaggcagaagaggaaagtcggcgtagcacagtatactttcccacccatgctcaccaagcccagggacaaggctcaccaagatgagtttggaagagaatgctggagagaaagtggttaagaaaactgcctttactgaacttcttgggctaactttgattgtaagtctctgaacaatcaaagcctgtgaggagacagctaaccttcttattcttcctatgtcaatagtgaacaattgcagatcccctttcctttccttctcctttcccctgttcctctctcctccctccctgaatactcttgcttttttctgggactggtctagagcatgggtggccattgttgacctacaggaggcaccactgtcaccaacaaagggtaacagtctttcttttcaatatttatttatatccagtatttattttcaatactgactatggagagagctctcctgtgctcaaacactgcaatactgggggtctttcaaagcacaaaaacatatatttgcatgatggcatcattaacatttttatggctttctatttcttttttgtactggtctcaagagccactcataaatctctcagtaactgcatagtgtcccagggccagagaccggccactcctggcattgtgattagagtcatttaatatccaaggtggtgactaatgtctggcaacaaagcctccattgggtgtcatgtgtcctgggaccctgagcgtgggcactctaggagcacctcagtattgcgtgttagtactatggccgagagaatagttgagaaagtggtcaagaggtggatccatgtgaacgccactgggaaatgagagacctcgttcccaatcacggtcagtgcaactcgaaagcctaaaatcagtttaaaacaaaggtatctacctttatcttatgttcatatcctaggcttttaataatacgtatttttcacatgtttacagaaagcagtcaactgagctattcatggaaaggtttgtgggtttggttaacgaagtggaggagtattacatttcagctggaaacacatccctagaatgccaaaacatttattccaaagtctggtttcctggtgcaatcggaggcatggcaatgcctctgttcagagactgggggctagggccagtaaggcatttgatccacatgtatcccagaaggcttttattgttaaattatattctttcggaaaaaccacccatgtcctattttgtaaacttgatatccatacacttttgactggcattctattttagccgtaagactatgattcacagcaagcctgtttttcctcttgcttggggtggcagcagaaagcatagggtactttccagcctccaagggtaggggcaaaggggctggggtttctcctccccagtacagctttctctggctgtgccacactgctccctgtgagcagacagcaagtctcccctcactccccactgccattcatccagcgctgtgcagtagcccagctgcgtgtctgccgggaggggctgccaagtgccctgcctactggctgcttcccgaatccctgccattccacgcacaaacacatccacacactctctctgcctagttcacacactgagccactcgcacatgcgagcacattccttccttccttctcactctctcggcccttgacttctacaagcccatggaacatttctggaaagacgttcttgatccagcagggtaggcttgttttgatttctctctctgtagctttagcattttgagaaagcaacttacctttctggctagtgtctgtatcctagcagggagatgaggattgctgttctccatgggggtatgtgtgtgtctcctttttctttcaggacttgtaggattctttgtgccatttgcatataatttggcaggttcacattttttaagagccctatgaagtgctttttgcatgtgttttaaaaaggcatttgaaaattgaaagtgtgatttatggaaattaaatcatctgtaaaaaattgctttggaaagtaatgattgctggccataaagggaaatatctgcgatgcacctaatgtgtttttaaccctttatttgctgacaatctatagtcattaatgctaaactcgattttggcttcagctacatttgcatattgtccaacaatggtctatttttgtaagaattagataaaatgtatacttgatataaaatagtcaaaaatgtaactcttagtaacagtaagcttggcatttagatagaccatgaacacttcgtcagatactctgttgggtgtttgggatagcaattaaaacaaagtattgatagttgtatcagagtctattaggctgcagcaaaggaagtttattcaaaagtataaactatccaagattatagacgcatgatatacttcacctattttttgtctccttaatatgtatatatatatatatatatatatatatacacatatatgtgtgtgtgtatgtgcgtgtgcatgtttaacttttaattcagttaaaaacttttttctatttgtttttcatctggatatttgattctgcatatcctagcccaagtgaaccgagaagatcgagttgtaggactaaaggatagacatgcagaaatgcattttaaaaatctgttagctggaccagaccgacaatgtaacataattgccaaagctttggttcgtgacctgaggttatgtttggtatgaaaaggtcacattttatattcagttttctgaagttttggttgcataaccaacctgtggaaggcatgaacacccatgtgcgccctaaccaaaggtttttctgaatcatccttcacatgagaattcctaatgggaccaagtacagtactgtggtccaacataaacacacaagtcaggctgagagaatctcagaaggttgtggaagggtctatctactttgggagcattttgcagaggaagaaactgaggtcctggcaggttgcattctcctgatggcaaaatgcagctcttcctatatgtataccctgaatctccgcccccttcccctcagatgccccctgtcagttcccccagctgctaaatatagctgtctgtggctggctgcgtatgcaaccgcacaccccattctatctgccctatctcggttacagtgtagtcctccccagggtcatcctatgtacacactacgtatttctagccaacgaggagggggaatcaaacagaaagagagacaaacagagatatatcggagtctggcacggggcacataaggcagcacattagagaaagccggcccctggatccgtctttcgcgtttattttaagcccagtcttccctgggccacctttagcagatcctcgtgcgcccccgccccctggccgtgaaactcagcctctatccagcagcgacgacaagtaaagtaaagttcagggaagctgctctttgggatcgctccaaatcgagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggtacccgcgcccgcgccgcccgtcggggtggccgccgcgcccggcaggagggagggagggagggagggagaagggagagcctagggagctgcgggagccgcgggacgcgcgacccgagggtgcgcgcagggagcccggggcgcgcggcccagcccgggggttctgcgtgcagcccgcgctgcgttcagagtcaagttctctcgccgggcagctgaaaaaaacgtactctccacccacttaccgtccgtgcgagaggcagacccgaaagcccgggcttcctaacaaaacacacgttggaaaaccagacaaagcagcagttatttgtgggggaaaacacctccaggcaaataaacacggggcgctttgagtcacttgggaaggtctcgctcttggcatttaaagttgggggtgtttggagttagcagagctcagcagagttttatttatccttttaatgtttttgtttaatgtgctccccaaatttcctttcatctagactatttgattggaaatatgtcagctatgatgatgactttctgggaagcgattcctgtcacccgctttcccctcctccccaccccacgtcctggggctttagagagcgattgggagttgaatgggtctgatttcggagttagctggctgagtccgcgctggagcggattgctggcatgtgacttctgacagccggaaatttgtaggtgtcccgcgagtttaaaacaagccatatggaagcacaagtgcttaaaaataatctcctgccagcccagtgacaagcctgtcccacccggggagaatgccccggagtggcgtgcgggtcagccagggtctgcgcctcgcagccactgtggaaggagcgcggccggtccaggacacaggagaccactttgtgacttcaatggcgaaggttgtgtgtcctcattttaatttttttccctacaagaattgttctttctccctctcctctccctcccattttctcttgcccagtttctccttttgttttttgttttttgttttcctgatgggcctgcagagggattaggtgggcgcttctggtgaacaccttcctaggtggccacaggacaggtgtaccccggactgggtttggaagcttcagggcgccacatggctgggtcctgaattaggcatttcccaactgtacactggtatccggactggtgtccctatatctttctgccttgtaagccgtggaccagtttttgttcagtattctgtttccagggatatttatagcagaaggaaggggactaaagtgcagtttggccccagaggatactgaagggcagattctgggggtattcagtgtgcatcttcagccgccttggagaaatttagagcatcccacagccacgcagatccaagctgtctttactcaaaagacaaacaatgaacaaaacttttaaaggttggcatatttcaaattaattttacttgttttaatttagggttaaaacagagaaaaaggatttcttctgcccacctttttttttttaaatggaagaacaaagtacagcgattaagtctaattccacacaacatttaaaactgcttgatgtgaaggaaggcactggtatgatgtgaattccataaccttatgatggactccagaaaccattttcttccctatttaattttcagttcttttattgcaaattaatgctgctgaatttcaatgggcactaatgagactgctccttggtagattatttactgccttgctaataattacaaagtgaacctggtcaaatacagaggggatcgcatcttattcaaaattgttcatcatcccagtgataagtggtatcagtgtaatatgccctatcttacactttctgcattacatgatattcaaacactcttagaataataaaaaaagagacaaggaacttaaaaattaaaaaaaaaacttgcacaaatgggactctgtgtggaaattcagttttagaatgatttttcctgtgttttatttcccggattatctttcctcttttgttagaattctgcctgttattatccagcaaggaaaagaagcatctatgcaagttcttcatatggacagatattatttagtatttttcccctctcagtttttctgcttaaatgactctgggtataaaggaaaggattgattgggctcttttaggaaactttaagtttcttaagtagttctcaaaagttttggggctgaaagcagtgttttcaaactgcttgtcatgacccagagggtcatgaactcagtttagtgagtctagaatattttttaaaaggactaaaatggaaaggaatataatagaaaatatcagagtgcatggtatttcgtaaggataagttttgt

SEQ ID NO: 5 - DNA, 11001 bp, Homo Sapiens
ccaagtcagatgttccccaattacctgtggacaggtcaggcatattctgagtctaatttcactccacaggcctaataccttggagccagaaagcttccaggtaaaaagtctgaagggggcctcctcatgtcattagatggactcctgcatctccagaagattttccacaccaggaaagatcaaagcaccaaggcaattcttcctggcttcttgggacaaccctaggctttggcatgagtggtctggaagcctttgctttagttacaatgcctatacactcctggaactgttttgcagggcttgtcttccagcacaattcctcctccaagccttactgtagctacagcccatcagtcctgtctagtgacaaccaagaaactaagaactatgtactcacgttcacctccccagagtcattttccttcaggacaaagctcagggccttgtcactgggccctgccaggagccgcagccgcaggggctgctcatcatccaacagcttccgcaagtacactgtgaaggggaagtaatgatcagagacagggccagctgctcagcccctgcatgctcaggtgcatgcgtatataccctcacatagggcagggtggggtgggaagcccaccttggccgtgacgctcagcgcgctcaaagagtgcaaacttgcgggggtcatccaccaccaagaactttcgcagcagggcctcaatgacttcacgtgcccttgtgcgtgacagcacatgcaggtgcttgacagcatccttgggcaggtaaaaggaagtgcggcgcctgacacttgtgccccgtcctgggccccgccgggcatcctgcaaggagggtggcttcttgctggagggcacagagacagggcgcaccagcttcagctgaaccttgatgaagcctgtgtaagaaccgtccttgttctaaagaaatagagaaaccaaaccttgataataggttccaggtgagatgtcagtctacttggggctaggctgggtatgcacaaattactgcttgcgcccacccaagataacctcagttgtgaccctctgagtatcaggcacatagctgggtacctgctcctccccacgcccccttcctgagcagtcaactcaccaagctcatgaagaggttgctgttgatctgggcattgtactccttgatcttctgctcaatctcagcttgagaaaggtcaggtgtctcccactccacaggctcgtcctgcaagatgggccagcatggacacagggcccttgaggaacccagggcttctctgaaaaatggcctctggggcagtctttggaaactgactgcctttggccccctgtccctgatgtacatatacatagctggtgcccaccctgaacccaccactgctcctggttttgcatgctctgggtggataagggaaagacagaatcatttggcttctctctgctgcctgcctagggcctcagcactgaatgtagccttaaggataccacagaagcaggggcaactgaaggcacatggccaggggccaggaacagctgagggactctgaagagggactctcatttaaagtaaaatcaggctgggtgtggtggctcacatctgcaatcccagcattttgggaggctaaggtaggaggatcacttgatcctcaggagtttgagaccagcttgggcaacatagcaagacctcatctctactaaaaaaagaaaaaaaaaaattagccaggtgtggtggtgtgcctgtagtcccaactgttcaggaggctgaggtgggaggatcgtttgagcccgggagattgcagctacagtaagctattatcgtgtcactgcactccagcctggggaactgagtgagaccctgcttcaaaacacaaaaaacaaaaacaggctgggcacgttggctcacgcctgtaattctagcactttgggaggccgaggcgggtggatcacctgaggtcaggagtttgagaccagcctgaccaacatggagaaaccccgtctctactaaaaatacaaaattagccaggcgtggtggcacatgcctgtaatcccagctacttaggaggctgaggcaggagaattgctcaaactcgggaggtggaggttgcagtgaactgagatcgtgccatcgcactccagcctgggcaacaagagcgaaactcggtctcaaaaaaaaaaaaaatcagtaaaatcacacctcaattgcacattctgatcacagcaccctagttgagttggagtgagggtttgtcctggagaaggcagcccatttttctcctctgccccggcacggggccatgacccactgcagggtgagaggagtggagagtggtgcacatcagtagtccagccaccagtggacagagtagtacttggagccagttctccatgtctcacacatagtgagaaaaatcactgtgacatgatgtttaaccttgacccaagctgcataaaaggcagctttaggccaggctccaatctgccagaggtacacaggcagcttcctggtgggtttctgcacctgcctgtgctgtctggagatttggcccaaagattttttttttttttgagacgaagcctcactctgtcgcccaggctgtagtgcagtggctggatcttggctcactgcaagttctgcctcctgggttcaagcgattctcctgcctcagactcccgagtagctgggactacaggcgcgtgccacaacaacacccggctaatttttgtatttttagtagagatgggatttcaccacattggccaggttggtcttgaactcctgacctcaagtgatccgcctgccttcacctcccaaagtgctgggattacaggcgtgaaccatcgtacccgacccagagatttttaactcgaccactcactccccacctcatctagggactggattcttgccggaagggtggagtgtgggacagggcagccagggctctgaaccgactttcttctcccagactcccttggccccactgcatcagccttacttcctgttgacgtcagataggccctagttagaatgcgagtgtcacagacacagctaagctcagcgctgaccaatactttgtcccagaagaattcccacaaggtttcctgtagaatgatcttgtgcctagcccaggagagccagggttctccctgactccgccctggagtccccttaagcacttaaaccatctgatggggacaaatggagaggacagatgagggagcagggtggagcgttttagcagaatgctccttacccagaacccgctgctattctgcagccagcaaggatgtggggctaagaactaaggccagggccttacaggaaaaaggtaaagggggaggggtgggaatttaagctcattttcttccccaagtatccaaaggtctcctggatggagaagagcactggagtaaaaaccccagtacaaaccttactggggacagtgggcaaccttgtcgggttagtaaaaacaaatggtgtgggccctggaaaatgagggctggaggctgtgaataaagcagtggatgtgtttgttcagtacaccaacgggaagaagtacccagatgggaggagtactaggggcaggagaaatgccagacagactctagtgccagggcaagaaggaagatcattttgtttgcagaacagggagggcacagggatggtgctaacttgttcttgtgatggctctgagctcctacctaacaatgagaaagcttgctccttcttcccttcctggatgacccaggagccctgggctgggatgcagtgacctcatttccagccccttcccttctggtgatgaacctccctatcttcactcagaaaacagacttggattagaggcactgcacagcccttccaggattctaaaggaggaagagtttctttttctgtttccaaagctgcctgctggaagaggatttcaacagccatcccagtcggatgcacagcaggaccatggaatttcccttctgcaccatagggacccaccctccactctaccactgtccataaaaactgatggttttttttttgagacagagtctcgctctgttttccaggctggagtgcagtggtgcgatcttggctcattgcaatctctgcctcctgggttcaagcaattctctgcttcagcctcccaagtagctgggattacaggtgcctgccaccacaactggctaattttttgtatttttagtcgagacggggtttcaccattttggccaggctggtcttgaactcctgacctcatgatccacccacctcggctttccaaagtgctgggattaaaggtgtgagccactgcacctggcctaaaactgatgtttttttctttttttttaacatataacttgggacttctcagcctcctattctttctttttttttttttttttttttgagacagagtcttgctctctcatccaggctggaatgcagtggcccagtctcgactcactgcaacctctgtcttctgggttcaagtgatactcctgcctcagcctccccagtagctgggattacaggcacacaccaccatggccagataatttttttgtattttcagtacagacggggttttgctatgttggcctggcaggtctcgaactcttggcctcaagtgatctgcctgccttggcctcccaaaatgctgagattacaggcatgagtcaccaagcccagccttctttcttttttttgagacagagcctcaccctgtcacccaggttggagtgcagtggcacgatcttggctcactgcaacctttgcctcccggttgaagtgattcagtctcccaagtagctgggactacagtcacacaccaccatgcccggctaatttttgtatgtttagtagagatagggtttcaccatgttggccaggctgacctcgaattcctgattgcaaatgatccacctgccttggcctcccaaagcattggcattagaggtgtgagccaccgtacttggcttccttttctatttttgagacagagtctcactctgtcactcaggctggagtgcagtggcacgatcttggctcactgcaacctctgcctcccaggttcaagtgatccttctgcctcaccctcccaagtagctgggattacaggtgtgcacctccgtggctagccctccttttcaattggttagtgtcttgtggttttcccacctttccacagtggaaaatggctcaggactgactgacatgaagacaagcccaggggtctacactcaactcaacccttgcacccaagctctgggctaagattttggcgtgctgagcaccacccattttgtaaggaattttgtaaaattttatctgaagcatcactcacaactccactttctttacttaaataaggatttccgccccatttctgccaggcatactgagcttcacagtccctgtttctttttcctggtgcctaggcctggttctctgagcctggtggtcacaccaatggcatctggcacacagttctccgataatggggatacctaggaggttccgagacaccttacagtcctgggttagtaacctggatctctttttccacctctttaggcattttataatctagctttcccccttcctgtgggtaaagtgctcctgaatgcttatggtccaaaacaagacttctttcctatctattcccaaatctttctccagatccaccctagaggaagggaacagaatcttccacattccagcagctggtgacaggccagaacagggaagaggtgagggctcagctggctccatacaggagtgcagatggaggagcaggatctctctctgcctctcaagttttcctaaacatacttctcaattcctggcgaggactcttccctctccacatcctcccctagtctccccaaggagggagcaggagcattcgaacgcggaaatcgaggtgctagtccaaactgctcggtcggctttagtcatagctggataatgcccggctcaggtctaccacaagccatacagctgctttttccgtgttcaacctgtctgtgacagaaaccaagggggccccggcacccagcatctaggcggtggaatcggggtcttacgcacggttccgcgggcaggtccccggccaggacccgcggggagccacgtagccaggagggtggggctgcccaccgacccaggacgcggcaacggaccggggagggcggagctccagcgaccgcttcccctcccgcccgccggcaccccctggctcccacctggtcccggcgcggcctgcgagctagcgaggttcgcgcggtgaagtactgctcgagctccgagtccgagtcctcttggctgcagtagccactgctcgtcgtgctgctccaggtcatttcgaaagaaggcgcctccgcctcgcccatagccgtacccgcccgtcccccagtcctgcgcgtccgtagccgccaaccaccgccccggtcgcgtgcgtgcgtgtacgcgtgtcagtgtgcgcgtgcgcccgggccagagccgcgccgcaaccgttaagactgaaacgtagatcgccgggatctagctcttgtctcattggggcaggaacgccggggcggggacacgcacgcttcgcccccaggaatgacctcatcgctccggagctccactcacagaccccacctaccacagggaacgggggcgggtgccagcgtccgggcaagcgcacaagagtggcctctggccggaggcgagggcgggaaggtgcgggaagtgcgcgtgcgcggagcctgggtcagcctgggcccgggtccgcttgcagcgggtggagtacttgcggagccggcaatccaggctcccctcccagcccccgcgcagaattagcctctctgtgccgccgggaaatcggcaattagaacgctccttgcgcgcggcacccaggcagccctcgagaatgcctgcactgtggcctgcccatcctcgcccttcccatacgccctcggccccgcgctcaccacgttcgtgtcccgctccaccgcgggttcccagcccaggtcccggggcccgcaacagtccaggcagacgagcgcgcggcagcggtagtggcaggtgaacttgcaatctgcagagaggcctggcggtgaggcggaggagctccaggtcggggaaatgtcccggagattgaagggaagccccagggagagggccgctgctcgccaggctccgcaggcccgacctatctcagtgggttacctcacactgctacgcggactctaatgttggccacctgggcgtctggaaaccggccggaaggccacaggcagagaggcctgctcaacagttggatctctatcgcctagcacagaacttcccctttcctcattggcaattaaaaaaacaacaacaaaaaactgcgtcttgcttttgtcacccaggctggagtgcaatggcgcgatttcggctcaccgcaacctccgcctcctgggttcaagcgattcttctgcctcagcctcctgagtacctaggattacaggcgcccgccaccatgcccagctaatttttgtatttttagtagagacggggtttcaccatgttagccaggctggtctcaaactcctgatctcaggtgatccacccgcctcggccttccaaagtgctgggactacaggcttgagccaccgcacccggcccttcactgggaacgtatatggaatacatctgcccatttacttgaaggaaaaactaaacacctttaacctacgtctgccctgtggttgtcacctgtctctactcccctcagaccaagacactggtctctatacactctaatccttcgccttcactctcccctctacccactccagccaggcttgcctcttcctccaggaaactgcccgggacagggtcctcagcgatctgtgtactaccaaatggaatccagtgttccattctccattctcaccccctcagcatcatttgaagcttgctccctttgactcccaggggctacactctcccagttttcctcctaccccctgcagctcctgctcagctcctttgcagattctgactcaacttccatatctcacgatgaagtctgggctcagtcctgatcactggcctggtctgtctacattcatctgccccagatccacggctgaaacactgacctaaaccctcagactagatcctccgtgccagtaccttcactaggatgtctaaaagacgtttcaagtgaacatggccaaaatttaattcccttttcttcagcctcactgctacacttgcccagcttcctctttgcagcaaaaatggccactaggctcccagttactggagacaaaagcccaaacttatctttgatttctcccttgtctctacctctgataaacatgcccaaatcatcctgcttcttatctccatggctactttatttctctttgagaacgctgcaatgtcccagccttgttctttttttttttttttttttttttgagacagagtctcactctgtcgccaaggctggagggcagtggcacgatctcggctcactgcaacctccgcctcctgtgttcaagcaattctcccacctcagcctcccgagtagctgggattacaggcacccgccactacgcctggctcatttttttttattttttagtagacatgaggtttcaccatgttggccaggctggtcttgaactcctgacctcaggtgatccacccgcctccgccttccgaagtgctgggattacaggcatgagccaccgcgctcggccccttgttcattctttgcattctgtcacaactttgtgctccccccagctgaatttgtgatgtcctcttgtaccggatgagagggtctccatgcacacacagacctgggacactatccatccacaagttcctaaataggccagagcagtgatgctcaacccagactccatgttacaataatttggggagtttttaaaatttactgatgcctagggtccactcccagcagttgattcaacaggtctgcggtgggatccaggctagcggggaggactgtaaaagcacccctggtgattccagctggtgtctacccaggggagagcaacctttgcttgctggcgattcccaggggtgcagaaggactgctgggtgtgtggctgcgtgcatattttagcatctgattcactgggtcagaaaagggtgtttgctaaataaagactcaacaaaactcctgcttgcagggggcccaccaaaggttctaaatttttccaggctccctcccataggtggtaatttcccttcaccctaaaggttctggagggggtcatgagtgtttgagaagaggcaagcctgggaagatggactccgaggacagtaggcacaaaccctttctcaagaagggccaaggcattttaaagataagaaacttaaaatcagcgtatttttacatataagcagccacctctgctcatctgtggcccagatacgagtggagtgcgacaagggataaaccattttcgcgcactcttcagcgatggggcgaaagtaacggacctagtcctcgggagctgtccccgccgaccccctctgccgcgacttgacccgcggcgactgcgctgccccttggctgccccttccgctctcgtaggcgcgcggggccactactcacgcgcgcactgcaggcctttgcgcacgacgccccagatgaagtcgccacagaggtcgcaccacgtgtgcgtggcgggccccgcgggctggaagcggtggccacggccagggaccagctgccgtgtggggttgcacgcggtgccccgcgcgatgcgcagcgcgttggcacgctccagccgggtgcggcccttcccagcgcgcccagcgggtgccagctcccgcagctcaatgagctcaggctcccccgacatggcccggttgggcccgtgcttcgctggctttgggcgctagcaagcgcgggccgggcggggccacagggcgggccccgacttcagcgcctcccccaggatccagactgggcggcgggaaggagctgaggagagccgcgcaatggaaacctgggtgcagggactgtggggcccgaaggcggggctgggcgcgctctcgcagagccccccccgccttgcccttccttccctccttcgtcccctcctcacaccccaccccggacggccacaacgacggcgaccgcaaagcaccacgcggagatacccgtgtttctggaggccagctttactgtgctagaggaagagggtccccacatccggccctggccctcctggtccggtttgctgaagcaacacacttggcctacccactgggtggggcaggaagtctcgagccttcacttggggtgaggaggagggagatcggtcagcagctttaccgcccgctctgctctccactgcggagactggggctccggcagaggctggaccgtgatcttgaggttcaggggtgcattctgggtggattcccttggcatgggtggtcggccctcagcaactgcagccctcatttggctctgtcaccctgggctgccaggacacaagtctttccatgcttttcccagtgcttgacttggcactccctgcaggcaggtgggtattgaggatggcaatgcatgtgggggatgtgggagtagggcttagaggtccaaggttctaggataccctcacctgcagcaataccactcattctggcatcgtgagcagcgcttagaagcctctgcactgcagtaagcacagcggggccgctctggagccactgcctctagcacatccagcctgtaggtctcagcccacctgggggaaagtcaggaaggtctgactggccctggaaggtgggggcaccccacccacatccatgcctcctgcatcccctccaccctccctgccatttccacaggccttaccttcgcgcctgcagccgcaggtcctgctctgaggggctgaacacatgctggagctggtgcttggcaattgcctgccacttgcctctgttttctcgctccagccgctcccagatttctgggatctagga

SEQ ID NO: 6 - DNA, 3501 bp, Artificial Sequence
  chemically treated genomic DNA (Homo sapiens)
gcggggttggtaggggcgttgttttggtatagttcggggtttggtagcggcgggtggggtatcggttaagagttgttatcgtcgcggggaggggagttcggttcgtcgggatcgtaggtaacgggtcgcggggtttcgcgggttaggaggggaacggggtcgggcgggcgagtagcgggtaggggagtttagggttcggtttcgggttttgtcgtcggatttgggggtcgcgaggaagagttgcgagtcgagggtttggggtcggcgtatttttttcgttttgtttgtagttggaaaatttttttttaagtttggggcggcggagtttcgggggagaaggggtcgggggagtcgcggagggaggcgtcgggttcgcgcgtgtagggtttaggtcgaggtcgggacgcgggtggggcgtaggttcgggttagggtcgtagtcggttgtgcgtcgtgttcgttcggggcgttgtttttttttttttttgggagttgcgtggttttttttttttttttatttgttttttgttttagttttttgtttcgatataacgtttttttcgcgtcgggttcggttttcgcgttttgttcgttacggtagtcgttgttttcgtttttcgcgcggtcgtcgttcgggtttcgatcgagggttgatagttttcggttagggcggcgttagggcgggtatcgcgtttttttttttcgtattattttttttaattggggtaatttttttcgaggcgggaggcgttggtttttcggttttttttttttttatttgggtaaagtttttcgttttgaatgattttttttgaagcggatattttatttaaatcgggtaattgtttttaaaagggttattgcgtttgaatagtttttttttcggaagttttagtatttagttaggtgttttggggcgtgtaggtcgttttggttttttttttatcggcggtcgtttattttttgtttttttttttggttcgggcgggtcggtttgggtttttattttagagggtagtcggtttttcgtcggtgtttaggtcgtagggttgatgttttcgtttagttgagggaaggggaagtggaggggagaagtgtcgggttggggttaggcggttagggcgtcgtacggtttttattcggtcggtgtgtgttttcgtaggagagtgtgttgggtagacgatgttggatacgatggaggcgttcggttattttaggtagttgttgttgtagtttaataattagcgtattaagggttttttgtgcgacgtgattatcgtggtgtagaacgttttttttcgcgcgtataagaacgtgttggcggttagtagcgtttattttaagtttttggtggtgtatgataatttgtttaatttggattatgatatggtgagttcggtcgtgtttcgtttggtgttggattttatttatatcggtcgtttggttgacggcgtagaggcggttgcggtcgcggtcgtggtttcgggggttgagtcgagtttgggcgtcgtgttggtcgtcgttagttatttgtagattttcgatttcgtggcgttgtgtaagaaacgttttaagcgttacggtaagtattgttatttgcggggcggcggcggcggcggcggcggttacgcgttttatggtcggtcgggtcggggtttgcgggtcgttacgtcggttatttaggtttgttattcgtttttagtcgggttttcgtcgtcgtttgtcgcggagtcgttttcgggtttagaggtcgcggttaatacgtattgcgtcgagttgtacgcgtcgggattcggttcggtcgtcgtattttgtgtttcggagcgtcgttgtttttttttttgtggtttggatttgtttaagaagagttcgtcgggtttcgcggcgttagagcggtcgttggttgagcgcgagttgttttcgcgttcggatagtttttttagcgtcggtttcgtcgtttataaggagtcgtttttcgttttgtcgtcgttgtcgtcgttgtttttttagaagttggaggaggtcgtatcgtttttcgatttatttcgcggcggtagcggtagttcgggattcgagtttttcggtcgtttcgacgggtttagttttttttatcgttggatgaagtacgagtcgggtttgggtagttatggcgacgagttgggtcgggagcgcggtttttttagcgagcgttgcgaagagcgtggtggggacgcggtcgtttcgttcggggggttttcgttcggtttggcgtcgtcgtcgcgttattttggtagtttggacgggttcggcgcgggcggcgacggcgacgattataagagtagtagcgaggagatcggtagtagcgaggattttagttcgtttggcggttatttcgagggttatttatgttcgtatttggtttatggcgagttcgagagtttcggtgataatttgtacgtgtgtatttcgtgcggtaagggtttttttagttttgagtagttgaacgcgtacgtggaggtttacgtggaggaggaggaagcgttgtacggtagggtcgaggcggtcgaagtggtcgttggggtcgtcggtttagggtttttttttggaggcggcggggataaggtcgtcggggtttcgggtggtttgggagagttgttgcggttttatcgttgcgcgtcgtgcgataagagttataaggattcggttacgttgcggtagtacgagaagacgtattggttgattcggttttatttatgtattatttgcgggaagaagtttacgtagcgtgggattatgacgcgttatatgcgtagttatttgggttttaagtttttcgcgtgcgacgcgtgcggtatgcggtttacgcgttagtatcgttttacggagtatatgcgtatttattcgggcgagaagttttacgagtgttaggtgtgcggcggtaagttcgtatagtaacgtaattttattagttatatgaagatgtacgtcgtggggggcgcggtcggcgcggtcggggcgttggcgggtttgggggggtttttcggcgttttcggtttcgacggtaagggtaagttcgattttttcgagggcgtttttgttgtggttcgttttacggtcgagtagttgagtttgaagtagtaggataaggcggtcgcggtcgagttgttggcgtagattacgtattttttgtacgattttaaggtggcgttggagagtttttattcgttggttaagtttacggtcgagttgggttttagtttcgataaggcggtcgaggtgttgagttagggcgtttatttggcggtcgggttcgacggtcggattatcgatcgttttttttttatttagagcgtttttcgttagttcgttttgtcgttgttgcgcggttttggttcgtattttagggagcggcgggggcggcgcgtagggtttattgtgttcgggataatcgtagcgtcgttatagtggcggttttatttttcggcggttttatttggttttattgtttcgtgttttagttcgg

SEQ ID NO: 7 - DNA, 3501 bp, Artificial Sequence
  chemically treated genomic DNA (Homo sapiens)
tcgagttaaggtacgaagtagtgaggttaggtgaggtcgtcgagaggtggagtcgttattgtggcgacgttgcggttgtttcgggtatagtgggttttgcgcgtcgttttcgtcgttttttggggtgcgggttagggtcgcgtagtagcgatagagcgggttggcgaggggcgttttaggtgggagagaaacggtcgatggttcggtcgtcgggttcggtcgttaggtgagcgttttggtttagtatttcggtcgttttgtcggggttgaggtttagttcggtcgtgaatttggttagcgggtagaggttttttagcgttattttggggtcgtgtaggaagtgcgtggtttgcgttagtagttcggtcgcggtcgttttgttttgttgttttaggtttagttgttcggtcgtgaggcgagttatagtaaagacgttttcggggaagtcgagtttgtttttgtcgtcggggtcggggacgtcggggagtttttttaagttcgttagcgtttcggtcgcgtcggtcgcgttttttacggcgtgtatttttatgtggttgatgaggttgcgttgttgtgcgaatttgtcgtcgtatatttggtattcgtagggtttttcgttcgagtggatgcgtatgtgtttcgtgaggcggtattggcgcgtgaatcgtatgtcgtacgcgtcgtacgcgaagggtttgaggtttaggtggttgcgtatgtggcgcgttatggttttacgttgcgtgaattttttttcgtagatggtgtatgggtagggtcgggttagttagtgcgttttttcgtgttgtcgtagcgtggtcgggtttttgtagtttttgtcgtacgacgcgtagcggtagggtcgtagtagtttttttaggttattcggagtttcggcgattttgttttcgtcgtttttaaaagggggttttaggtcggcggttttagcggttatttcggtcgtttcggttttgtcgtatagcgttttttttttttttacgtgagtttttacgtgcgcgtttagttgtttagagttggggaagtttttgtcgtacggaatgtatacgtataggttgttatcgaagttttcgggttcgttataggttaggtgcgggtatgggtagttttcgaggtggtcgttaggcgggttggggttttcgttgttatcggttttttcgttgttgtttttgtagtcgtcgtcgtcgtcgttcgcgtcgggttcgtttaggttgttagggtagcgcggcggcggcgttaggtcgagcgggggtttttcgggcgagacggtcgcgtttttattacgtttttcgtagcgttcgttgggggagtcgcgttttcggtttagttcgtcgttatagttatttaggttcggttcgtgttttatttagcgatagaggagattaggttcgtcggggcggtcggggggttcgggtttcgggttgtcgttgtcgtcgcgaaatgggtcggaaggcggtgcggttttttttagtttttggaagggtagcggcggtagcgacggtagggcgagaggcggttttttgtaggcggcggggtcggcgttgggagggttgttcgggcgcgggggtagttcgcgtttagttagcggtcgttttggcgtcgcggagttcggcgggttttttttggataggtttaggttataaagaggggagtagcggcgtttcgaggtatagagtgcggcggtcgggtcgggtttcgacgcgtatagttcggcgtagtgcgtgttgatcgcggtttttgggttcgagggcggtttcgcggtaggcggcggcggaggttcgattggggacgggtagtaggtttggatgatcggcgtggcggttcgtaggtttcggttcggtcgattatagggcgcgtagtcgtcgtcgtcgtcgtcgtcgtttcgtaggtggtagtatttgtcgtggcgtttgaggcgttttttgtatagcgttacgaggtcggggatttgtaggtagttggcggcggttagtacggcgtttaggttcggtttagttttcggggttacggtcgcggtcgtagtcgtttttgcgtcgttagttaggcggtcggtgtagatgaagtttagtattaggcggaatacggtcgggtttattatgttatggtttaggttgagtaggttgttatgtattattagggatttgaggtaggcgttgttggtcgttagtacgtttttgtgcgcgcggaagagggcgttttgtattacgatgattacgtcgtataagaagtttttggtgcgttggttgttgagttgtagtagtagttgtttggagtggtcgggcgtttttatcgtgtttagtatcgtttgtttagtatatttttttgcggggatatatatcggtcgggtgagagtcgtgcggcgttttggtcgtttggttttagttcggtattttttttttttattttttttttttttagttgagcgggggtattagttttgcggtttgggtatcggcgaaggatcggttgttttttggagtgggagtttaggtcggttcgttcggattaggagaaggagtaggaggtgagcggtcgtcggtggaggggaggttagggcggtttgtacgttttagggtatttggttgggtgttggggttttcgagaagaaaattgtttaggcgtagtgatttttttggagatagttattcgatttaagtaaaatgttcgttttaggaaaagttatttagggcggagaattttatttaagtagggagaaagggagtcgaggaattagcgtttttcgtttcgggagaagttgttttagttgggggaagtgatacggaggaggggagcgcggtgttcgttttggcgtcgttttggtcgggggttgttaattttcggtcggggttcgggcggcggtcgcgcggggagcggaggtagcggttgtcgtggcgggtagagcgcgaaggtcgggttcggcgcggggagggcgttatatcggggtaggaggttgaggtaggaagtaggtgggggggaggggggagttacgtagtttttaggggagggagggggtagcgtttcgggcgggtacggcgtatagtcggttgcggttttgattcgggtttgcgttttattcgcgtttcggtttcggtttgggttttatacgcgcgggttcggcgttttttttcgcggttttttcggttttttttttttcggaatttcgtcgttttaaatttggggaaaagttttttaattgtagatagggcgggaggagtgcgtcggttttaggttttcggttcgtagttttttttcgcggtttttaaattcggcggtagagttcggagtcgagttttgagtttttttgttcgttgttcgttcgttcgatttcgttttttttttggttcgcggggtttcgcggttcgttatttgcggtttcggcgggtcgggttttttttttcgcggcggtggtagtttttagtcgatgttttattcgtcgttgttaggtttcgagttgtgttagggtagcgtttttgttagtttcgt

SEQ ID NO: 8 - DNA, 2501 bp, Artificial Sequence
  chemically treated genomic DNA (Homo sapiens)
tttttatagtgtaaatgtgtttttattattttttggagtaattttatttaaaatcgtttttagtataaaatttaaatatttaaatatgattttgttggttttgtttttgtggttttattttttttttttttaaatttagttagtgtttgtgttgtttgtaatgtttttttttttttgtaggggtcgttattttaggttttggtttttttttagaaagttttttttttttttttttagcggggatagggtttgtttattttgatattattagtttatttatatatattggttataagtttaggttgtatcgttattgaaagtttattatttgattttgagtagtttgaggattttattaaaatttaggagatgtttagtaaatgttgattgaattatgattgtttttaatatataaacgtaagattatttaggaatatttgttaaaatgtttttgttttttgagattttattttgggaggtaagtagtgggggtttaggattttgtattttgatagttttttgatgtttgtatgtagaagtgtagggattattatattgataaatttttattatttttaagggggatttttttttttaggggttatttttggaagttttttaaggataggggtcgtatgttgtttttttaggttagtaattaaatttagaaaacgtttattgagtgaatgatgaaacgataggtgaatagatgaacgtaaggtgtcgagttaattatttttttatataagttttagtagtttttattgtttttagtcgtagaaatggtttttggaaggtaagttttttagcgagtggagttatttttaattatattttttaggattttaagggagtcgcgcgttttgcgtttatttttttattagaaatcggtaagttattgattttcgtttcgttttcgttatttttcgtttttttttgtttcgtagtcggcgtttagcggttttgtttgttcgtgtgtgtgtcgttgtaggttttatttatgggtttatcgttgaggttcgacgggcgggtggtattggttatcggcgcgggggtaggtgagtatgcgaaggttggaggtcgcgttttttgttgaggcgtagttggttgtttttttcgggtcggtatacgcgcgtagtcgtagttgaggttatttcgttgaggtggtggggaggggaatggttatttttgaggtatcgtattttttgaggaggaaagagtcggaaatatttggttttttaagtaggtatagttcgtttttttttagtatttcggtgtgggttttttaaggttttgtttgagaggagaggttaggttgggttgttgattgtaaaattgggtgaaagttttttttgatttttatttgtgggtatcgattgttattttttttgtaattaatttttttagatttttgtttagttttttaaaggattgaaaagtcgcgaggggcgggggttggaattcgttttttgaagcgtagagatgttagtttttgaaaagttattcggtcgtttagtgtttgtttttttttgtcgtaagattttaagttcgtgagaggattttttttaaagagggcgtttgataagagttttttttcgttggagtttgtatgtttagtaagttataatttgttttcgaaatttattggagttttggtagaggttgtaagtttaaatgcgtataggggttaggcgtatgatggagaaagaaaatgggagtaggatgggtatatttgaggaattggagagtagagaatttcgaagtggatcggttagtgggaaagttgtttgtattttaggagcggtaaaatggaaaattgttatgtgaaatagttttattttttaaagtataaaaaattaaaataaattatttatattaatatagatgttgtgtagtgagattttatattagttttttattagtgggtgatttttgtaatttttaagtgtagggattttgatattatgtatttttgattttttattggtagtattttatatttggaaaggttttaatgtatgaattatttgagttatatattaaacgttataaattggaattttgttaattaatttttatgtatttttatatttgtattgataaagtggttttttatgttgttttttagaaaatgtttttagtgttgatgaatagttaagtattttatatttatagttgtttggttatttttgtatgggtatgtatttgggtgtagttatattttttaaatgtttttaggaaaatattttgtttatattttgtttttattgtaaataatgtattttataacgtttggtgttttaaattttttttgatagtttttggataatttttatgtaggaggtttagggattatattttaagacgtttttgttatcgttaaggagatttttttttttaggggttatatttgaaaattatttaaggatagggattgttttttttgatattattagtatatttatatatggtatgtagtatattttatattagtatttagt

SEQ ID NO: 9 - DNA, 2501 bp, Artificial Sequence
  chemically treated genomic DNA (Homo sapiens)
attgagtattggtgtaaaatgtattgtatattatgtgtaagtatgttaatggtgttaaaagaagtagtttttatttttgaatgatttttagatatagtttttgaaaaggaaagtttttttagcgatggtaaaaacgttttagaatgtaatttttggattttttgtatgaaaattatttaagagttgttaaaaaagatttaaaatattaagcgttgtaaaatatattatttataataaaagtaaagtgtaaataaaatgtttttttaaaaatatttagaaggtatgattatatttaaatatatgtttatgtagagataattagatagttatgggtataaaatatttggttatttattaatattgaaagtattttttgaaaggtagtataagaagttattttattaatatagatatgaaagtatataggaattaattgatagaattttagtttgtaacgtttaatatataatttaaataatttatgtattagggtttttttaggtataaggtattattagtggagaattaaaggtgtataatgttaagatttttgtatttggaggttatagaggttatttattggtgagaaattaatgtaaaattttattgtatagtatttatgttggtatgaatggtttgttttaattttttgtattttaaaaaatggggttattttatataataattttttattttgtcgtttttgaaatataggtaatttttttattggtcggtttatttcggaattttttgttttttagttttttagatgtgtttattttatttttattttttttttttattatacgtttgatttttgtgcgtatttgagtttataatttttgttaagattttagtggatttcgagaatagattgtgatttgttaagtatataaattttaacggggaagggtttttattagacgttttttttaaagaaggtttttttacgaatttaaaattttacgatagagggaaataaatattgaacgatcgaatgattttttaggagttgatatttttgcgttttagggggcgaattttagttttcgtttttcgcggttttttagttttttaaaagattaggtaaagatttaagagagttaattgtaggaagagtaataatcgatgtttatagataagggttagggagaatttttatttagttttgtaattagtagtttagtttggtttttttttttaggtaggattttgggaagtttatatcggggtgttggggagaagcgggttgtatttgtttgagagattaggtgttttcggtttttttttttttaagagatgcggtgttttaagaataattatttttttttttattattttagcggggtgattttagttgcggttgcgcgcgtatgtcggttcgaaaagagtagttagttgcgttttagtaaggggcgcggtttttaattttcgtatgtttatttgttttcgcgtcggtgattagtattattcgttcgtcgaattttagcggtgagtttatgaataaggtttgtaacgatatatatacgaataagtagagtcgttggacgtcgattgcgggataggaggaggcggggaatggcgggggcgggacgagggttagtgatttgtcgatttttggtaggaagatgagcgtagagcgcgcggtttttttggaattttgggaaatgtagttaagagtgattttattcgttggaagatttgttttttaggggttatttttgcggttggaagtaatgggagttgttaggatttgtgtagaagaatagttaattcgatattttgcgtttatttatttatttgtcgttttattatttatttaataaacgttttttgggtttagttgttgatttagagaaatagtatgcggtttttatttttgaggggtttttagagatagtttttgggaaggaaagttttttttagggatggtaaagatttgttagtgtaataatttttgtatttttatatgtaaatattaggggattgttaaaatgtagagttttggatttttattgtttattttttaaaatagaattttaaggggtaaaaatattttgataagtgtttttaaatgattttgcgtttgtatgttgagaatagttatagtttaattaatatttattgagtattttttgagttttgataggatttttaagttatttagagttagatggtaaatttttaataacggtgtagtttagatttgtgattaatgtgtgtaagtgagttaatggtgttaaaataaatagattttattttcgttggggagaaagaggaaaaattttttgaaggaggattaggatttaaagtggcgatttttgtaaagaaagaagggtattataggtagtataaatattagttaggtttggggagaaagagggtaaagttataaaagtaaagttagtaagattatgtttagatgtttgaattttgtgttgaaaacggttttaagtaggattattttagagagtggtgggaatatatttatattatggaaa

SEQ ID NO: 10 - DNA, 2470 bp, Artificial Sequence
  chemically treated genomic DNA (Homo sapiens)
aaagatgattaaaagtttaattgtttatttgaagagttgatttttttatttttgtaataaagggtatttttagtagtttttgtttattttgtttattcggttttttttgtggttgtgtaaggttataatttttgtgttttagtaaatttgtgtatgtttatttttttttttgttattatttttttttttattttgttttattattttgatgtaaaattatttgttaattttatttgaaatgagaaattttaaggtttatattatttaaattttgttagatttttatttttgttatatggtttataatgtgttgggtatttttagatttgtttattaaaaagatgtaaaataaaataatgattatttttgtggattttttttttatttttgagatgttttttttggttgtattatttttttattttttgtttattgattagaggaggggttttaattatgggtgaattttatattttattgaagaggttatgttatatgtatatttttataatataatttatatttatatagtatttttatttttagtatatttttttttattaattttaataatattattgtaagttatgttgaagtagattgtaagtgtttatttataaattgtgaaatgaattaaaatgaaagggtaaagattaaattatgattaggtttgaaattaatatataagatttaattttttttaattaaagatttttgtaggtgatttttgtttgtaggatttttttttttttttagatgttattggattgtattaggtttattgtagattttagtcgttgtagaattaattagatttaagatgagttttttgattttttttggtagagttttttaattgttgaattttaatattgtcgtgattagttagtgttataatttgtttgttttattttgtgtaatggattttatattatagaggtatttttttaatgttaagatgtttaagtattgtttaagtgtaaattatttaatattttttagttattaagtaattaagataggtaggattttatttgttttaaaatgatttgatttaaattaaaaagagaatgtggattttttgaattttatttggttaattttaatataatttttagtattttataattttttttaaagtttttttatttggttattttttgtattttttttgttttttttttttttttttagttataataattgttagattttgttttatttttttttgatagtttttatttttaaggttatttattttttttaggtattttttggttttagtttgagtatagtagattttaagattatatatgttatagtataggttattatagttaattttttgaataaatgtgattgaattttatgttagtaatttttatttattatttttttattaaaaaggtttaaagtttttatttaatgtttttttttatgtttattttgttaaatgattgttttttaatgatattttagaattttagaattattttattatggaggatgtgtaagattagttttttattaaataaaaagtgtgaaatggaatatgtaattttattaatttattttggttttaaaattttgtgattattagataaaatttagaaataaaatagtattattaatataaataaatttttattataattatattttttaagttttgtttgtaagaatgggtaaaatatttttaaaattttgaagaaattattatttgatagaaagtttaatttatttgtgagaaggtaaatgtatttagatataattaaagtttttttttttattttaattttatttattttgaattaagattttattgttttatttttttagatgttgttatttgaataatattgttttgagattaaaaattagtatattaatataattttttttaaacgttttaagagttttgttttttttatttttttttttaaaaataagtagttattaaattttttagtagtgaattttaaaattttttttaattttataggtttaagggtagttaaggatggttgtagttttatatgattagttgttaaagtaagttgaggtattgaagatggagaatttaaattttcgataagagttagaagataattttaattattttataaaattggaaattgaggtatttaatatgaaggtattaagattgtgatttttaattgtagtttatttatttttatttagtatttttttttgtaaatttgaggtaagatattttatttaaaagtgtattttaaattaagtaataatatgtaaatttttttttgtaaaagttagtatttatatttttaaataagatatattgaatttatttagtgaattatataaagaaaataagtgtaaaattttaatggttagttagtttttagttttttttaagattaaagagaagagattaaatatagtattattgtattgaggtaaggttttttgtgtagtttatagaaattag

SEQ ID NO: 11 - DNA, 2470 bp, Artificial Sequence
  chemically treated genomic DNA (Homo sapiens)
ttagtttttatgaattatatagaaaattttgttttagtatagtgatgttatatttggttttttttttttaattttaaaaagaattaagaattaattagttattggagttttatatttattttttttatatgatttattgaatgaatttaatatattttatttaaaaatataaatgttaatttttgtaagaaagagtttatatattattgtttaatttaaaatatatttttaagtaaagtgttttattttaagtttataagagggaatattgaataaaaatggataaattataattaaaagttatagttttgatatttttatattagatgttttagtttttagttttgtaagatgattggaattattttttagtttttgtcgaagatttgagttttttatttttagtgttttaatttgttttaataattgattatatgaagttgtagttatttttggttatttttggatttataaggttaaaaaggattttgaaatttattattaaaaaatttagtggttgtttgtttttaaagaaaggggtaaaggaaataaaatttttaagacgtttaagaagaattgtgttaatatgttagtttttggttttaaaataatattgtttaagtagtagtatttaagaggatgaaatagtggagttttagtttaagataaatgaaattaaaatagaagagagaattttagttgtgtttgaatatatttgtttttttatagatggattaaattttttattaagtaataatttttttaaggttttaaagatattttatttatttttataggtaaaatttaggaaatataattatgataaaaatttatttatattagtaatattattttatttttgaattttatttgatagttatagaattttagagttagaatggattaatgagattatatattttattttatattttttatttgataaaaggttaattttatatattttttatggtgaaatagttttgaagttttaagatgttattaaaaggtaattatttaataaaatggatatgaaggagagtattaaatgaagattttaagtttttttgataggaagatggtaaataagaattattaatataaagtttaattatatttatttaaaaggttgattataatagtttatgttatggtatatgtggttttgggatttgttgtgtttaaattgaggttaaaagatatttaaagagaatggatgattttaggagtagagattgttaaagagaaatgaagtagagtttggtagttattatgattgggaaagaagaggagagataaagaagatataaaagatagttaggtaagaggattttaggaagaattatagaatgttaggagttatattaagattaattaagtaagatttaggagatttatattttttttttagtttaggttaaattattttggaataaataaaattttgtttattttaattatttaatagttaaaaagtattaagtagtttgtatttaagtaatatttaaatattttgatattaaaaaaatgtttttgtaatatgaaatttattatataaaataaggtagataggttgtaatattggttagttacgataatattggagtttagtaattggaagattttattaaaggaaattaggggatttattttagatttagttagttttataacggttagaatttatagtaaatttggtataatttaatgatatttgaggaggaaggggagttttgtaggtagggattatttataaaagtttttggttgaaaaaaattgagttttgtgtgttaattttaggtttggttatgatttaatttttgtttttttattttaatttattttataatttgtaaatgaatatttataatttgttttaatataatttatagtgatattattaggattaataaaaaaaggtatgttaaaaataaaagtattatgtaaatgtaagttatattatgaaaatatatatgtaatataatttttttagtaagatatagggtttatttatagttaagattttttttttgattaatgggtaaggggtgaagaagtaatgtagttaaaggagatattttaaaaataaaggaaaaatttataggagtgattattattttgttttatatttttttaataagtaggtttgaaaatatttagtatattataaattatatgatagaggtagggatttgatagaatttgaataatgtgaattttaaaattttttattttaaataaaattaataggtaattttatattaaaataataaaataaaataagagaaaaggtagtaatagagaaaaaaatgggtatgtataagtttattgagatatagaagttataattttatataattataaaaagagtcggatgggtaagatgagtagagattgttaaaagtattttttattataggaataaaaaaattaattttttagatgaataattaaatttttaattattttt

SEQ ID NO: 12 - DNA, 7001 bp, Artificial Sequence
  chemically treated genomic DNA (Homo sapiens)
aatgtaatggaaaaagagagattgtaaagttagaaggtttaggaattgttttttgattaggtgtggaaggtaagggaaaattagttttcgaagaagatagtgagattttaatttgggtggttggagagatagtgatgttgggtatagatacggggaagttgagaggaatattatgtttgagaatggtgatttatatttgaataagtttgtaatgtttagtagatcgttggaaaagtggggttggagatatatttaacggaggagttagattaatttttatttttttttatttgagagagttagtaagttacggttggaacgtgtgtgtttagtaggagagggtagggagggaagttaagagagttgggagttcgagtgaagtttttgttaaaggtagaagaggaaagtcggcgtagtatagtatatttttttatttatgtttattaagtttagggataaggtttattaagatgagtttggaagagaatgttggagagaaagtggttaagaaaattgtttttattgaattttttgggttaattttgattgtaagtttttgaataattaaagtttgtgaggagatagttaatttttttattttttttatgttaatagtgaataattgtagatttttttttttttttttttttttttttttgttttttttttttttttttttgaatatttttgtttttttttgggattggtttagagtatgggtggttattgttgatttataggaggtattattgttattaataaagggtaatagtttttttttttaatatttatttatatttagtatttatttttaatattgattatggagagagtttttttgtgtttaaatattgtaatattgggggttttttaaagtataaaaatatatatttgtatgatggtattattaatatttttatggttttttattttttttttgtattggttttaagagttatttataaattttttagtaattgtatagtgttttagggttagagatcggttatttttggtattgtgattagagttatttaatatttaaggtggtgattaatgtttggtaataaagtttttattgggtgttatgtgttttgggattttgagcgtgggtattttaggagtattttagtattgcgtgttagtattatggtcgagagaatagttgagaaagtggttaagaggtggatttatgtgaacgttattgggaaatgagagatttcgtttttaattacggttagtgtaattcgaaagtttaaaattagtttaaaataaaggtatttatttttattttatgtttatattttaggtttttaataatacgtattttttatatgtttatagaaagtagttaattgagttatttatggaaaggtttgtgggtttggttaacgaagtggaggagtattatattttagttggaaatatatttttagaatgttaaaatatttattttaaagtttggttttttggtgtaatcggaggtatggtaatgtttttgtttagagattgggggttagggttagtaaggtatttgatttatatgtattttagaaggtttttattgttaaattatattttttcggaaaaattatttatgttttattttgtaaatttgatatttatatatttttgattggtattttattttagtcgtaagattatgatttatagtaagtttgtttttttttttgtttggggtggtagtagaaagtatagggtattttttagtttttaagggtaggggtaaaggggttggggttttttttttttagtatagttttttttggttgtgttatattgttttttgtgagtagatagtaagtttttttttattttttattgttatttatttagcgttgtgtagtagtttagttgcgtgtttgtcgggaggggttgttaagtgttttgtttattggttgtttttcgaatttttgttattttacgtataaatatatttatatattttttttgtttagtttatatattgagttattcgtatatgcgagtatatttttttttttttttttattttttcggtttttgatttttataagtttatggaatatttttggaaagacgtttttgatttagtagggtaggtttgttttgatttttttttttgtagttttagtattttgagaaagtaatttatttttttggttagtgtttgtattttagtagggagatgaggattgttgttttttatgggggtatgtgtgtgtttttttttttttttaggatttgtaggattttttgtgttatttgtatataatttggtaggtttatattttttaagagttttatgaagtgttttttgtatgtgttttaaaaaggtatttgaaaattgaaagtgtgatttatggaaattaaattatttgtaaaaaattgttttggaaagtaatgattgttggttataaagggaaatatttgcgatgtatttaatgtgtttttaattttttatttgttgataatttatagttattaatgttaaattcgattttggttttagttatatttgtatattgtttaataatggtttatttttgtaagaattagataaaatgtatatttgatataaaatagttaaaaatgtaatttttagtaatagtaagtttggtatttagatagattatgaatatttcgttagatattttgttgggtgtttgggatagtaattaaaataaagtattgatagttgtattagagtttattaggttgtagtaaaggaagtttatttaaaagtataaattatttaagattatagacgtatgatatattttatttattttttgtttttttaatatgtatatatatatatatatatatatatatatatatatatgtgtgtgtgtatgtgcgtgtgtatgtttaatttttaatttagttaaaaatttttttttatttgttttttatttggatatttgattttgtatattttagtttaagtgaatcgagaagatcgagttgtaggattaaaggatagatatgtagaaatgtattttaaaaatttgttagttggattagatcgataatgtaatataattgttaaagttttggttcgtgatttgaggttatgtttggtatgaaaaggttatattttatatttagttttttgaagttttggttgtataattaatttgtggaaggtatgaatatttatgtgcgttttaattaaaggtttttttgaattattttttatatgagaatttttaatgggattaagtatagtattgtggtttaatataaatatataagttaggttgagagaattttagaaggttgtggaagggtttatttattttgggagtattttgtagaggaagaaattgaggttttggtaggttgtatttttttgatggtaaaatgtagttttttttatatgtatattttgaattttcgtttttttttttttagatgttttttgttagtttttttagttgttaaatatagttgtttgtggttggttgcgtatgtaatcgtatattttattttatttgttttatttcggttatagtgtagttttttttagggttattttatgtatatattacgtatttttagttaacgaggagggggaattaaatagaaagagagataaatagagatatatcggagtttggtacggggtatataaggtagtatattagagaaagtcggtttttggattcgtttttcgcgtttattttaagtttagttttttttgggttatttttagtagattttcgtgcgttttcgttttttggtcgtgaaatttagtttttatttagtagcgacgataagtaaagtaaagtttagggaagttgttttttgggatcgttttaaatcgagttgtgtttggagtgatgtttaagttaatgttagggtaaggtaatagtttttggtcgttttttagtatttttgtaatgtatatgagttcgggagattagtatttaaagttggaggttcgggagtttaggagttggcggagggcgttcgttttgggattgtatttgttttcgtcgggtcgttcggttttatcggattcgtaggttttcggggtagggtcggggttagagttcgcgtgtcggcgggatatgcgttgcgtcgtttttaatttcgggttgtgttttttttttaggtggttcgtcggtttttgagttttttgttttgcggggatacggtttgtattttgttcgcggttacggattatgattatgattttttatattaaagtatttgggatggttttattgtattagatttaagggaacgagttggagtttttgaatcgttcgtagtttaagatttttttggagcggtttttgggcgaggtgtatttggatagtagtaagttcgtcgtgtataattatttcgagggcgtcgtttacgagtttaacgtcgcggtcgtcgttaacgcgtaggtttacggttagatcggttttttttacggtttcgggtttgaggttgcggcgttcggttttaacggtttggggggtttttttttatttaatagcgtgttttcgagttcgttgatgttattgtattcgtcgtcgtagttgtcgttttttttgtagttttacggttagtaggtgttttattatttggagaacgagtttagcggttatacggtgcgcgaggtcggttcgtcggtattttataggtattcgcgttcgcgtcgttcgtcggggtggtcgtcgcgttcggtaggagggagggagggagggagggagaagggagagtttagggagttgcgggagtcgcgggacgcgcgattcgagggtgcgcgtagggagttcggggcgcgcggtttagttcgggggttttgcgtgtagttcgcgttgcgtttagagttaagttttttcgtcgggtagttgaaaaaaacgtattttttatttatttatcgttcgtgcgagaggtagattcgaaagttcgggttttttaataaaatatacgttggaaaattagataaagtagtagttatttgtgggggaaaatatttttaggtaaataaatacggggcgttttgagttatttgggaaggtttcgtttttggtatttaaagttgggggtgtttggagttagtagagtttagtagagttttatttatttttttaatgtttttgtttaatgtgttttttaaatttttttttatttagattatttgattggaaatatgttagttatgatgatgattttttgggaagcgatttttgttattcgttttttttttttttttattttacgttttggggttttagagagcgattgggagttgaatgggtttgatttcggagttagttggttgagttcgcgttggagcggattgttggtatgtgatttttgatagtcggaaatttgtaggtgtttcgcgagtttaaaataagttatatggaagtataagtgtttaaaaataattttttgttagtttagtgataagtttgttttattcggggagaatgtttcggagtggcgtgcgggttagttagggtttgcgtttcgtagttattgtggaaggagcgcggtcggtttaggatataggagattattttgtgattttaatggcgaaggttgtgtgtttttattttaatttttttttttataagaattgttttttttttttttttttttttttttatttttttttgtttagtttttttttttgttttttgttttttgtttttttgatgggtttgtagagggattaggtgggcgtttttggtgaatatttttttaggtggttataggataggtgtatttcggattgggtttggaagttttagggcgttatatggttgggttttgaattaggtattttttaattgtatattggtattcggattggtgtttttatatttttttgttttgtaagtcgtggattagtttttgtttagtattttgtttttagggatatttatagtagaaggaaggggattaaagtgtagtttggttttagaggatattgaagggtagattttgggggtatttagtgtgtatttttagtcgttttggagaaatttagagtattttatagttacgtagatttaagttgtttttatttaaaagataaataatgaataaaatttttaaaggttggtatattttaaattaattttatttgttttaatttagggttaaaatagagaaaaaggattttttttgtttatttttttttttttaaatggaagaataaagtatagcgattaagtttaattttatataatatttaaaattgtttgatgtgaaggaaggtattggtatgatgtgaattttataattttatgatggattttagaaattatttttttttttatttaatttttagtttttttattgtaaattaatgttgttgaattttaatgggtattaatgagattgttttttggtagattatttattgttttgttaataattataaagtgaatttggttaaatatagaggggatcgtattttatttaaaattgtttattattttagtgataagtggtattagtgtaatatgttttattttatattttttgtattatatgatatttaaatatttttagaataataaaaaaagagataaggaatttaaaaattaaaaaaaaaatttgtataaatgggattttgtgtggaaatttagttttagaatgattttttttgtgttttatttttcggattatttttttttttttgttagaattttgtttgttattatttagtaaggaaaagaagtatttatgtaagttttttatatggatagatattatttagtatttttttttttttagtttttttgtttaaatgattttgggtataaaggaaaggattgattgggtttttttaggaaattttaagttttttaagtagtttttaaaagttttggggttgaaagtagtgtttttaaattgtttgttatgatttagagggttatgaatttagtttagtgagtttagaatattttttaaaaggattaaaatggaaaggaatataatagaaaatattagagtgtatggtatttcgtaaggataagttttgt

SEQ ID NO: 13 - DNA, 7001 bp, Artificial Sequence
  chemically treated genomic DNA (Homo sapiens)
ataaaatttatttttacgaaatattatgtattttgatattttttattatatttttttttattttagtttttttaaaaaatattttagatttattaaattgagtttatgattttttgggttatgataagtagtttgaaaatattgtttttagttttaaaatttttgagaattatttaagaaatttaaagttttttaaaagagtttaattaatttttttttttatatttagagttatttaagtagaaaaattgagaggggaaaaatattaaataatatttgtttatatgaagaatttgtatagatgttttttttttttgttggataataataggtagaattttaataaaagaggaaagataattcgggaaataaaatataggaaaaattattttaaaattgaatttttatatagagttttatttgtgtaagttttttttttaatttttaagttttttgtttttttttttattattttaagagtgtttgaatattatgtaatgtagaaagtgtaagatagggtatattatattgatattatttattattgggatgatgaataattttgaataagatgcgattttttttgtatttgattaggtttattttgtaattattagtaaggtagtaaataatttattaaggagtagttttattagtgtttattgaaatttagtagtattaatttgtaataaaagaattgaaaattaaatagggaagaaaatggtttttggagtttattataaggttatggaatttatattatattagtgtttttttttatattaagtagttttaaatgttgtgtggaattagatttaatcgttgtattttgtttttttatttaaaaaaaaaaaggtgggtagaagaaattttttttttttgttttaattttaaattaaaataagtaaaattaatttgaaatatgttaatttttaaaagttttgtttattgtttgttttttgagtaaagatagtttggatttgcgtggttgtgggatgttttaaatttttttaaggcggttgaagatgtatattgaatatttttagaatttgttttttagtattttttggggttaaattgtattttagtttttttttttttgttataaatatttttggaaatagaatattgaataaaaattggtttacggtttataaggtagaaagatatagggatattagttcggatattagtgtatagttgggaaatgtttaatttaggatttagttatgtggcgttttgaagtttttaaatttagttcggggtatatttgttttgtggttatttaggaaggtgtttattagaagcgtttatttaatttttttgtaggtttattaggaaaataaaaaataaaaaataaaaggagaaattgggtaagagaaaatgggagggagaggagagggagaaagaataatttttgtagggaaaaaaattaaaatgaggatatataattttcgttattgaagttataaagtggttttttgtgttttggatcggtcgcgttttttttatagtggttgcgaggcgtagattttggttgattcgtacgttatttcggggtatttttttcgggtgggataggtttgttattgggttggtaggagattatttttaagtatttgtgtttttatatggtttgttttaaattcgcgggatatttataaattttcggttgttagaagttatatgttagtaattcgttttagcgcggatttagttagttaatttcgaaattagatttatttaatttttaatcgttttttaaagttttaggacgtggggtggggaggaggggaaagcgggtgataggaatcgttttttagaaagttattattatagttgatatatttttaattaaatagtttagatgaaaggaaatttggggagtatattaaataaaaatattaaaaggataaataaaattttgttgagttttgttaattttaaatatttttaattttaaatgttaagagcgagatttttttaagtgatttaaagcgtttcgtgtttatttgtttggaggtgtttttttttataaataattgttgttttgtttggttttttaacgtgtgttttgttaggaagttcgggttttcgggtttgtttttcgtacggacggtaagtgggtggagagtacgttttttttagttgttcggcgagagaatttgattttgaacgtagcgcgggttgtacgtagaattttcgggttgggtcgcgcgtttcgggttttttgcgcgtattttcgggtcgcgcgtttcgcggttttcgtagttttttaggttttttttttttttttttttttttttttttttttgtcgggcgcggcggttatttcgacgggcggcgcgggcgcgggtatttgtagaatgtcggcgggtcggtttcgcgtatcgtgtagtcgttgggttcgttttttaggtagtagggtatttgttggtcgtggggttgtaggaaaggcgatagttgcggcggcgggtgtagtagtattagcgggttcggagatacgttgttgagtggggggaaattttttaggtcgttggagtcgaacgtcgtagttttagattcggggtcgtaggggaggtcggtttgatcgtagatttgcgcgttggcggcggtcgcggcgttgaattcgtaggcggcgttttcggggtagttgtatacggcgggtttgttgttgtttaggtatatttcgtttaggggtcgttttagggggattttgagttgcggacggtttaggggttttagttcgtttttttggatttgatgtagtagggttattttagatgttttggtgtggagggttatggttatggttcgtggtcgcgggtagggtgtagatcgtgttttcgtagggtagaaggtttagaaatcggcgggttatttggaaaaagagtatagttcgaggttagaggcgacgtagcgtatgtttcgtcgatacgcgagttttggtttcggttttgtttcgggagtttgcgggttcggtgaagtcgggcgattcgacgggagtaagtgtagttttaggacgaacgtttttcgttagtttttgggttttcgggtttttaattttaagtattggtttttcgagtttatatgtattataaaggtgttggaggacggttagggattgttgttttgttttgatattggtttaaatattattttaggtataattcgatttggagcgattttaaagagtagtttttttgaattttattttatttgtcgtcgttgttggatagaggttgagttttacggttagggggcgggggcgtacgaggatttgttaaaggtggtttagggaagattgggtttaaaataaacgcgaaagacggatttaggggtcggttttttttaatgtgttgttttatgtgtttcgtgttagatttcgatatatttttgtttgtttttttttttgtttgatttttttttttcgttggttagaaatacgtagtgtgtatataggatgattttggggaggattatattgtaatcgagatagggtagatagaatggggtgtgcggttgtatacgtagttagttatagatagttatatttagtagttgggggaattgatagggggtatttgaggggaagggggcggagatttagggtatatatataggaagagttgtattttgttattaggagaatgtaatttgttaggattttagtttttttttttgtaaaatgtttttaaagtagatagatttttttataattttttgagatttttttagtttgatttgtgtgtttatgttggattatagtattgtatttggttttattaggaatttttatgtgaaggatgatttagaaaaatttttggttagggcgtatatgggtgtttatgttttttataggttggttatgtaattaaaattttagaaaattgaatataaaatgtgatttttttatattaaatataattttaggttacgaattaaagttttggtaattatgttatattgtcggtttggtttagttaatagatttttaaaatgtatttttgtatgtttattttttagttttataattcgattttttcggtttatttgggttaggatatgtagaattaaatatttagatgaaaaataaatagaaaaaagtttttaattgaattaaaagttaaatatgtatacgtatatatatatatatatatatgtgtatatatatatatatatatatatatatatatattaaggagataaaaaataggtgaagtatattatgcgtttataattttggatagtttatatttttgaataaattttttttgttgtagtttaatagattttgatataattattaatattttgttttaattgttattttaaatatttaatagagtatttgacgaagtgtttatggtttatttaaatgttaagtttattgttattaagagttatatttttgattattttatattaagtatatattttatttaatttttataaaaatagattattgttggataatatgtaaatgtagttgaagttaaaatcgagtttagtattaatgattatagattgttagtaaataaagggttaaaaatatattaggtgtatcgtagatatttttttttatggttagtaattattattttttaaagtaattttttatagatgatttaatttttataaattatatttttaatttttaaatgtttttttaaaatatatgtaaaaagtattttatagggtttttaaaaaatgtgaatttgttaaattatatgtaaatggtataaagaattttataagttttgaaagaaaaaggagatatatatatatttttatggagaatagtaatttttatttttttgttaggatatagatattagttagaaaggtaagttgtttttttaaaatgttaaagttatagagagagaaattaaaataagtttattttgttggattaagaacgtttttttagaaatgttttatgggtttgtagaagttaagggtcgagagagtgagaaggaaggaaggaatgtgttcgtatgtgcgagtggtttagtgtgtgaattaggtagagagagtgtgtggatgtgtttgtgcgtggaatggtagggattcgggaagtagttagtaggtagggtatttggtagtttttttcggtagatacgtagttgggttattgtatagcgttggatgaatggtagtggggagtgaggggagatttgttgtttgtttatagggagtagtgtggtatagttagagaaagttgtattggggaggagaaattttagtttttttgtttttatttttggaggttggaaagtattttatgttttttgttgttattttaagtaagaggaaaaataggtttgttgtgaattatagttttacggttaaaatagaatgttagttaaaagtgtatggatattaagtttataaaataggatatgggtggttttttcgaaagaatataatttaataataaaagttttttgggatatatgtggattaaatgttttattggttttagtttttagtttttgaatagaggtattgttatgttttcgattgtattaggaaattagattttggaataaatgttttggtattttagggatgtgtttttagttgaaatgtaatatttttttatttcgttaattaaatttataaatttttttatgaatagtttagttgattgttttttgtaaatatgtgaaaaatacgtattattaaaagtttaggatatgaatataagataaaggtagatatttttgttttaaattgattttaggttttcgagttgtattgatcgtgattgggaacgaggttttttattttttagtggcgtttatatggatttattttttgattatttttttaattattttttcggttatagtattaatacgtaatattgaggtgtttttagagtgtttacgtttagggttttaggatatatgatatttaatggaggttttgttgttagatattagttattattttggatattaaatgattttaattataatgttaggagtggtcggtttttggttttgggatattatgtagttattgagagatttatgagtggtttttgagattagtataaaaaagaaatagaaagttataaaaatgttaatgatgttattatgtaaatatatgtttttgtgttttgaaagatttttagtattgtagtgtttgagtataggagagttttttttatagttagtattgaaaataaatattggatataaataaatattgaaaagaaagattgttattttttgttggtgatagtggtgttttttgtaggttaataatggttatttatgttttagattagttttagaaaaaagtaagagtatttagggagggaggagagaggaataggggaaaggagaaggaaaggaaaggggatttgtaattgtttattattgatataggaagaataagaaggttagttgtttttttataggttttgattgtttagagatttataattaaagttagtttaagaagtttagtaaaggtagtttttttaattattttttttttagtatttttttttaaatttattttggtgagttttgtttttgggtttggtgagtatgggtgggaaagtatattgtgttacgtcgattttttttttttgtttttggtaaaaattttattcgggtttttagtttttttggtttttttttttatttttttttgttggatatatacgttttagtcgtgatttattggtttttttaggtgaagaagggtaaagattgatttggttttttcgttgaatgtgtttttagttttatttttttagcggtttgttgggtattgtaggtttgtttaaatatgagttattatttttaaatatggtgttttttttaattttttcgtgtttgtgtttagtattattgtttttttagttatttagattaaaattttattgtttttttcgagggttgatttttttttgttttttatatttaattaagaggtaatttttaagttttttagttttataatttttttttttttattgtatt

SEQ ID NO: 14 - DNA, 11001 bp, Artificial Sequence
  chemically treated genomic DNA (Homo sapiens)
ttaagttagatgttttttaattatttgtggataggttaggtatattttgagtttaattttattttataggtttaatattttggagttagaaagtttttaggtaaaaagtttgaagggggtttttttatgttattagatggatttttgtatttttagaagattttttatattaggaaagattaaagtattaaggtaattttttttggttttttgggataattttaggttttggtatgagtggtttggaagtttttgttttagttataatgtttatatatttttggaattgttttgtagggtttgttttttagtataattttttttttaagttttattgtagttatagtttattagttttgtttagtgataattaagaaattaagaattatgtatttacgtttatttttttagagttatttttttttaggataaagtttagggttttgttattgggttttgttaggagtcgtagtcgtaggggttgtttattatttaatagttttcgtaagtatattgtgaaggggaagtaatgattagagatagggttagttgtttagtttttgtatgtttaggtgtatgcgtatatatttttatatagggtagggtggggtgggaagtttattttggtcgtgacgtttagcgcgtttaaagagtgtaaatttgcgggggttatttattattaagaattttcgtagtagggttttaatgattttacgtgtttttgtgcgtgatagtatatgtaggtgtttgatagtatttttgggtaggtaaaaggaagtgcggcgtttgatatttgtgtttcgttttgggtttcgtcgggtattttgtaaggagggtggttttttgttggagggtatagagatagggcgtattagttttagttgaattttgatgaagtttgtgtaagaatcgtttttgttttaaagaaatagagaaattaaattttgataataggttttaggtgagatgttagtttatttggggttaggttgggtatgtataaattattgtttgcgtttatttaagataattttagttgtgattttttgagtattaggtatatagttgggtatttgtttttttttacgttttttttttgagtagttaatttattaagtttatgaagaggttgttgttgatttgggtattgtattttttgattttttgtttaattttagtttgagaaaggttaggtgttttttattttataggttcgttttgtaagatgggttagtatggatatagggtttttgaggaatttagggtttttttgaaaaatggtttttggggtagtttttggaaattgattgtttttggttttttgtttttgatgtatatatatatagttggtgtttattttgaatttattattgtttttggttttgtatgttttgggtggataagggaaagatagaattatttggtttttttttgttgtttgtttagggttttagtattgaatgtagttttaaggatattatagaagtaggggtaattgaaggtatatggttaggggttaggaatagttgagggattttgaagagggatttttatttaaagtaaaattaggttgggtgtggtggtttatatttgtaattttagtattttgggaggttaaggtaggaggattatttgatttttaggagtttgagattagtttgggtaatatagtaagattttatttttattaaaaaaagaaaaaaaaaaattagttaggtgtggtggtgtgtttgtagttttaattgtttaggaggttgaggtgggaggatcgtttgagttcgggagattgtagttatagtaagttattatcgtgttattgtattttagtttggggaattgagtgagattttgttttaaaatataaaaaataaaaataggttgggtacgttggtttacgtttgtaattttagtattttgggaggtcgaggcgggtggattatttgaggttaggagtttgagattagtttgattaatatggagaaatttcgtttttattaaaaatataaaattagttaggcgtggtggtatatgtttgtaattttagttatttaggaggttgaggtaggagaattgtttaaattcgggaggtggaggttgtagtgaattgagatcgtgttatcgtattttagtttgggtaataagagcgaaattcggttttaaaaaaaaaaaaaattagtaaaattatattttaattgtatattttgattatagtattttagttgagttggagtgagggtttgttttggagaaggtagtttattttttttttttgtttcggtacggggttatgatttattgtagggtgagaggagtggagagtggtgtatattagtagtttagttattagtggatagagtagtatttggagttagttttttatgttttatatatagtgagaaaaattattgtgatatgatgtttaattttgatttaagttgtataaaaggtagttttaggttaggttttaatttgttagaggtatataggtagttttttggtgggtttttgtatttgtttgtgttgtttggagatttggtttaaagattttttttttttttgagacgaagttttattttgtcgtttaggttgtagtgtagtggttggattttggtttattgtaagttttgttttttgggtttaagcgatttttttgttttagattttcgagtagttgggattataggcgcgtgttataataatattcggttaatttttgtatttttagtagagatgggattttattatattggttaggttggttttgaatttttgattttaagtgattcgtttgtttttattttttaaagtgttgggattataggcgtgaattatcgtattcgatttagagatttttaattcgattatttattttttattttatttagggattggatttttgtcggaagggtggagtgtgggatagggtagttagggttttgaatcgattttttttttttagatttttttggttttattgtattagttttattttttgttgacgttagataggttttagttagaatgcgagtgttatagatatagttaagtttagcgttgattaatattttgttttagaagaatttttataaggttttttgtagaatgattttgtgtttagtttaggagagttagggttttttttgatttcgttttggagtttttttaagtatttaaattatttgatggggataaatggagaggatagatgagggagtagggtggagcgttttagtagaatgttttttatttagaattcgttgttattttgtagttagtaaggatgtggggttaagaattaaggttagggttttataggaaaaaggtaaagggggaggggtgggaatttaagtttatttttttttttaagtatttaaaggttttttggatggagaagagtattggagtaaaaattttagtataaattttattggggatagtgggtaattttgtcgggttagtaaaaataaatggtgtgggttttggaaaatgagggttggaggttgtgaataaagtagtggatgtgtttgtttagtatattaacgggaagaagtatttagatgggaggagtattaggggtaggagaaatgttagatagattttagtgttagggtaagaaggaagattattttgtttgtagaatagggagggtatagggatggtgttaatttgtttttgtgatggttttgagtttttatttaataatgagaaagtttgtttttttttttttttttggatgatttaggagttttgggttgggatgtagtgattttatttttagtttttttttttttggtgatgaatttttttatttttatttagaaaatagatttggattagaggtattgtatagtttttttaggattttaaaggaggaagagtttttttttttgtttttaaagttgtttgttggaagaggattttaatagttattttagtcggatgtatagtaggattatggaattttttttttgtattatagggatttattttttattttattattgtttataaaaattgatggttttttttttgagatagagtttcgttttgttttttaggttggagtgtagtggtgcgattttggtttattgtaatttttgttttttgggtttaagtaattttttgttttagttttttaagtagttgggattataggtgtttgttattataattggttaattttttgtatttttagtcgagacggggttttattattttggttaggttggttttgaatttttgattttatgatttatttatttcggttttttaaagtgttgggattaaaggtgtgagttattgtatttggtttaaaattgatgtttttttttttttttttaatatataatttgggattttttagttttttattttttttttttttttttttttttttttgagatagagttttgtttttttatttaggttggaatgtagtggtttagtttcgatttattgtaatttttgttttttgggtttaagtgatatttttgttttagtttttttagtagttgggattataggtatatattattatggttagataatttttttgtatttttagtatagacggggttttgttatgttggtttggtaggtttcgaatttttggttttaagtgatttgtttgttttggttttttaaaatgttgagattataggtatgagttattaagtttagtttttttttttttttttgagatagagttttattttgttatttaggttggagtgtagtggtacgattttggtttattgtaatttttgtttttcggttgaagtgatttagttttttaagtagttgggattatagttatatattattatgttcggttaatttttgtatgtttagtagagatagggttttattatgttggttaggttgatttcgaatttttgattgtaaatgatttatttgttttggttttttaaagtattggtattagaggtgtgagttatcgtatttggtttttttttttatttttgagatagagttttattttgttatttaggttggagtgtagtggtacgattttggtttattgtaatttttgttttttaggtttaagtgatttttttgttttatttttttaagtagttgggattataggtgtgtattttcgtggttagtttttttttttaattggttagtgttttgtggtttttttatttttttatagtggaaaatggtttaggattgattgatatgaagataagtttaggggtttatatttaatttaatttttgtatttaagttttgggttaagattttggcgtgttgagtattatttattttgtaaggaattttgtaaaattttatttgaagtattatttataattttattttttttatttaaataaggattttcgttttatttttgttaggtatattgagttttatagtttttgttttttttttttggtgtttaggtttggttttttgagtttggtggttatattaatggtatttggtatatagtttttcgataatggggatatttaggaggtttcgagatattttatagttttgggttagtaatttggatttttttttttatttttttaggtattttataatttagttttttttttttttgtgggtaaagtgtttttgaatgtttatggtttaaaataagattttttttttatttatttttaaatttttttttagatttattttagaggaagggaatagaattttttatattttagtagttggtgataggttagaatagggaagaggtgagggtttagttggttttatataggagtgtagatggaggagtaggatttttttttgttttttaagtttttttaaatatattttttaatttttggcgaggatttttttttttttatatttttttttagtttttttaaggagggagtaggagtattcgaacgcggaaatcgaggtgttagtttaaattgttcggtcggttttagttatagttggataatgttcggtttaggtttattataagttatatagttgtttttttcgtgtttaatttgtttgtgatagaaattaagggggtttcggtatttagtatttaggcggtggaatcggggttttacgtacggtttcgcgggtaggttttcggttaggattcgcggggagttacgtagttaggagggtggggttgtttatcgatttaggacgcggtaacggatcggggagggcggagttttagcgatcgttttttttttcgttcgtcggtattttttggtttttatttggtttcggcgcggtttgcgagttagcgaggttcgcgcggtgaagtattgttcgagtttcgagttcgagtttttttggttgtagtagttattgttcgtcgtgttgttttaggttatttcgaaagaaggcgttttcgtttcgtttatagtcgtattcgttcgttttttagttttgcgcgttcgtagtcgttaattatcgtttcggtcgcgtgcgtgcgtgtacgcgtgttagtgtgcgcgtgcgttcgggttagagtcgcgtcgtaatcgttaagattgaaacgtagatcgtcgggatttagtttttgttttattggggtaggaacgtcggggcggggatacgtacgtttcgtttttaggaatgattttatcgtttcggagttttatttatagattttatttattatagggaacgggggcgggtgttagcgttcgggtaagcgtataagagtggtttttggtcggaggcgagggcgggaaggtgcgggaagtgcgcgtgcgcggagtttgggttagtttgggttcgggttcgtttgtagcgggtggagtatttgcggagtcggtaatttaggttttttttttagttttcgcgtagaattagtttttttgtgtcgtcgggaaatcggtaattagaacgttttttgcgcgcggtatttaggtagttttcgagaatgtttgtattgtggtttgtttattttcgttttttttatacgttttcggtttcgcgtttattacgttcgtgtttcgttttatcgcgggtttttagtttaggtttcggggttcgtaatagtttaggtagacgagcgcgcggtagcggtagtggtaggtgaatttgtaatttgtagagaggtttggcggtgaggcggaggagttttaggtcggggaaatgtttcggagattgaagggaagttttagggagagggtcgttgttcgttaggtttcgtaggttcgatttattttagtgggttattttatattgttacgcggattttaatgttggttatttgggcgtttggaaatcggtcggaaggttataggtagagaggtttgtttaatagttggatttttatcgtttagtatagaattttttttttttttattggtaattaaaaaaataataataaaaaattgcgttttgtttttgttatttaggttggagtgtaatggcgcgatttcggtttatcgtaattttcgttttttgggtttaagcgatttttttgttttagttttttgagtatttaggattataggcgttcgttattatgtttagttaatttttgtatttttagtagagacggggttttattatgttagttaggttggttttaaatttttgattttaggtgatttattcgtttcggttttttaaagtgttgggattataggtttgagttatcgtattcggttttttattgggaacgtatatggaatatatttgtttatttatttgaaggaaaaattaaatatttttaatttacgtttgttttgtggttgttatttgtttttattttttttagattaagatattggtttttatatattttaatttttcgtttttatttttttttttatttattttagttaggtttgtttttttttttaggaaattgttcgggatagggtttttagcgatttgtgtattattaaatggaatttagtgttttattttttatttttatttttttagtattatttgaagtttgttttttttgatttttaggggttatatttttttagttttttttttattttttgtagtttttgtttagtttttttgtagattttgatttaatttttatattttacgatgaagtttgggtttagttttgattattggtttggtttgtttatatttatttgttttagatttacggttgaaatattgatttaaatttttagattagatttttcgtgttagtatttttattaggatgtttaaaagacgttttaagtgaatatggttaaaatttaatttttttttttttagttttattgttatatttgtttagttttttttttgtagtaaaaatggttattaggtttttagttattggagataaaagtttaaatttatttttgattttttttttgtttttatttttgataaatatgtttaaattattttgttttttatttttatggttattttatttttttttgagaacgttgtaatgttttagttttgttttttttttttttttttttttttttgagatagagttttattttgtcgttaaggttggagggtagtggtacgatttcggtttattgtaattttcgttttttgtgtttaagtaatttttttattttagtttttcgagtagttgggattataggtattcgttattacgtttggtttatttttttttattttttagtagatatgaggttttattatgttggttaggttggttttgaatttttgattttaggtgatttattcgttttcgtttttcgaagtgttgggattataggtatgagttatcgcgttcggttttttgtttattttttgtattttgttataattttgtgttttttttagttgaatttgtgatgtttttttgtatcggatgagagggtttttatgtatatatagatttgggatattatttatttataagtttttaaataggttagagtagtgatgtttaatttagattttatgttataataatttggggagtttttaaaatttattgatgtttagggtttatttttagtagttgatttaataggtttgcggtgggatttaggttagcggggaggattgtaaaagtatttttggtgattttagttggtgtttatttaggggagagtaatttttgtttgttggcgatttttaggggtgtagaaggattgttgggtgtgtggttgcgtgtatattttagtatttgatttattgggttagaaaagggtgtttgttaaataaagatttaataaaatttttgtttgtagggggtttattaaaggttttaaatttttttaggtttttttttataggtggtaatttttttttattttaaaggttttggagggggttatgagtgtttgagaagaggtaagtttgggaagatggatttcgaggatagtaggtataaatttttttttaagaagggttaaggtattttaaagataagaaatttaaaattagcgtatttttatatataagtagttatttttgtttatttgtggtttagatacgagtggagtgcgataagggataaattattttcgcgtattttttagcgatggggcgaaagtaacggatttagttttcgggagttgttttcgtcgattttttttgtcgcgatttgattcgcggcgattgcgttgttttttggttgtttttttcgttttcgtaggcgcgcggggttattatttacgcgcgtattgtaggtttttgcgtacgacgttttagatgaagtcgttatagaggtcgtattacgtgtgcgtggcgggtttcgcgggttggaagcggtggttacggttagggattagttgtcgtgtggggttgtacgcggtgtttcgcgcgatgcgtagcgcgttggtacgttttagtcgggtgcggttttttttagcgcgtttagcgggtgttagttttcgtagtttaatgagtttaggttttttcgatatggttcggttgggttcgtgtttcgttggttttgggcgttagtaagcgcgggtcgggcggggttatagggcgggtttcgattttagcgttttttttaggatttagattgggcggcgggaaggagttgaggagagtcgcgtaatggaaatttgggtgtagggattgtggggttcgaaggcggggttgggcgcgttttcgtagagtttttttcgttttgtttttttttttttttttcgttttttttttatattttatttcggacggttataacgacggcgatcgtaaagtattacgcggagatattcgtgtttttggaggttagttttattgtgttagaggaagagggtttttatattcggttttggtttttttggttcggtttgttgaagtaatatatttggtttatttattgggtggggtaggaagtttcgagtttttatttggggtgaggaggagggagatcggttagtagttttatcgttcgttttgttttttattgcggagattggggtttcggtagaggttggatcgtgattttgaggtttaggggtgtattttgggtggatttttttggtatgggtggtcggtttttagtaattgtagtttttatttggttttgttattttgggttgttaggatataagtttttttatgttttttttagtgtttgatttggtattttttgtaggtaggtgggtattgaggatggtaatgtatgtgggggatgtgggagtagggtttagaggtttaaggttttaggatatttttatttgtagtaatattatttattttggtatcgtgagtagcgtttagaagtttttgtattgtagtaagtatagcggggtcgttttggagttattgtttttagtatatttagtttgtaggttttagtttatttgggggaaagttaggaaggtttgattggttttggaaggtgggggtattttatttatatttatgttttttgtattttttttattttttttgttatttttataggttttattttcgcgtttgtagtcgtaggttttgttttgaggggttgaatatatgttggagttggtgtttggtaattgtttgttatttgtttttgttttttcgttttagtcgtttttagatttttgggatttagga

SEQ ID NO: 15 - DNA, 11001 bp, Artificial Sequence
  chemically treated genomic DNA (Homo sapiens)
ttttagattttagaaatttgggagcggttggagcgagaaaatagaggtaagtggtaggtaattgttaagtattagttttagtatgtgtttagttttttagagtaggatttgcggttgtaggcgcgaaggtaaggtttgtggaaatggtagggagggtggaggggatgtaggaggtatggatgtgggtggggtgtttttattttttagggttagttagatttttttgatttttttttaggtgggttgagatttataggttggatgtgttagaggtagtggttttagagcggtttcgttgtgtttattgtagtgtagaggtttttaagcgttgtttacgatgttagaatgagtggtattgttgtaggtgagggtattttagaattttggatttttaagttttatttttatattttttatatgtattgttatttttaatatttatttgtttgtagggagtgttaagttaagtattgggaaaagtatggaaagatttgtgttttggtagtttagggtgatagagttaaatgagggttgtagttgttgagggtcgattatttatgttaagggaatttatttagaatgtatttttgaattttaagattacggtttagtttttgtcggagttttagttttcgtagtggagagtagagcgggcggtaaagttgttgatcgattttttttttttttattttaagtgaaggttcgagattttttgttttatttagtgggtaggttaagtgtgttgttttagtaaatcggattaggagggttagggtcggatgtggggattttttttttttagtatagtaaagttggtttttagaaatacgggtattttcgcgtggtgttttgcggtcgtcgtcgttgtggtcgttcggggtggggtgtgaggaggggacgaaggagggaaggaagggtaaggcggggggggttttgcgagagcgcgtttagtttcgttttcgggttttatagtttttgtatttaggtttttattgcgcggttttttttagttttttttcgtcgtttagtttggattttgggggaggcgttgaagtcggggttcgttttgtggtttcgttcggttcgcgtttgttagcgtttaaagttagcgaagtacgggtttaatcgggttatgtcgggggagtttgagtttattgagttgcgggagttggtattcgttgggcgcgttgggaagggtcgtattcggttggagcgtgttaacgcgttgcgtatcgcgcggggtatcgcgtgtaattttatacggtagttggtttttggtcgtggttatcgtttttagttcgcggggttcgttacgtatacgtggtgcgatttttgtggcgattttatttggggcgtcgtgcgtaaaggtttgtagtgcgcgcgtgagtagtggtttcgcgcgtttacgagagcggaaggggtagttaaggggtagcgtagtcgtcgcgggttaagtcgcggtagagggggtcggcggggatagttttcgaggattaggttcgttattttcgttttatcgttgaagagtgcgcgaaaatggtttattttttgtcgtattttattcgtatttgggttatagatgagtagaggtggttgtttatatgtaaaaatacgttgattttaagttttttatttttaaaatgttttggttttttttgagaaagggtttgtgtttattgttttcggagtttatttttttaggtttgtttttttttaaatatttatgattttttttagaatttttagggtgaagggaaattattatttatgggagggagtttggaaaaatttagaatttttggtgggttttttgtaagtaggagttttgttgagtttttatttagtaaatatttttttttgatttagtgaattagatgttaaaatatgtacgtagttatatatttagtagtttttttgtatttttgggaatcgttagtaagtaaaggttgtttttttttgggtagatattagttggaattattaggggtgtttttatagtttttttcgttagtttggattttatcgtagatttgttgaattaattgttgggagtggattttaggtattagtaaattttaaaaattttttaaattattgtaatatggagtttgggttgagtattattgttttggtttatttaggaatttgtggatggatagtgttttaggtttgtgtgtgtatggagatttttttattcggtataagaggatattataaatttagttggggggagtataaagttgtgatagaatgtaaagaatgaataaggggtcgagcgcggtggtttatgtttgtaattttagtatttcggaaggcggaggcgggtggattatttgaggttaggagtttaagattagtttggttaatatggtgaaattttatgtttattaaaaaataaaaaaaaatgagttaggcgtagtggcgggtgtttgtaattttagttattcgggaggttgaggtgggagaattgtttgaatataggaggcggaggttgtagtgagtcgagatcgtgttattgttttttagttttggcgatagagtgagattttgttttaaaaaaaaaaaaaaaaaaaaaaagaataaggttgggatattgtagcgtttttaaagagaaataaagtagttatggagataagaagtaggatgatttgggtatgtttattagaggtagagataagggagaaattaaagataagtttgggtttttgtttttagtaattgggagtttagtggttatttttgttgtaaagaggaagttgggtaagtgtagtagtgaggttgaagaaaagggaattaaattttggttatgtttatttgaaacgttttttagatattttagtgaaggtattggtacggaggatttagtttgagggtttaggttagtgttttagtcgtggatttggggtagatgaatgtagatagattaggttagtgattaggattgagtttagattttatcgtgagatatggaagttgagttagaatttgtaaaggagttgagtaggagttgtagggggtaggaggaaaattgggagagtgtagtttttgggagttaaagggagtaagttttaaatgatgttgagggggtgagaatggagaatggaatattggattttatttggtagtatatagatcgttgaggattttgtttcgggtagttttttggaggaagaggtaagtttggttggagtgggtagaggggagagtgaaggcgaaggattagagtgtatagagattagtgttttggtttgaggggagtagagataggtgataattatagggtagacgtaggttaaaggtgtttagttttttttttaagtaaatgggtagatgtattttatatacgtttttagtgaagggtcgggtgcggtggtttaagtttgtagttttagtattttggaaggtcgaggcgggtggattatttgagattaggagtttgagattagtttggttaatatggtgaaatttcgtttttattaaaaatataaaaattagttgggtatggtggcgggcgtttgtaattttaggtatttaggaggttgaggtagaagaatcgtttgaatttaggaggcggaggttgcggtgagtcgaaatcgcgttattgtattttagtttgggtgataaaagtaagacgtagttttttgttgttgtttttttaattgttaatgaggaaaggggaagttttgtgttaggcgatagagatttaattgttgagtaggtttttttgtttgtggtttttcggtcggtttttagacgtttaggtggttaatattagagttcgcgtagtagtgtgaggtaatttattgagataggtcgggtttgcggagtttggcgagtagcggttttttttttggggtttttttttaattttcgggatattttttcgatttggagttttttcgttttatcgttaggtttttttgtagattgtaagtttatttgttattatcgttgtcgcgcgttcgtttgtttggattgttgcgggtttcgggatttgggttgggaattcgcggtggagcgggatacgaacgtggtgagcgcggggtcgagggcgtatgggaagggcgaggatgggtaggttatagtgtaggtattttcgagggttgtttgggtgtcgcgcgtaaggagcgttttaattgtcgatttttcggcggtatagagaggttaattttgcgcgggggttgggaggggagtttggattgtcggtttcgtaagtattttattcgttgtaagcggattcgggtttaggttgatttaggtttcgcgtacgcgtatttttcgtattttttcgttttcgttttcggttagaggttatttttgtgcgtttgttcggacgttggtattcgttttcgttttttgtggtaggtggggtttgtgagtggagtttcggagcgatgaggttatttttgggggcgaagcgtgcgtgttttcgtttcggcgtttttgttttaatgagataagagttagatttcggcgatttacgttttagttttaacggttgcggcgcggttttggttcgggcgtacgcgtatattgatacgcgtatacgtacgtacgcgatcggggcggtggttggcggttacggacgcgtaggattgggggacgggcgggtacggttatgggcgaggcggaggcgttttttttcgaaatgatttggagtagtacgacgagtagtggttattgtagttaagaggattcggattcggagttcgagtagtattttatcgcgcgaatttcgttagttcgtaggtcgcgtcgggattaggtgggagttagggggtgtcggcgggcgggaggggaagcggtcgttggagtttcgtttttttcggttcgttgtcgcgttttgggtcggtgggtagttttatttttttggttacgtggtttttcgcgggttttggtcggggatttgttcgcggaatcgtgcgtaagatttcgattttatcgtttagatgttgggtgtcggggtttttttggtttttgttatagataggttgaatacggaaaaagtagttgtatggtttgtggtagatttgagtcgggtattatttagttatgattaaagtcgatcgagtagtttggattagtatttcgattttcgcgttcgaatgtttttgttttttttttggggagattaggggaggatgtggagagggaagagttttcgttaggaattgagaagtatgtttaggaaaatttgagaggtagagagagattttgtttttttatttgtatttttgtatggagttagttgagtttttattttttttttgttttggtttgttattagttgttggaatgtggaagattttgttttttttttttagggtggatttggagaaagatttgggaatagataggaaagaagttttgttttggattataagtatttaggagtattttatttataggaagggggaaagttagattataaaatgtttaaagaggtggaaaaagagatttaggttattaatttaggattgtaaggtgtttcggaattttttaggtatttttattatcggagaattgtgtgttagatgttattggtgtgattattaggtttagagaattaggtttaggtattaggaaaaagaaatagggattgtgaagtttagtatgtttggtagaaatggggcggaaatttttatttaagtaaagaaagtggagttgtgagtgatgttttagataaaattttataaaattttttataaaatgggtggtgtttagtacgttaaaattttagtttagagtttgggtgtaagggttgagttgagtgtagatttttgggtttgtttttatgttagttagttttgagttattttttattgtggaaaggtgggaaaattataagatattaattaattgaaaaggagggttagttacggaggtgtatatttgtaattttagttatttgggagggtgaggtagaaggattatttgaatttgggaggtagaggttgtagtgagttaagatcgtgttattgtattttagtttgagtgatagagtgagattttgttttaaaaatagaaaaggaagttaagtacggtggtttatatttttaatgttaatgttttgggaggttaaggtaggtggattatttgtaattaggaattcgaggttagtttggttaatatggtgaaattttatttttattaaatatataaaaattagtcgggtatggtggtgtgtgattgtagttttagttatttgggagattgaattattttaatcgggaggtaaaggttgtagtgagttaagatcgtgttattgtattttaatttgggtgatagggtgaggttttgttttaaaaaaaagaaagaaggttgggtttggtgatttatgtttgtaattttagtattttgggaggttaaggtaggtagattatttgaggttaagagttcgagatttgttaggttaatatagtaaaatttcgtttgtattgaaaatataaaaaaattatttggttatggtggtgtgtgtttgtaattttagttattggggaggttgaggtaggagtattatttgaatttagaagatagaggttgtagtgagtcgagattgggttattgtattttagtttggatgagagagtaagattttgttttaaaaaaaaaaaaaaaaaaaaagaaagaataggaggttgagaagttttaagttatatgttaaaaaaaaagaaaaaaatattagttttaggttaggtgtagtggtttatatttttaattttagtattttggaaagtcgaggtgggtggattatgaggttaggagtttaagattagtttggttaaaatggtgaaatttcgtttcgattaaaaatataaaaaattagttagttgtggtggtaggtatttgtaattttagttatttgggaggttgaagtagagaattgtttgaatttaggaggtagagattgtaatgagttaagatcgtattattgtattttagtttggaaaatagagcgagattttgttttaaaaaaaaaattattagtttttatggatagtggtagagtggagggtgggtttttatggtgtagaagggaaattttatggttttgttgtgtattcgattgggatggttgttgaaatttttttttagtaggtagttttggaaatagaaaaagaaatttttttttttttagaattttggaagggttgtgtagtgtttttaatttaagtttgttttttgagtgaagatagggaggtttattattagaagggaaggggttggaaatgaggttattgtattttagtttagggtttttgggttatttaggaagggaagaaggagtaagtttttttattgttaggtaggagtttagagttattataagaataagttagtattatttttgtgttttttttgttttgtaaataaaatgatttttttttttgttttggtattagagtttgtttggtattttttttgtttttagtattttttttatttgggtattttttttcgttggtgtattgaataaatatatttattgttttatttatagtttttagtttttattttttagggtttatattatttgtttttattaattcgataaggttgtttattgtttttagtaaggtttgtattggggtttttattttagtgtttttttttatttaggagatttttggatatttggggaagaaaatgagtttaaatttttatttttttttttttattttttttttgtaaggttttggttttagtttttagttttatatttttgttggttgtagaatagtagcgggttttgggtaaggagtattttgttaaaacgttttattttgtttttttatttgttttttttatttgtttttattagatggtttaagtgtttaaggggattttagggcggagttagggagaattttggtttttttgggttaggtataagattattttataggaaattttgtgggaatttttttgggataaagtattggttagcgttgagtttagttgtgtttgtgatattcgtattttaattagggtttatttgacgttaataggaagtaaggttgatgtagtggggttaagggagtttgggagaagaaagtcggtttagagttttggttgttttgttttatattttattttttcggtaagaatttagtttttagatgaggtggggagtgagtggtcgagttaaaaatttttgggtcgggtacgatggtttacgtttgtaattttagtattttgggaggtgaaggtaggcggattatttgaggttaggagtttaagattaatttggttaatgtggtgaaattttatttttattaaaaatataaaaattagtcgggtgttgttgtggtacgcgtttgtagttttagttattcgggagtttgaggtaggagaatcgtttgaatttaggaggtagaatttgtagtgagttaagatttagttattgtattatagtttgggcgatagagtgaggtttcgttttaaaaaaaaaaaaaatttttgggttaaatttttagatagtataggtaggtgtagaaatttattaggaagttgtttgtgtatttttggtagattggagtttggtttaaagttgttttttatgtagtttgggttaaggttaaatattatgttatagtgattttttttattatgtgtgagatatggagaattggttttaagtattattttgtttattggtggttggattattgatgtgtattattttttattttttttattttgtagtgggttatggtttcgtgtcggggtagaggagaaaaatgggttgttttttttaggataaatttttattttaatttaattagggtgttgtgattagaatgtgtaattgaggtgtgattttattgattttttttttttttgagatcgagtttcgtttttgttgtttaggttggagtgcgatggtacgattttagtttattgtaatttttatttttcgagtttgagtaatttttttgttttagttttttaagtagttgggattataggtatgtgttattacgtttggttaattttgtatttttagtagagacggggtttttttatgttggttaggttggttttaaatttttgattttaggtgatttattcgtttcggttttttaaagtgttagaattataggcgtgagttaacgtgtttagtttgtttttgttttttgtgttttgaagtagggttttatttagttttttaggttggagtgtagtgatacgataatagtttattgtagttgtaatttttcgggtttaaacgatttttttattttagttttttgaatagttgggattataggtatattattatatttggttaatttttttttttttttttttagtagagatgaggttttgttatgttgtttaagttggttttaaatttttgaggattaagtgatttttttattttagttttttaaaatgttgggattgtagatgtgagttattatatttagtttgattttattttaaatgagagtttttttttagagttttttagttgtttttggtttttggttatgtgtttttagttgtttttgtttttgtggtatttttaaggttatatttagtgttgaggttttaggtaggtagtagagagaagttaaatgattttgttttttttttatttatttagagtatgtaaaattaggagtagtggtgggtttagggtgggtattagttatgtatatgtatattagggatagggggttaaaggtagttagtttttaaagattgttttagaggttattttttagagaagttttgggttttttaagggttttgtgtttatgttggtttattttgtaggacgagtttgtggagtgggagatatttgattttttttaagttgagattgagtagaagattaaggagtataatgtttagattaatagtaatttttttatgagtttggtgagttgattgtttaggaagggggcgtggggaggagtaggtatttagttatgtgtttgatatttagagggttataattgaggttattttgggtgggcgtaagtagtaatttgtgtatatttagtttagttttaagtagattgatattttatttggaatttattattaaggtttggtttttttatttttttagaataaggacggtttttatataggttttattaaggtttagttgaagttggtgcgttttgtttttgtgttttttagtaagaagttattttttttgtaggatgttcggcggggtttaggacggggtataagtgttaggcgtcgtatttttttttatttgtttaaggatgttgttaagtatttgtatgtgttgttacgtataagggtacgtgaagttattgaggttttgttgcgaaagtttttggtggtggatgattttcgtaagtttgtattttttgagcgcgttgagcgttacggttaaggtgggttttttattttattttgttttatgtgagggtatatacgtatgtatttgagtatgtaggggttgagtagttggttttgtttttgattattattttttttttatagtgtatttgcggaagttgttggatgatgagtagtttttgcggttgcggtttttggtagggtttagtgataaggttttgagttttgttttgaaggaaaatgattttggggaggtgaacgtgagtatatagtttttagttttttggttgttattagataggattgatgggttgtagttatagtaaggtttggaggaggaattgtgttggaagataagttttgtaaaatagttttaggagtgtataggtattgtaattaaagtaaaggtttttagattatttatgttaaagtttagggttgttttaagaagttaggaagaattgttttggtgttttgattttttttggtgtggaaaattttttggagatgtaggagtttatttaatgatatgaggaggttttttttagattttttatttggaagttttttggttttaaggtattaggtttgtggagtgaaattagatttagaatatgtttgatttgtttataggtaattggggaatatttgatttgg

SEQ ID NO: 16 - DNA, 3501 bp, Artificial Sequence
  chemically treated genomic DNA (Homo sapiens)
gtggggttggtaggggtgttgttttggtatagtttggggtttggtagtggtgggtggggtattggttaagagttgttattgttgtggggaggggagtttggtttgttgggattgtaggtaatgggttgtggggttttgtgggttaggaggggaatggggttgggtgggtgagtagtgggtaggggagtttagggtttggttttgggttttgttgttggatttgggggttgtgaggaagagttgtgagttgagggtttggggttggtgtattttttttgttttgtttgtagttggaaaatttttttttaagtttggggtggtggagttttgggggagaaggggttgggggagttgtggagggaggtgttgggtttgtgtgtgtagggtttaggttgaggttgggatgtgggtggggtgtaggtttgggttagggttgtagttggttgtgtgttgtgtttgtttggggtgttgtttttttttttttttgggagttgtgtggttttttttttttttttatttgttttttgttttagttttttgttttgatataatgttttttttgtgttgggtttggtttttgtgttttgtttgttatggtagttgttgtttttgttttttgtgtggttgttgtttgggttttgattgagggttgatagtttttggttagggtggtgttagggtgggtattgtgttttttttttttgtattattttttttaattggggtaattttttttgaggtgggaggtgttggttttttggttttttttttttttatttgggtaaagttttttgttttgaatgattttttttgaagtggatattttatttaaattgggtaattgtttttaaaagggttattgtgtttgaatagttttttttttggaagttttagtatttagttaggtgttttggggtgtgtaggttgttttggttttttttttattggtggttgtttattttttgtttttttttttggtttgggtgggttggtttgggtttttattttagagggtagttggttttttgttggtgtttaggttgtagggttgatgtttttgtttagttgagggaaggggaagtggaggggagaagtgttgggttggggttaggtggttagggtgttgtatggtttttatttggttggtgtgtgtttttgtaggagagtgtgttgggtagatgatgttggatatgatggaggtgtttggttattttaggtagttgttgttgtagtttaataattagtgtattaagggttttttgtgtgatgtgattattgtggtgtagaatgtttttttttgtgtgtataagaatgtgttggtggttagtagtgtttattttaagtttttggtggtgtatgataatttgtttaatttggattatgatatggtgagtttggttgtgttttgtttggtgttggattttatttatattggttgtttggttgatggtgtagaggtggttgtggttgtggttgtggttttgggggttgagttgagtttgggtgttgtgttggttgttgttagttatttgtagatttttgattttgtggtgttgtgtaagaaatgttttaagtgttatggtaagtattgttatttgtggggtggtggtggtggtggtggtggttatgtgttttatggttggttgggttggggtttgtgggttgttatgttggttatttaggtttgttatttgtttttagttgggtttttgttgttgtttgttgtggagttgtttttgggtttagaggttgtggttaatatgtattgtgttgagttgtatgtgttgggatttggtttggttgttgtattttgtgttttggagtgttgttgtttttttttttgtggtttggatttgtttaagaagagtttgttgggttttgtggtgttagagtggttgttggttgagtgtgagttgtttttgtgtttggatagtttttttagtgttggttttgttgtttataaggagttgttttttgttttgttgttgttgttgttgttgtttttttagaagttggaggaggttgtattgttttttgatttattttgtggtggtagtggtagtttgggatttgagttttttggttgttttgatgggtttagttttttttattgttggatgaagtatgagttgggtttgggtagttatggtgatgagttgggttgggagtgtggtttttttagtgagtgttgtgaagagtgtggtggggatgtggttgttttgtttggggggtttttgtttggtttggtgttgttgttgtgttattttggtagtttggatgggtttggtgtgggtggtgatggtgatgattataagagtagtagtgaggagattggtagtagtgaggattttagtttgtttggtggttattttgagggttatttatgtttgtatttggtttatggtgagtttgagagttttggtgataatttgtatgtgtgtattttgtgtggtaagggtttttttagttttgagtagttgaatgtgtatgtggaggtttatgtggaggaggaggaagtgttgtatggtagggttgaggtggttgaagtggttgttggggttgttggtttagggtttttttttggaggtggtggggataaggttgttggggttttgggtggtttgggagagttgttgtggttttattgttgtgtgttgtgtgataagagttataaggatttggttatgttgtggtagtatgagaagatgtattggttgatttggttttatttatgtattatttgtgggaagaagtttatgtagtgtgggattatgatgtgttatatgtgtagttatttgggttttaagttttttgtgtgtgatgtgtgtggtatgtggtttatgtgttagtattgttttatggagtatatgtgtatttatttgggtgagaagttttatgagtgttaggtgtgtggtggtaagtttgtatagtaatgtaattttattagttatatgaagatgtatgttgtggggggtgtggttggtgtggttggggtgttggtgggtttgggggggttttttggtgtttttggttttgatggtaagggtaagtttgatttttttgagggtgtttttgttgtggtttgttttatggttgagtagttgagtttgaagtagtaggataaggtggttgtggttgagttgttggtgtagattatgtattttttgtatgattttaaggtggtgttggagagtttttatttgttggttaagtttatggttgagttgggttttagttttgataaggtggttgaggtgttgagttagggtgtttatttggtggttgggtttgatggttggattattgattgttttttttttatttagagtgttttttgttagtttgttttgttgttgttgtgtggttttggtttgtattttagggagtggtgggggtggtgtgtagggtttattgtgtttgggataattgtagtgttgttatagtggtggttttattttttggtggttttatttggttttattgttttgtgttttagtttgg

SEQ ID NO: 17 - DNA, 3501 bp, Artificial Sequence
  chemically treated genomic DNA (Homo sapiens)
ttgagttaaggtatgaagtagtgaggttaggtgaggttgttgagaggtggagttgttattgtggtgatgttgtggttgttttgggtatagtgggttttgtgtgttgtttttgttgttttttggggtgtgggttagggttgtgtagtagtgatagagtgggttggtgaggggtgttttaggtgggagagaaatggttgatggtttggttgttgggtttggttgttaggtgagtgttttggtttagtattttggttgttttgttggggttgaggtttagtttggttgtgaatttggttagtgggtagaggttttttagtgttattttggggttgtgtaggaagtgtgtggtttgtgttagtagtttggttgtggttgttttgttttgttgttttaggtttagttgtttggttgtgaggtgagttatagtaaagatgtttttggggaagttgagtttgtttttgttgttggggttggggatgttggggagtttttttaagtttgttagtgttttggttgtgttggttgtgttttttatggtgtgtatttttatgtggttgatgaggttgtgttgttgtgtgaatttgttgttgtatatttggtatttgtagggttttttgtttgagtggatgtgtatgtgttttgtgaggtggtattggtgtgtgaattgtatgttgtatgtgttgtatgtgaagggtttgaggtttaggtggttgtgtatgtggtgtgttatggttttatgttgtgtgaatttttttttgtagatggtgtatgggtagggttgggttagttagtgtgtttttttgtgttgttgtagtgtggttgggtttttgtagtttttgttgtatgatgtgtagtggtagggttgtagtagtttttttaggttatttggagttttggtgattttgtttttgttgtttttaaaagggggttttaggttggtggttttagtggttattttggttgttttggttttgttgtatagtgttttttttttttttatgtgagtttttatgtgtgtgtttagttgtttagagttggggaagtttttgttgtatggaatgtatatgtataggttgttattgaagtttttgggtttgttataggttaggtgtgggtatgggtagtttttgaggtggttgttaggtgggttggggtttttgttgttattggtttttttgttgttgtttttgtagttgttgttgttgttgtttgtgttgggtttgtttaggttgttagggtagtgtggtggtggtgttaggttgagtgggggttttttgggtgagatggttgtgtttttattatgttttttgtagtgtttgttgggggagttgtgtttttggtttagtttgttgttatagttatttaggtttggtttgtgttttatttagtgatagaggagattaggtttgttggggtggttggggggtttgggttttgggttgttgttgttgttgtgaaatgggttggaaggtggtgtggttttttttagtttttggaagggtagtggtggtagtgatggtagggtgagaggtggttttttgtaggtggtggggttggtgttgggagggttgtttgggtgtgggggtagtttgtgtttagttagtggttgttttggtgttgtggagtttggtgggttttttttggataggtttaggttataaagaggggagtagtggtgttttgaggtatagagtgtggtggttgggttgggttttgatgtgtatagtttggtgtagtgtgtgttgattgtggtttttgggtttgagggtggttttgtggtaggtggtggtggaggtttgattggggatgggtagtaggtttggatgattggtgtggtggtttgtaggttttggtttggttgattatagggtgtgtagttgttgttgttgttgttgttgttttgtaggtggtagtatttgttgtggtgtttgaggtgttttttgtatagtgttatgaggttggggatttgtaggtagttggtggtggttagtatggtgtttaggtttggtttagtttttggggttatggttgtggttgtagttgtttttgtgttgttagttaggtggttggtgtagatgaagtttagtattaggtggaatatggttgggtttattatgttatggtttaggttgagtaggttgttatgtattattagggatttgaggtaggtgttgttggttgttagtatgtttttgtgtgtgtggaagagggtgttttgtattatgatgattatgttgtataagaagtttttggtgtgttggttgttgagttgtagtagtagttgtttggagtggttgggtgtttttattgtgtttagtattgtttgtttagtatatttttttgtggggatatatattggttgggtgagagttgtgtggtgttttggttgtttggttttagtttggtattttttttttttattttttttttttttagttgagtgggggtattagttttgtggtttgggtattggtgaaggattggttgttttttggagtgggagtttaggttggtttgtttggattaggagaaggagtaggaggtgagtggttgttggtggaggggaggttagggtggtttgtatgttttagggtatttggttgggtgttggggtttttgagaagaaaattgtttaggtgtagtgatttttttggagatagttatttgatttaagtaaaatgtttgttttaggaaaagttatttagggtggagaattttatttaagtagggagaaagggagttgaggaattagtgttttttgttttgggagaagttgttttagttgggggaagtgatatggaggaggggagtgtggtgtttgttttggtgttgttttggttgggggttgttaatttttggttggggtttgggtggtggttgtgtggggagtggaggtagtggttgttgtggtgggtagagtgtgaaggttgggtttggtgtggggagggtgttatattggggtaggaggttgaggtaggaagtaggtgggggggaggggggagttatgtagtttttaggggagggagggggtagtgttttgggtgggtatggtgtatagttggttgtggttttgatttgggtttgtgttttatttgtgttttggttttggtttgggttttatatgtgtgggtttggtgtttttttttgtggtttttttggtttttttttttttggaattttgttgttttaaatttggggaaaagttttttaattgtagatagggtgggaggagtgtgttggttttaggtttttggtttgtagtttttttttgtggtttttaaatttggtggtagagtttggagttgagttttgagtttttttgtttgttgtttgtttgtttgattttgttttttttttggtttgtggggttttgtggtttgttatttgtggttttggtgggttgggtttttttttttgtggtggtggtagtttttagttgatgttttatttgttgttgttaggttttgagttgtgttagggtagtgtttttgttagttttgt

SEQ ID NO: 18 - DNA, 2501 bp, Artificial Sequence
  chemically treated genomic DNA (Homo sapiens)
tttttatagtgtaaatgtgtttttattattttttggagtaattttatttaaaattgtttttagtataaaatttaaatatttaaatatgattttgttggttttgtttttgtggttttattttttttttttttaaatttagttagtgtttgtgttgtttgtaatgtttttttttttttgtaggggttgttattttaggttttggtttttttttagaaagttttttttttttttttttagtggggatagggtttgtttattttgatattattagtttatttatatatattggttataagtttaggttgtattgttattgaaagtttattatttgattttgagtagtttgaggattttattaaaatttaggagatgtttagtaaatgttgattgaattatgattgtttttaatatataaatgtaagattatttaggaatatttgttaaaatgtttttgttttttgagattttattttgggaggtaagtagtgggggtttaggattttgtattttgatagttttttgatgtttgtatgtagaagtgtagggattattatattgataaatttttattatttttaagggggatttttttttttaggggttatttttggaagttttttaaggataggggttgtatgttgtttttttaggttagtaattaaatttagaaaatgtttattgagtgaatgatgaaatgataggtgaatagatgaatgtaaggtgttgagttaattatttttttatataagttttagtagtttttattgtttttagttgtagaaatggtttttggaaggtaagttttttagtgagtggagttatttttaattatattttttaggattttaagggagttgtgtgttttgtgtttatttttttattagaaattggtaagttattgatttttgttttgtttttgttattttttgtttttttttgttttgtagttggtgtttagtggttttgtttgtttgtgtgtgtgttgttgtaggttttatttatgggtttattgttgaggtttgatgggtgggtggtattggttattggtgtgggggtaggtgagtatgtgaaggttggaggttgtgttttttgttgaggtgtagttggttgttttttttgggttggtatatgtgtgtagttgtagttgaggttattttgttgaggtggtggggaggggaatggttatttttgaggtattgtattttttgaggaggaaagagttggaaatatttggttttttaagtaggtatagtttgtttttttttagtattttggtgtgggttttttaaggttttgtttgagaggagaggttaggttgggttgttgattgtaaaattgggtgaaagttttttttgatttttatttgtgggtattgattgttattttttttgtaattaatttttttagatttttgtttagttttttaaaggattgaaaagttgtgaggggtgggggttggaatttgttttttgaagtgtagagatgttagtttttgaaaagttatttggttgtttagtgtttgtttttttttgttgtaagattttaagtttgtgagaggattttttttaaagagggtgtttgataagagtttttttttgttggagtttgtatgtttagtaagttataatttgtttttgaaatttattggagttttggtagaggttgtaagtttaaatgtgtataggggttaggtgtatgatggagaaagaaaatgggagtaggatgggtatatttgaggaattggagagtagagaattttgaagtggattggttagtgggaaagttgtttgtattttaggagtggtaaaatggaaaattgttatgtgaaatagttttattttttaaagtataaaaaattaaaataaattatttatattaatatagatgttgtgtagtgagattttatattagttttttattagtgggtgatttttgtaatttttaagtgtagggattttgatattatgtatttttgattttttattggtagtattttatatttggaaaggttttaatgtatgaattatttgagttatatattaaatgttataaattggaattttgttaattaatttttatgtatttttatatttgtattgataaagtggttttttatgttgttttttagaaaatgtttttagtgttgatgaatagttaagtattttatatttatagttgtttggttatttttgtatgggtatgtatttgggtgtagttatattttttaaatgtttttaggaaaatattttgtttatattttgtttttattgtaaataatgtattttataatgtttggtgttttaaattttttttgatagtttttggataatttttatgtaggaggtttagggattatattttaagatgtttttgttattgttaaggagatttttttttttaggggttatatttgaaaattatttaaggatagggattgttttttttgatattattagtatatttatatatggtatgtagtatattttatattagtatttagt

SEQ ID NO: 19 - DNA, 2501 bp, Artificial Sequence
  chemically treated genomic DNA (Homo sapiens)
attgagtattggtgtaaaatgtattgtatattatgtgtaagtatgttaatggtgttaaaagaagtagtttttatttttgaatgatttttagatatagtttttgaaaaggaaagtttttttagtgatggtaaaaatgttttagaatgtaatttttggattttttgtatgaaaattatttaagagttgttaaaaaagatttaaaatattaagtgttgtaaaatatattatttataataaaagtaaagtgtaaataaaatgtttttttaaaaatatttagaaggtatgattatatttaaatatatgtttatgtagagataattagatagttatgggtataaaatatttggttatttattaatattgaaagtattttttgaaaggtagtataagaagttattttattaatatagatatgaaagtatataggaattaattgatagaattttagtttgtaatgtttaatatataatttaaataatttatgtattagggtttttttaggtataaggtattattagtggagaattaaaggtgtataatgttaagatttttgtatttggaggttatagaggttatttattggtgagaaattaatgtaaaattttattgtatagtatttatgttggtatgaatggtttgttttaattttttgtattttaaaaaatggggttattttatataataattttttattttgttgtttttgaaatataggtaatttttttattggttggtttattttggaattttttgttttttagttttttagatgtgtttattttatttttattttttttttttattatatgtttgatttttgtgtgtatttgagtttataatttttgttaagattttagtggattttgagaatagattgtgatttgttaagtatataaattttaatggggaagggtttttattagatgttttttttaaagaaggtttttttatgaatttaaaattttatgatagagggaaataaatattgaatgattgaatgattttttaggagttgatatttttgtgttttagggggtgaattttagtttttgttttttgtggttttttagttttttaaaagattaggtaaagatttaagagagttaattgtaggaagagtaataattgatgtttatagataagggttagggagaatttttatttagttttgtaattagtagtttagtttggtttttttttttaggtaggattttgggaagtttatattggggtgttggggagaagtgggttgtatttgtttgagagattaggtgtttttggtttttttttttttaagagatgtggtgttttaagaataattatttttttttttattattttagtggggtgattttagttgtggttgtgtgtgtatgttggtttgaaaagagtagttagttgtgttttagtaaggggtgtggtttttaatttttgtatgtttatttgtttttgtgttggtgattagtattatttgtttgttgaattttagtggtgagtttatgaataaggtttgtaatgatatatatatgaataagtagagttgttggatgttgattgtgggataggaggaggtggggaatggtgggggtgggatgagggttagtgatttgttgatttttggtaggaagatgagtgtagagtgtgtggtttttttggaattttgggaaatgtagttaagagtgattttatttgttggaagatttgttttttaggggttatttttgtggttggaagtaatgggagttgttaggatttgtgtagaagaatagttaatttgatattttgtgtttatttatttatttgttgttttattatttatttaataaatgttttttgggtttagttgttgatttagagaaatagtatgtggtttttatttttgaggggtttttagagatagtttttgggaaggaaagttttttttagggatggtaaagatttgttagtgtaataatttttgtatttttatatgtaaatattaggggattgttaaaatgtagagttttggatttttattgtttattttttaaaatagaattttaaggggtaaaaatattttgataagtgtttttaaatgattttgtgtttgtatgttgagaatagttatagtttaattaatatttattgagtattttttgagttttgataggatttttaagttatttagagttagatggtaaatttttaataatggtgtagtttagatttgtgattaatgtgtgtaagtgagttaatggtgttaaaataaatagattttatttttgttggggagaaagaggaaaaattttttgaaggaggattaggatttaaagtggtgatttttgtaaagaaagaagggtattataggtagtataaatattagttaggtttggggagaaagagggtaaagttataaaagtaaagttagtaagattatgtttagatgtttgaattttgtgttgaaaatggttttaagtaggattattttagagagtggtgggaatatatttatattatggaaa

SEQ ID NO: 20 - DNA, 2470 bp, Artificial Sequence
  chemically treated genomic DNA (Homo sapiens)
aaagatgattaaaagtttaattgtttatttgaagagttgatttttttatttttgtaataaagggtatttttagtagtttttgtttattttgtttatttggttttttttgtggttgtgtaaggttataatttttgtgttttagtaaatttgtgtatgtttatttttttttttgttattatttttttttttattttgttttattattttgatgtaaaattatttgttaattttatttgaaatgagaaattttaaggtttatattatttaaattttgttagatttttatttttgttatatggtttataatgtgttgggtatttttagatttgtttattaaaaagatgtaaaataaaataatgattatttttgtggattttttttttatttttgagatgttttttttggttgtattatttttttattttttgtttattgattagaggaggggttttaattatgggtgaattttatattttattgaagaggttatgttatatgtatatttttataatataatttatatttatatagtatttttatttttagtatatttttttttattaattttaataatattattgtaagttatgttgaagtagattgtaagtgtttatttataaattgtgaaatgaattaaaatgaaagggtaaagattaaattatgattaggtttgaaattaatatataagatttaattttttttaattaaagatttttgtaggtgatttttgtttgtaggatttttttttttttttagatgttattggattgtattaggtttattgtagattttagttgttgtagaattaattagatttaagatgagttttttgattttttttggtagagttttttaattgttgaattttaatattgttgtgattagttagtgttataatttgtttgttttattttgtgtaatggattttatattatagaggtatttttttaatgttaagatgtttaagtattgtttaagtgtaaattatttaatattttttagttattaagtaattaagataggtaggattttatttgttttaaaatgatttgatttaaattaaaaagagaatgtggattttttgaattttatttggttaattttaatataatttttagtattttataattttttttaaagtttttttatttggttattttttgtattttttttgttttttttttttttttttagttataataattgttagattttgttttatttttttttgatagtttttatttttaaggttatttattttttttaggtattttttggttttagtttgagtatagtagattttaagattatatatgttatagtataggttattatagttaattttttgaataaatgtgattgaattttatgttagtaatttttatttattatttttttattaaaaaggtttaaagtttttatttaatgtttttttttatgtttattttgttaaatgattgttttttaatgatattttagaattttagaattattttattatggaggatgtgtaagattagttttttattaaataaaaagtgtgaaatggaatatgtaattttattaatttattttggttttaaaattttgtgattattagataaaatttagaaataaaatagtattattaatataaataaatttttattataattatattttttaagttttgtttgtaagaatgggtaaaatatttttaaaattttgaagaaattattatttgatagaaagtttaatttatttgtgagaaggtaaatgtatttagatataattaaagtttttttttttattttaattttatttattttgaattaagattttattgttttatttttttagatgttgttatttgaataatattgttttgagattaaaaattagtatattaatataattttttttaaatgttttaagagttttgttttttttatttttttttttaaaaataagtagttattaaattttttagtagtgaattttaaaattttttttaattttataggtttaagggtagttaaggatggttgtagttttatatgattagttgttaaagtaagttgaggtattgaagatggagaatttaaatttttgataagagttagaagataattttaattattttataaaattggaaattgaggtatttaatatgaaggtattaagattgtgatttttaattgtagtttatttatttttatttagtatttttttttgtaaatttgaggtaagatattttatttaaaagtgtattttaaattaagtaataatatgtaaatttttttttgtaaaagttagtatttatatttttaaataagatatattgaatttatttagtgaattatataaagaaaataagtgtaaaattttaatggttagttagtttttagttttttttaagattaaagagaagagattaaatatagtattattgtattgaggtaaggttttttgtgtagtttatagaaattag

SEQ ID NO: 21 - DNA, 2470 bp, Artificial Sequence
  chemically treated genomic DNA (Homo sapiens)
ttagtttttatgaattatatagaaaattttgttttagtatagtgatgttatatttggttttttttttttaattttaaaaagaattaagaattaattagttattggagttttatatttattttttttatatgatttattgaatgaatttaatatattttatttaaaaatataaatgttaatttttgtaagaaagagtttatatattattgtttaatttaaaatatatttttaagtaaagtgttttattttaagtttataagagggaatattgaataaaaatggataaattataattaaaagttatagttttgatatttttatattagatgttttagtttttagttttgtaagatgattggaattattttttagtttttgttgaagatttgagttttttatttttagtgttttaatttgttttaataattgattatatgaagttgtagttatttttggttatttttggatttataaggttaaaaaggattttgaaatttattattaaaaaatttagtggttgtttgtttttaaagaaaggggtaaaggaaataaaatttttaagatgtttaagaagaattgtgttaatatgttagtttttggttttaaaataatattgtttaagtagtagtatttaagaggatgaaatagtggagttttagtttaagataaatgaaattaaaatagaagagagaattttagttgtgtttgaatatatttgtttttttatagatggattaaattttttattaagtaataatttttttaaggttttaaagatattttatttatttttataggtaaaatttaggaaatataattatgataaaaatttatttatattagtaatattattttatttttgaattttatttgatagttatagaattttagagttagaatggattaatgagattatatattttattttatattttttatttgataaaaggttaattttatatattttttatggtgaaatagttttgaagttttaagatgttattaaaaggtaattatttaataaaatggatatgaaggagagtattaaatgaagattttaagtttttttgataggaagatggtaaataagaattattaatataaagtttaattatatttatttaaaaggttgattataatagtttatgttatggtatatgtggttttgggatttgttgtgtttaaattgaggttaaaagatatttaaagagaatggatgattttaggagtagagattgttaaagagaaatgaagtagagtttggtagttattatgattgggaaagaagaggagagataaagaagatataaaagatagttaggtaagaggattttaggaagaattatagaatgttaggagttatattaagattaattaagtaagatttaggagatttatattttttttttagtttaggttaaattattttggaataaataaaattttgtttattttaattatttaatagttaaaaagtattaagtagtttgtatttaagtaatatttaaatattttgatattaaaaaaatgtttttgtaatatgaaatttattatataaaataaggtagataggttgtaatattggttagttatgataatattggagtttagtaattggaagattttattaaaggaaattaggggatttattttagatttagttagttttataatggttagaatttatagtaaatttggtataatttaatgatatttgaggaggaaggggagttttgtaggtagggattatttataaaagtttttggttgaaaaaaattgagttttgtgtgttaattttaggtttggttatgatttaatttttgtttttttattttaatttattttataatttgtaaatgaatatttataatttgttttaatataatttatagtgatattattaggattaataaaaaaaggtatgttaaaaataaaagtattatgtaaatgtaagttatattatgaaaatatatatgtaatataatttttttagtaagatatagggtttatttatagttaagattttttttttgattaatgggtaaggggtgaagaagtaatgtagttaaaggagatattttaaaaataaaggaaaaatttataggagtgattattattttgttttatatttttttaataagtaggtttgaaaatatttagtatattataaattatatgatagaggtagggatttgatagaatttgaataatgtgaattttaaaattttttattttaaataaaattaataggtaattttatattaaaataataaaataaaataagagaaaaggtagtaatagagaaaaaaatgggtatgtataagtttattgagatatagaagttataattttatataattataaaaagagttggatgggtaagatgagtagagattgttaaaagtattttttattataggaataaaaaaattaattttttagatgaataattaaatttttaattattttt

SEQ ID NO: 22 - DNA, 7001 bp, Artificial Sequence
  chemically treated genomic DNA (Homo sapiens)
aatgtaatggaaaaagagagattgtaaagttagaaggtttaggaattgttttttgattaggtgtggaaggtaagggaaaattagtttttgaagaagatagtgagattttaatttgggtggttggagagatagtgatgttgggtatagatatggggaagttgagaggaatattatgtttgagaatggtgatttatatttgaataagtttgtaatgtttagtagattgttggaaaagtggggttggagatatatttaatggaggagttagattaatttttatttttttttatttgagagagttagtaagttatggttggaatgtgtgtgtttagtaggagagggtagggagggaagttaagagagttgggagtttgagtgaagtttttgttaaaggtagaagaggaaagttggtgtagtatagtatatttttttatttatgtttattaagtttagggataaggtttattaagatgagtttggaagagaatgttggagagaaagtggttaagaaaattgtttttattgaattttttgggttaattttgattgtaagtttttgaataattaaagtttgtgaggagatagttaatttttttattttttttatgttaatagtgaataattgtagatttttttttttttttttttttttttttttgttttttttttttttttttttgaatatttttgtttttttttgggattggtttagagtatgggtggttattgttgatttataggaggtattattgttattaataaagggtaatagtttttttttttaatatttatttatatttagtatttatttttaatattgattatggagagagtttttttgtgtttaaatattgtaatattgggggttttttaaagtataaaaatatatatttgtatgatggtattattaatatttttatggttttttattttttttttgtattggttttaagagttatttataaattttttagtaattgtatagtgttttagggttagagattggttatttttggtattgtgattagagttatttaatatttaaggtggtgattaatgtttggtaataaagtttttattgggtgttatgtgttttgggattttgagtgtgggtattttaggagtattttagtattgtgtgttagtattatggttgagagaatagttgagaaagtggttaagaggtggatttatgtgaatgttattgggaaatgagagattttgtttttaattatggttagtgtaatttgaaagtttaaaattagtttaaaataaaggtatttatttttattttatgtttatattttaggtttttaataatatgtattttttatatgtttatagaaagtagttaattgagttatttatggaaaggtttgtgggtttggttaatgaagtggaggagtattatattttagttggaaatatatttttagaatgttaaaatatttattttaaagtttggttttttggtgtaattggaggtatggtaatgtttttgtttagagattgggggttagggttagtaaggtatttgatttatatgtattttagaaggtttttattgttaaattatatttttttggaaaaattatttatgttttattttgtaaatttgatatttatatatttttgattggtattttattttagttgtaagattatgatttatagtaagtttgtttttttttttgtttggggtggtagtagaaagtatagggtattttttagtttttaagggtaggggtaaaggggttggggttttttttttttagtatagttttttttggttgtgttatattgttttttgtgagtagatagtaagtttttttttattttttattgttatttatttagtgttgtgtagtagtttagttgtgtgtttgttgggaggggttgttaagtgttttgtttattggttgttttttgaatttttgttattttatgtataaatatatttatatattttttttgtttagtttatatattgagttatttgtatatgtgagtatatttttttttttttttttatttttttggtttttgatttttataagtttatggaatatttttggaaagatgtttttgatttagtagggtaggtttgttttgatttttttttttgtagttttagtattttgagaaagtaatttatttttttggttagtgtttgtattttagtagggagatgaggattgttgttttttatgggggtatgtgtgtgtttttttttttttttaggatttgtaggattttttgtgttatttgtatataatttggtaggtttatattttttaagagttttatgaagtgttttttgtatgtgttttaaaaaggtatttgaaaattgaaagtgtgatttatggaaattaaattatttgtaaaaaattgttttggaaagtaatgattgttggttataaagggaaatatttgtgatgtatttaatgtgtttttaattttttatttgttgataatttatagttattaatgttaaatttgattttggttttagttatatttgtatattgtttaataatggtttatttttgtaagaattagataaaatgtatatttgatataaaatagttaaaaatgtaatttttagtaatagtaagtttggtatttagatagattatgaatattttgttagatattttgttgggtgtttgggatagtaattaaaataaagtattgatagttgtattagagtttattaggttgtagtaaaggaagtttatttaaaagtataaattatttaagattatagatgtatgatatattttatttattttttgtttttttaatatgtatatatatatatatatatatatatatatatatatatgtgtgtgtgtatgtgtgtgtgtatgtttaatttttaatttagttaaaaatttttttttatttgttttttatttggatatttgattttgtatattttagtttaagtgaattgagaagattgagttgtaggattaaaggatagatatgtagaaatgtattttaaaaatttgttagttggattagattgataatgtaatataattgttaaagttttggtttgtgatttgaggttatgtttggtatgaaaaggttatattttatatttagttttttgaagttttggttgtataattaatttgtggaaggtatgaatatttatgtgtgttttaattaaaggtttttttgaattattttttatatgagaatttttaatgggattaagtatagtattgtggtttaatataaatatataagttaggttgagagaattttagaaggttgtggaagggtttatttattttgggagtattttgtagaggaagaaattgaggttttggtaggttgtatttttttgatggtaaaatgtagttttttttatatgtatattttgaatttttgtttttttttttttagatgttttttgttagtttttttagttgttaaatatagttgtttgtggttggttgtgtatgtaattgtatattttattttatttgttttattttggttatagtgtagttttttttagggttattttatgtatatattatgtatttttagttaatgaggagggggaattaaatagaaagagagataaatagagatatattggagtttggtatggggtatataaggtagtatattagagaaagttggtttttggatttgttttttgtgtttattttaagtttagttttttttgggttatttttagtagatttttgtgtgtttttgttttttggttgtgaaatttagtttttatttagtagtgatgataagtaaagtaaagtttagggaagttgttttttgggattgttttaaattgagttgtgtttggagtgatgtttaagttaatgttagggtaaggtaatagtttttggttgttttttagtatttttgtaatgtatatgagtttgggagattagtatttaaagttggaggtttgggagtttaggagttggtggagggtgtttgttttgggattgtatttgtttttgttgggttgtttggttttattggatttgtaggtttttggggtagggttggggttagagtttgtgtgttggtgggatatgtgttgtgttgtttttaattttgggttgtgttttttttttaggtggtttgttggtttttgagttttttgttttgtggggatatggtttgtattttgtttgtggttatggattatgattatgattttttatattaaagtatttgggatggttttattgtattagatttaagggaatgagttggagtttttgaattgtttgtagtttaagatttttttggagtggtttttgggtgaggtgtatttggatagtagtaagtttgttgtgtataattattttgagggtgttgtttatgagtttaatgttgtggttgttgttaatgtgtaggtttatggttagattggttttttttatggttttgggtttgaggttgtggtgtttggttttaatggtttggggggtttttttttatttaatagtgtgtttttgagtttgttgatgttattgtatttgttgttgtagttgttgttttttttgtagttttatggttagtaggtgttttattatttggagaatgagtttagtggttatatggtgtgtgaggttggtttgttggtattttataggtatttgtgtttgtgttgtttgttggggtggttgttgtgtttggtaggagggagggagggagggagggagaagggagagtttagggagttgtgggagttgtgggatgtgtgatttgagggtgtgtgtagggagtttggggtgtgtggtttagtttgggggttttgtgtgtagtttgtgttgtgtttagagttaagtttttttgttgggtagttgaaaaaaatgtattttttatttatttattgtttgtgtgagaggtagatttgaaagtttgggttttttaataaaatatatgttggaaaattagataaagtagtagttatttgtgggggaaaatatttttaggtaaataaatatggggtgttttgagttatttgggaaggttttgtttttggtatttaaagttgggggtgtttggagttagtagagtttagtagagttttatttatttttttaatgtttttgtttaatgtgttttttaaatttttttttatttagattatttgattggaaatatgttagttatgatgatgattttttgggaagtgatttttgttatttgttttttttttttttttattttatgttttggggttttagagagtgattgggagttgaatgggtttgattttggagttagttggttgagtttgtgttggagtggattgttggtatgtgatttttgatagttggaaatttgtaggtgttttgtgagtttaaaataagttatatggaagtataagtgtttaaaaataattttttgttagtttagtgataagtttgttttatttggggagaatgttttggagtggtgtgtgggttagttagggtttgtgttttgtagttattgtggaaggagtgtggttggtttaggatataggagattattttgtgattttaatggtgaaggttgtgtgtttttattttaatttttttttttataagaattgttttttttttttttttttttttttttatttttttttgtttagtttttttttttgttttttgttttttgtttttttgatgggtttgtagagggattaggtgggtgtttttggtgaatatttttttaggtggttataggataggtgtattttggattgggtttggaagttttagggtgttatatggttgggttttgaattaggtattttttaattgtatattggtatttggattggtgtttttatatttttttgttttgtaagttgtggattagtttttgtttagtattttgtttttagggatatttatagtagaaggaaggggattaaagtgtagtttggttttagaggatattgaagggtagattttgggggtatttagtgtgtatttttagttgttttggagaaatttagagtattttatagttatgtagatttaagttgtttttatttaaaagataaataatgaataaaatttttaaaggttggtatattttaaattaattttatttgttttaatttagggttaaaatagagaaaaaggattttttttgtttatttttttttttttaaatggaagaataaagtatagtgattaagtttaattttatataatatttaaaattgtttgatgtgaaggaaggtattggtatgatgtgaattttataattttatgatggattttagaaattatttttttttttatttaatttttagtttttttattgtaaattaatgttgttgaattttaatgggtattaatgagattgttttttggtagattatttattgttttgttaataattataaagtgaatttggttaaatatagaggggattgtattttatttaaaattgtttattattttagtgataagtggtattagtgtaatatgttttattttatattttttgtattatatgatatttaaatatttttagaataataaaaaaagagataaggaatttaaaaattaaaaaaaaaatttgtataaatgggattttgtgtggaaatttagttttagaatgattttttttgtgttttattttttggattatttttttttttttgttagaattttgtttgttattatttagtaaggaaaagaagtatttatgtaagttttttatatggatagatattatttagtatttttttttttttagtttttttgtttaaatgattttgggtataaaggaaaggattgattgggtttttttaggaaattttaagttttttaagtagtttttaaaagttttggggttgaaagtagtgtttttaaattgtttgttatgatttagagggttatgaatttagtttagtgagtttagaatattttttaaaaggattaaaatggaaaggaatataatagaaaatattagagtgtatggtattttgtaaggataagttttgt

SEQ ID NO: 23 - DNA, 7001 bp, Artificial Sequence
  chemically treated genomic DNA (Homo sapiens)
ataaaatttatttttatgaaatattatgtattttgatattttttattatatttttttttattttagtttttttaaaaaatattttagatttattaaattgagtttatgattttttgggttatgataagtagtttgaaaatattgtttttagttttaaaatttttgagaattatttaagaaatttaaagttttttaaaagagtttaattaatttttttttttatatttagagttatttaagtagaaaaattgagaggggaaaaatattaaataatatttgtttatatgaagaatttgtatagatgttttttttttttgttggataataataggtagaattttaataaaagaggaaagataatttgggaaataaaatataggaaaaattattttaaaattgaatttttatatagagttttatttgtgtaagttttttttttaatttttaagttttttgtttttttttttattattttaagagtgtttgaatattatgtaatgtagaaagtgtaagatagggtatattatattgatattatttattattgggatgatgaataattttgaataagatgtgattttttttgtatttgattaggtttattttgtaattattagtaaggtagtaaataatttattaaggagtagttttattagtgtttattgaaatttagtagtattaatttgtaataaaagaattgaaaattaaatagggaagaaaatggtttttggagtttattataaggttatggaatttatattatattagtgtttttttttatattaagtagttttaaatgttgtgtggaattagatttaattgttgtattttgtttttttatttaaaaaaaaaaaggtgggtagaagaaattttttttttttgttttaattttaaattaaaataagtaaaattaatttgaaatatgttaatttttaaaagttttgtttattgtttgttttttgagtaaagatagtttggatttgtgtggttgtgggatgttttaaatttttttaaggtggttgaagatgtatattgaatatttttagaatttgttttttagtattttttggggttaaattgtattttagtttttttttttttgttataaatatttttggaaatagaatattgaataaaaattggtttatggtttataaggtagaaagatatagggatattagtttggatattagtgtatagttgggaaatgtttaatttaggatttagttatgtggtgttttgaagtttttaaatttagtttggggtatatttgttttgtggttatttaggaaggtgtttattagaagtgtttatttaatttttttgtaggtttattaggaaaataaaaaataaaaaataaaaggagaaattgggtaagagaaaatgggagggagaggagagggagaaagaataatttttgtagggaaaaaaattaaaatgaggatatataatttttgttattgaagttataaagtggttttttgtgttttggattggttgtgttttttttatagtggttgtgaggtgtagattttggttgatttgtatgttattttggggtattttttttgggtgggataggtttgttattgggttggtaggagattatttttaagtatttgtgtttttatatggtttgttttaaatttgtgggatatttataaatttttggttgttagaagttatatgttagtaatttgttttagtgtggatttagttagttaattttgaaattagatttatttaatttttaattgttttttaaagttttaggatgtggggtggggaggaggggaaagtgggtgataggaattgttttttagaaagttattattatagttgatatatttttaattaaatagtttagatgaaaggaaatttggggagtatattaaataaaaatattaaaaggataaataaaattttgttgagttttgttaattttaaatatttttaattttaaatgttaagagtgagatttttttaagtgatttaaagtgttttgtgtttatttgtttggaggtgtttttttttataaataattgttgttttgtttggttttttaatgtgtgttttgttaggaagtttgggtttttgggtttgttttttgtatggatggtaagtgggtggagagtatgttttttttagttgtttggtgagagaatttgattttgaatgtagtgtgggttgtatgtagaatttttgggttgggttgtgtgttttgggttttttgtgtgtatttttgggttgtgtgttttgtggtttttgtagttttttaggttttttttttttttttttttttttttttttttttgttgggtgtggtggttattttgatgggtggtgtgggtgtgggtatttgtagaatgttggtgggttggttttgtgtattgtgtagttgttgggtttgttttttaggtagtagggtatttgttggttgtggggttgtaggaaaggtgatagttgtggtggtgggtgtagtagtattagtgggtttggagatatgttgttgagtggggggaaattttttaggttgttggagttgaatgttgtagttttagatttggggttgtaggggaggttggtttgattgtagatttgtgtgttggtggtggttgtggtgttgaatttgtaggtggtgtttttggggtagttgtatatggtgggtttgttgttgtttaggtatattttgtttaggggttgttttagggggattttgagttgtggatggtttaggggttttagtttgtttttttggatttgatgtagtagggttattttagatgttttggtgtggagggttatggttatggtttgtggttgtgggtagggtgtagattgtgtttttgtagggtagaaggtttagaaattggtgggttatttggaaaaagagtatagtttgaggttagaggtgatgtagtgtatgttttgttgatatgtgagttttggttttggttttgttttgggagtttgtgggtttggtgaagttgggtgatttgatgggagtaagtgtagttttaggatgaatgttttttgttagtttttgggtttttgggtttttaattttaagtattggttttttgagtttatatgtattataaaggtgttggaggatggttagggattgttgttttgttttgatattggtttaaatattattttaggtataatttgatttggagtgattttaaagagtagtttttttgaattttattttatttgttgttgttgttggatagaggttgagttttatggttagggggtgggggtgtatgaggatttgttaaaggtggtttagggaagattgggtttaaaataaatgtgaaagatggatttaggggttggttttttttaatgtgttgttttatgtgttttgtgttagattttgatatatttttgtttgtttttttttttgtttgattttttttttttgttggttagaaatatgtagtgtgtatataggatgattttggggaggattatattgtaattgagatagggtagatagaatggggtgtgtggttgtatatgtagttagttatagatagttatatttagtagttgggggaattgatagggggtatttgaggggaagggggtggagatttagggtatatatataggaagagttgtattttgttattaggagaatgtaatttgttaggattttagtttttttttttgtaaaatgtttttaaagtagatagatttttttataattttttgagatttttttagtttgatttgtgtgtttatgttggattatagtattgtatttggttttattaggaatttttatgtgaaggatgatttagaaaaatttttggttagggtgtatatgggtgtttatgttttttataggttggttatgtaattaaaattttagaaaattgaatataaaatgtgatttttttatattaaatataattttaggttatgaattaaagttttggtaattatgttatattgttggtttggtttagttaatagatttttaaaatgtatttttgtatgtttattttttagttttataatttgatttttttggtttatttgggttaggatatgtagaattaaatatttagatgaaaaataaatagaaaaaagtttttaattgaattaaaagttaaatatgtatatgtatatatatatatatatatatgtgtatatatatatatatatatatatatatatatattaaggagataaaaaataggtgaagtatattatgtgtttataattttggatagtttatatttttgaataaattttttttgttgtagtttaatagattttgatataattattaatattttgttttaattgttattttaaatatttaatagagtatttgatgaagtgtttatggtttatttaaatgttaagtttattgttattaagagttatatttttgattattttatattaagtatatattttatttaatttttataaaaatagattattgttggataatatgtaaatgtagttgaagttaaaattgagtttagtattaatgattatagattgttagtaaataaagggttaaaaatatattaggtgtattgtagatatttttttttatggttagtaattattattttttaaagtaattttttatagatgatttaatttttataaattatatttttaatttttaaatgtttttttaaaatatatgtaaaaagtattttatagggtttttaaaaaatgtgaatttgttaaattatatgtaaatggtataaagaattttataagttttgaaagaaaaaggagatatatatatatttttatggagaatagtaatttttatttttttgttaggatatagatattagttagaaaggtaagttgtttttttaaaatgttaaagttatagagagagaaattaaaataagtttattttgttggattaagaatgtttttttagaaatgttttatgggtttgtagaagttaagggttgagagagtgagaaggaaggaaggaatgtgtttgtatgtgtgagtggtttagtgtgtgaattaggtagagagagtgtgtggatgtgtttgtgtgtggaatggtagggatttgggaagtagttagtaggtagggtatttggtagttttttttggtagatatgtagttgggttattgtatagtgttggatgaatggtagtggggagtgaggggagatttgttgtttgtttatagggagtagtgtggtatagttagagaaagttgtattggggaggagaaattttagtttttttgtttttatttttggaggttggaaagtattttatgttttttgttgttattttaagtaagaggaaaaataggtttgttgtgaattatagttttatggttaaaatagaatgttagttaaaagtgtatggatattaagtttataaaataggatatgggtggtttttttgaaagaatataatttaataataaaagttttttgggatatatgtggattaaatgttttattggttttagtttttagtttttgaatagaggtattgttatgtttttgattgtattaggaaattagattttggaataaatgttttggtattttagggatgtgtttttagttgaaatgtaatatttttttattttgttaattaaatttataaatttttttatgaatagtttagttgattgttttttgtaaatatgtgaaaaatatgtattattaaaagtttaggatatgaatataagataaaggtagatatttttgttttaaattgattttaggtttttgagttgtattgattgtgattgggaatgaggttttttattttttagtggtgtttatatggatttattttttgattatttttttaattatttttttggttatagtattaatatgtaatattgaggtgtttttagagtgtttatgtttagggttttaggatatatgatatttaatggaggttttgttgttagatattagttattattttggatattaaatgattttaattataatgttaggagtggttggtttttggttttgggatattatgtagttattgagagatttatgagtggtttttgagattagtataaaaaagaaatagaaagttataaaaatgttaatgatgttattatgtaaatatatgtttttgtgttttgaaagatttttagtattgtagtgtttgagtataggagagttttttttatagttagtattgaaaataaatattggatataaataaatattgaaaagaaagattgttattttttgttggtgatagtggtgttttttgtaggttaataatggttatttatgttttagattagttttagaaaaaagtaagagtatttagggagggaggagagaggaataggggaaaggagaaggaaaggaaaggggatttgtaattgtttattattgatataggaagaataagaaggttagttgtttttttataggttttgattgtttagagatttataattaaagttagtttaagaagtttagtaaaggtagtttttttaattattttttttttagtatttttttttaaatttattttggtgagttttgtttttgggtttggtgagtatgggtgggaaagtatattgtgttatgttgattttttttttttgtttttggtaaaaattttatttgggtttttagtttttttggtttttttttttatttttttttgttggatatatatgttttagttgtgatttattggtttttttaggtgaagaagggtaaagattgatttggtttttttgttgaatgtgtttttagttttatttttttagtggtttgttgggtattgtaggtttgtttaaatatgagttattatttttaaatatggtgttttttttaatttttttgtgtttgtgtttagtattattgtttttttagttatttagattaaaattttattgttttttttgagggttgatttttttttgttttttatatttaattaagaggtaatttttaagttttttagttttataatttttttttttttattgtatt

SEQ ID NO: 24 - DNA, 11001 bp, Artificial Sequence
  chemically treated genomic DNA (Homo sapiens)
ttaagttagatgttttttaattatttgtggataggttaggtatattttgagtttaattttattttataggtttaatattttggagttagaaagtttttaggtaaaaagtttgaagggggtttttttatgttattagatggatttttgtatttttagaagattttttatattaggaaagattaaagtattaaggtaattttttttggttttttgggataattttaggttttggtatgagtggtttggaagtttttgttttagttataatgtttatatatttttggaattgttttgtagggtttgttttttagtataattttttttttaagttttattgtagttatagtttattagttttgtttagtgataattaagaaattaagaattatgtatttatgtttatttttttagagttatttttttttaggataaagtttagggttttgttattgggttttgttaggagttgtagttgtaggggttgtttattatttaatagtttttgtaagtatattgtgaaggggaagtaatgattagagatagggttagttgtttagtttttgtatgtttaggtgtatgtgtatatatttttatatagggtagggtggggtgggaagtttattttggttgtgatgtttagtgtgtttaaagagtgtaaatttgtgggggttatttattattaagaatttttgtagtagggttttaatgattttatgtgtttttgtgtgtgatagtatatgtaggtgtttgatagtatttttgggtaggtaaaaggaagtgtggtgtttgatatttgtgttttgttttgggttttgttgggtattttgtaaggagggtggttttttgttggagggtatagagatagggtgtattagttttagttgaattttgatgaagtttgtgtaagaattgtttttgttttaaagaaatagagaaattaaattttgataataggttttaggtgagatgttagtttatttggggttaggttgggtatgtataaattattgtttgtgtttatttaagataattttagttgtgattttttgagtattaggtatatagttgggtatttgtttttttttatgttttttttttgagtagttaatttattaagtttatgaagaggttgttgttgatttgggtattgtattttttgattttttgtttaattttagtttgagaaaggttaggtgttttttattttataggtttgttttgtaagatgggttagtatggatatagggtttttgaggaatttagggtttttttgaaaaatggtttttggggtagtttttggaaattgattgtttttggttttttgtttttgatgtatatatatatagttggtgtttattttgaatttattattgtttttggttttgtatgttttgggtggataagggaaagatagaattatttggtttttttttgttgtttgtttagggttttagtattgaatgtagttttaaggatattatagaagtaggggtaattgaaggtatatggttaggggttaggaatagttgagggattttgaagagggatttttatttaaagtaaaattaggttgggtgtggtggtttatatttgtaattttagtattttgggaggttaaggtaggaggattatttgatttttaggagtttgagattagtttgggtaatatagtaagattttatttttattaaaaaaagaaaaaaaaaaattagttaggtgtggtggtgtgtttgtagttttaattgtttaggaggttgaggtgggaggattgtttgagtttgggagattgtagttatagtaagttattattgtgttattgtattttagtttggggaattgagtgagattttgttttaaaatataaaaaataaaaataggttgggtatgttggtttatgtttgtaattttagtattttgggaggttgaggtgggtggattatttgaggttaggagtttgagattagtttgattaatatggagaaattttgtttttattaaaaatataaaattagttaggtgtggtggtatatgtttgtaattttagttatttaggaggttgaggtaggagaattgtttaaatttgggaggtggaggttgtagtgaattgagattgtgttattgtattttagtttgggtaataagagtgaaatttggttttaaaaaaaaaaaaaattagtaaaattatattttaattgtatattttgattatagtattttagttgagttggagtgagggtttgttttggagaaggtagtttattttttttttttgttttggtatggggttatgatttattgtagggtgagaggagtggagagtggtgtatattagtagtttagttattagtggatagagtagtatttggagttagttttttatgttttatatatagtgagaaaaattattgtgatatgatgtttaattttgatttaagttgtataaaaggtagttttaggttaggttttaatttgttagaggtatataggtagttttttggtgggtttttgtatttgtttgtgttgtttggagatttggtttaaagattttttttttttttgagatgaagttttattttgttgtttaggttgtagtgtagtggttggattttggtttattgtaagttttgttttttgggtttaagtgatttttttgttttagatttttgagtagttgggattataggtgtgtgttataataatatttggttaatttttgtatttttagtagagatgggattttattatattggttaggttggttttgaatttttgattttaagtgatttgtttgtttttattttttaaagtgttgggattataggtgtgaattattgtatttgatttagagatttttaatttgattatttattttttattttatttagggattggatttttgttggaagggtggagtgtgggatagggtagttagggttttgaattgattttttttttttagatttttttggttttattgtattagttttattttttgttgatgttagataggttttagttagaatgtgagtgttatagatatagttaagtttagtgttgattaatattttgttttagaagaatttttataaggttttttgtagaatgattttgtgtttagtttaggagagttagggttttttttgattttgttttggagtttttttaagtatttaaattatttgatggggataaatggagaggatagatgagggagtagggtggagtgttttagtagaatgttttttatttagaatttgttgttattttgtagttagtaaggatgtggggttaagaattaaggttagggttttataggaaaaaggtaaagggggaggggtgggaatttaagtttatttttttttttaagtatttaaaggttttttggatggagaagagtattggagtaaaaattttagtataaattttattggggatagtgggtaattttgttgggttagtaaaaataaatggtgtgggttttggaaaatgagggttggaggttgtgaataaagtagtggatgtgtttgtttagtatattaatgggaagaagtatttagatgggaggagtattaggggtaggagaaatgttagatagattttagtgttagggtaagaaggaagattattttgtttgtagaatagggagggtatagggatggtgttaatttgtttttgtgatggttttgagtttttatttaataatgagaaagtttgtttttttttttttttttggatgatttaggagttttgggttgggatgtagtgattttatttttagtttttttttttttggtgatgaatttttttatttttatttagaaaatagatttggattagaggtattgtatagtttttttaggattttaaaggaggaagagtttttttttttgtttttaaagttgtttgttggaagaggattttaatagttattttagttggatgtatagtaggattatggaattttttttttgtattatagggatttattttttattttattattgtttataaaaattgatggttttttttttgagatagagttttgttttgttttttaggttggagtgtagtggtgtgattttggtttattgtaatttttgttttttgggtttaagtaattttttgttttagttttttaagtagttgggattataggtgtttgttattataattggttaattttttgtatttttagttgagatggggttttattattttggttaggttggttttgaatttttgattttatgatttatttattttggttttttaaagtgttgggattaaaggtgtgagttattgtatttggtttaaaattgatgtttttttttttttttttaatatataatttgggattttttagttttttattttttttttttttttttttttttttttgagatagagttttgtttttttatttaggttggaatgtagtggtttagttttgatttattgtaatttttgttttttgggtttaagtgatatttttgttttagtttttttagtagttgggattataggtatatattattatggttagataatttttttgtatttttagtatagatggggttttgttatgttggtttggtaggttttgaatttttggttttaagtgatttgtttgttttggttttttaaaatgttgagattataggtatgagttattaagtttagtttttttttttttttttgagatagagttttattttgttatttaggttggagtgtagtggtatgattttggtttattgtaatttttgttttttggttgaagtgatttagttttttaagtagttgggattatagttatatattattatgtttggttaatttttgtatgtttagtagagatagggttttattatgttggttaggttgattttgaatttttgattgtaaatgatttatttgttttggttttttaaagtattggtattagaggtgtgagttattgtatttggtttttttttttatttttgagatagagttttattttgttatttaggttggagtgtagtggtatgattttggtttattgtaatttttgttttttaggtttaagtgatttttttgttttatttttttaagtagttgggattataggtgtgtatttttgtggttagtttttttttttaattggttagtgttttgtggtttttttatttttttatagtggaaaatggtttaggattgattgatatgaagataagtttaggggtttatatttaatttaatttttgtatttaagttttgggttaagattttggtgtgttgagtattatttattttgtaaggaattttgtaaaattttatttgaagtattatttataattttattttttttatttaaataaggatttttgttttatttttgttaggtatattgagttttatagtttttgttttttttttttggtgtttaggtttggttttttgagtttggtggttatattaatggtatttggtatatagttttttgataatggggatatttaggaggttttgagatattttatagttttgggttagtaatttggatttttttttttatttttttaggtattttataatttagttttttttttttttgtgggtaaagtgtttttgaatgtttatggtttaaaataagattttttttttatttatttttaaatttttttttagatttattttagaggaagggaatagaattttttatattttagtagttggtgataggttagaatagggaagaggtgagggtttagttggttttatataggagtgtagatggaggagtaggatttttttttgttttttaagtttttttaaatatattttttaatttttggtgaggatttttttttttttatatttttttttagtttttttaaggagggagtaggagtatttgaatgtggaaattgaggtgttagtttaaattgtttggttggttttagttatagttggataatgtttggtttaggtttattataagttatatagttgttttttttgtgtttaatttgtttgtgatagaaattaagggggttttggtatttagtatttaggtggtggaattggggttttatgtatggttttgtgggtaggtttttggttaggatttgtggggagttatgtagttaggagggtggggttgtttattgatttaggatgtggtaatggattggggagggtggagttttagtgattgtttttttttttgtttgttggtattttttggtttttatttggttttggtgtggtttgtgagttagtgaggtttgtgtggtgaagtattgtttgagttttgagtttgagtttttttggttgtagtagttattgtttgttgtgttgttttaggttattttgaaagaaggtgtttttgttttgtttatagttgtatttgtttgttttttagttttgtgtgtttgtagttgttaattattgttttggttgtgtgtgtgtgtgtatgtgtgttagtgtgtgtgtgtgtttgggttagagttgtgttgtaattgttaagattgaaatgtagattgttgggatttagtttttgttttattggggtaggaatgttggggtggggatatgtatgttttgtttttaggaatgattttattgttttggagttttatttatagattttatttattatagggaatgggggtgggtgttagtgtttgggtaagtgtataagagtggtttttggttggaggtgagggtgggaaggtgtgggaagtgtgtgtgtgtggagtttgggttagtttgggtttgggtttgtttgtagtgggtggagtatttgtggagttggtaatttaggttttttttttagtttttgtgtagaattagtttttttgtgttgttgggaaattggtaattagaatgttttttgtgtgtggtatttaggtagtttttgagaatgtttgtattgtggtttgtttatttttgttttttttatatgtttttggttttgtgtttattatgtttgtgttttgttttattgtgggtttttagtttaggttttggggtttgtaatagtttaggtagatgagtgtgtggtagtggtagtggtaggtgaatttgtaatttgtagagaggtttggtggtgaggtggaggagttttaggttggggaaatgttttggagattgaagggaagttttagggagagggttgttgtttgttaggttttgtaggtttgatttattttagtgggttattttatattgttatgtggattttaatgttggttatttgggtgtttggaaattggttggaaggttataggtagagaggtttgtttaatagttggatttttattgtttagtatagaattttttttttttttattggtaattaaaaaaataataataaaaaattgtgttttgtttttgttatttaggttggagtgtaatggtgtgattttggtttattgtaatttttgttttttgggtttaagtgatttttttgttttagttttttgagtatttaggattataggtgtttgttattatgtttagttaatttttgtatttttagtagagatggggttttattatgttagttaggttggttttaaatttttgattttaggtgatttatttgttttggttttttaaagtgttgggattataggtttgagttattgtatttggttttttattgggaatgtatatggaatatatttgtttatttatttgaaggaaaaattaaatatttttaatttatgtttgttttgtggttgttatttgtttttattttttttagattaagatattggtttttatatattttaattttttgtttttatttttttttttatttattttagttaggtttgtttttttttttaggaaattgtttgggatagggtttttagtgatttgtgtattattaaatggaatttagtgttttattttttatttttatttttttagtattatttgaagtttgttttttttgatttttaggggttatatttttttagttttttttttattttttgtagtttttgtttagtttttttgtagattttgatttaatttttatattttatgatgaagtttgggtttagttttgattattggtttggtttgtttatatttatttgttttagatttatggttgaaatattgatttaaatttttagattagattttttgtgttagtatttttattaggatgtttaaaagatgttttaagtgaatatggttaaaatttaatttttttttttttagttttattgttatatttgtttagttttttttttgtagtaaaaatggttattaggtttttagttattggagataaaagtttaaatttatttttgattttttttttgtttttatttttgataaatatgtttaaattattttgttttttatttttatggttattttatttttttttgagaatgttgtaatgttttagttttgttttttttttttttttttttttttttgagatagagttttattttgttgttaaggttggagggtagtggtatgattttggtttattgtaatttttgttttttgtgtttaagtaatttttttattttagttttttgagtagttgggattataggtatttgttattatgtttggtttatttttttttattttttagtagatatgaggttttattatgttggttaggttggttttgaatttttgattttaggtgatttatttgtttttgttttttgaagtgttgggattataggtatgagttattgtgtttggttttttgtttattttttgtattttgttataattttgtgttttttttagttgaatttgtgatgtttttttgtattggatgagagggtttttatgtatatatagatttgggatattatttatttataagtttttaaataggttagagtagtgatgtttaatttagattttatgttataataatttggggagtttttaaaatttattgatgtttagggtttatttttagtagttgatttaataggtttgtggtgggatttaggttagtggggaggattgtaaaagtatttttggtgattttagttggtgtttatttaggggagagtaatttttgtttgttggtgatttttaggggtgtagaaggattgttgggtgtgtggttgtgtgtatattttagtatttgatttattgggttagaaaagggtgtttgttaaataaagatttaataaaatttttgtttgtagggggtttattaaaggttttaaatttttttaggtttttttttataggtggtaatttttttttattttaaaggttttggagggggttatgagtgtttgagaagaggtaagtttgggaagatggattttgaggatagtaggtataaatttttttttaagaagggttaaggtattttaaagataagaaatttaaaattagtgtatttttatatataagtagttatttttgtttatttgtggtttagatatgagtggagtgtgataagggataaattatttttgtgtattttttagtgatggggtgaaagtaatggatttagtttttgggagttgtttttgttgattttttttgttgtgatttgatttgtggtgattgtgttgttttttggttgttttttttgtttttgtaggtgtgtggggttattatttatgtgtgtattgtaggtttttgtgtatgatgttttagatgaagttgttatagaggttgtattatgtgtgtgtggtgggttttgtgggttggaagtggtggttatggttagggattagttgttgtgtggggttgtatgtggtgttttgtgtgatgtgtagtgtgttggtatgttttagttgggtgtggttttttttagtgtgtttagtgggtgttagtttttgtagtttaatgagtttaggtttttttgatatggtttggttgggtttgtgttttgttggttttgggtgttagtaagtgtgggttgggtggggttatagggtgggttttgattttagtgttttttttaggatttagattgggtggtgggaaggagttgaggagagttgtgtaatggaaatttgggtgtagggattgtggggtttgaaggtggggttgggtgtgtttttgtagagttttttttgttttgttttttttttttttttttgttttttttttatattttattttggatggttataatgatggtgattgtaaagtattatgtggagatatttgtgtttttggaggttagttttattgtgttagaggaagagggtttttatatttggttttggtttttttggtttggtttgttgaagtaatatatttggtttatttattgggtggggtaggaagttttgagtttttatttggggtgaggaggagggagattggttagtagttttattgtttgttttgttttttattgtggagattggggttttggtagaggttggattgtgattttgaggtttaggggtgtattttgggtggatttttttggtatgggtggttggtttttagtaattgtagtttttatttggttttgttattttgggttgttaggatataagtttttttatgttttttttagtgtttgatttggtattttttgtaggtaggtgggtattgaggatggtaatgtatgtgggggatgtgggagtagggtttagaggtttaaggttttaggatatttttatttgtagtaatattatttattttggtattgtgagtagtgtttagaagtttttgtattgtagtaagtatagtggggttgttttggagttattgtttttagtatatttagtttgtaggttttagtttatttgggggaaagttaggaaggtttgattggttttggaaggtgggggtattttatttatatttatgttttttgtattttttttattttttttgttatttttataggttttatttttgtgtttgtagttgtaggttttgttttgaggggttgaatatatgttggagttggtgtttggtaattgtttgttatttgtttttgtttttttgttttagttgtttttagatttttgggatttagga

SEQ ID NO: 25 - DNA, 11001 bp, Artificial Sequence
  chemically treated genomic DNA (Homo sapiens)
ttttagattttagaaatttgggagtggttggagtgagaaaatagaggtaagtggtaggtaattgttaagtattagttttagtatgtgtttagttttttagagtaggatttgtggttgtaggtgtgaaggtaaggtttgtggaaatggtagggagggtggaggggatgtaggaggtatggatgtgggtggggtgtttttattttttagggttagttagatttttttgatttttttttaggtgggttgagatttataggttggatgtgttagaggtagtggttttagagtggttttgttgtgtttattgtagtgtagaggtttttaagtgttgtttatgatgttagaatgagtggtattgttgtaggtgagggtattttagaattttggatttttaagttttatttttatattttttatatgtattgttatttttaatatttatttgtttgtagggagtgttaagttaagtattgggaaaagtatggaaagatttgtgttttggtagtttagggtgatagagttaaatgagggttgtagttgttgagggttgattatttatgttaagggaatttatttagaatgtatttttgaattttaagattatggtttagtttttgttggagttttagtttttgtagtggagagtagagtgggtggtaaagttgttgattgattttttttttttttattttaagtgaaggtttgagattttttgttttatttagtgggtaggttaagtgtgttgttttagtaaattggattaggagggttagggttggatgtggggattttttttttttagtatagtaaagttggtttttagaaatatgggtatttttgtgtggtgttttgtggttgttgttgttgtggttgtttggggtggggtgtgaggaggggatgaaggagggaaggaagggtaaggtggggggggttttgtgagagtgtgtttagttttgtttttgggttttatagtttttgtatttaggtttttattgtgtggttttttttagtttttttttgttgtttagtttggattttgggggaggtgttgaagttggggtttgttttgtggttttgtttggtttgtgtttgttagtgtttaaagttagtgaagtatgggtttaattgggttatgttgggggagtttgagtttattgagttgtgggagttggtatttgttgggtgtgttgggaagggttgtatttggttggagtgtgttaatgtgttgtgtattgtgtggggtattgtgtgtaattttatatggtagttggtttttggttgtggttattgtttttagtttgtggggtttgttatgtatatgtggtgtgatttttgtggtgattttatttggggtgttgtgtgtaaaggtttgtagtgtgtgtgtgagtagtggttttgtgtgtttatgagagtggaaggggtagttaaggggtagtgtagttgttgtgggttaagttgtggtagagggggttggtggggatagtttttgaggattaggtttgttatttttgttttattgttgaagagtgtgtgaaaatggtttattttttgttgtattttatttgtatttgggttatagatgagtagaggtggttgtttatatgtaaaaatatgttgattttaagttttttatttttaaaatgttttggttttttttgagaaagggtttgtgtttattgtttttggagtttatttttttaggtttgtttttttttaaatatttatgattttttttagaatttttagggtgaagggaaattattatttatgggagggagtttggaaaaatttagaatttttggtgggttttttgtaagtaggagttttgttgagtttttatttagtaaatatttttttttgatttagtgaattagatgttaaaatatgtatgtagttatatatttagtagtttttttgtatttttgggaattgttagtaagtaaaggttgtttttttttgggtagatattagttggaattattaggggtgtttttatagttttttttgttagtttggattttattgtagatttgttgaattaattgttgggagtggattttaggtattagtaaattttaaaaattttttaaattattgtaatatggagtttgggttgagtattattgttttggtttatttaggaatttgtggatggatagtgttttaggtttgtgtgtgtatggagatttttttatttggtataagaggatattataaatttagttggggggagtataaagttgtgatagaatgtaaagaatgaataaggggttgagtgtggtggtttatgtttgtaattttagtattttggaaggtggaggtgggtggattatttgaggttaggagtttaagattagtttggttaatatggtgaaattttatgtttattaaaaaataaaaaaaaatgagttaggtgtagtggtgggtgtttgtaattttagttatttgggaggttgaggtgggagaattgtttgaatataggaggtggaggttgtagtgagttgagattgtgttattgttttttagttttggtgatagagtgagattttgttttaaaaaaaaaaaaaaaaaaaaaaagaataaggttgggatattgtagtgtttttaaagagaaataaagtagttatggagataagaagtaggatgatttgggtatgtttattagaggtagagataagggagaaattaaagataagtttgggtttttgtttttagtaattgggagtttagtggttatttttgttgtaaagaggaagttgggtaagtgtagtagtgaggttgaagaaaagggaattaaattttggttatgtttatttgaaatgttttttagatattttagtgaaggtattggtatggaggatttagtttgagggtttaggttagtgttttagttgtggatttggggtagatgaatgtagatagattaggttagtgattaggattgagtttagattttattgtgagatatggaagttgagttagaatttgtaaaggagttgagtaggagttgtagggggtaggaggaaaattgggagagtgtagtttttgggagttaaagggagtaagttttaaatgatgttgagggggtgagaatggagaatggaatattggattttatttggtagtatatagattgttgaggattttgttttgggtagttttttggaggaagaggtaagtttggttggagtgggtagaggggagagtgaaggtgaaggattagagtgtatagagattagtgttttggtttgaggggagtagagataggtgataattatagggtagatgtaggttaaaggtgtttagttttttttttaagtaaatgggtagatgtattttatatatgtttttagtgaagggttgggtgtggtggtttaagtttgtagttttagtattttggaaggttgaggtgggtggattatttgagattaggagtttgagattagtttggttaatatggtgaaattttgtttttattaaaaatataaaaattagttgggtatggtggtgggtgtttgtaattttaggtatttaggaggttgaggtagaagaattgtttgaatttaggaggtggaggttgtggtgagttgaaattgtgttattgtattttagtttgggtgataaaagtaagatgtagttttttgttgttgtttttttaattgttaatgaggaaaggggaagttttgtgttaggtgatagagatttaattgttgagtaggtttttttgtttgtggttttttggttggtttttagatgtttaggtggttaatattagagtttgtgtagtagtgtgaggtaatttattgagataggttgggtttgtggagtttggtgagtagtggttttttttttggggtttttttttaatttttgggatatttttttgatttggagtttttttgttttattgttaggtttttttgtagattgtaagtttatttgttattattgttgttgtgtgtttgtttgtttggattgttgtgggttttgggatttgggttgggaatttgtggtggagtgggatatgaatgtggtgagtgtggggttgagggtgtatgggaagggtgaggatgggtaggttatagtgtaggtatttttgagggttgtttgggtgttgtgtgtaaggagtgttttaattgttgattttttggtggtatagagaggttaattttgtgtgggggttgggaggggagtttggattgttggttttgtaagtattttatttgttgtaagtggatttgggtttaggttgatttaggttttgtgtatgtgtattttttgtatttttttgtttttgtttttggttagaggttatttttgtgtgtttgtttggatgttggtatttgtttttgttttttgtggtaggtggggtttgtgagtggagttttggagtgatgaggttatttttgggggtgaagtgtgtgtgtttttgttttggtgtttttgttttaatgagataagagttagattttggtgatttatgttttagttttaatggttgtggtgtggttttggtttgggtgtatgtgtatattgatatgtgtatatgtatgtatgtgattggggtggtggttggtggttatggatgtgtaggattgggggatgggtgggtatggttatgggtgaggtggaggtgtttttttttgaaatgatttggagtagtatgatgagtagtggttattgtagttaagaggatttggatttggagtttgagtagtattttattgtgtgaattttgttagtttgtaggttgtgttgggattaggtgggagttagggggtgttggtgggtgggaggggaagtggttgttggagttttgttttttttggtttgttgttgtgttttgggttggtgggtagttttatttttttggttatgtggttttttgtgggttttggttggggatttgtttgtggaattgtgtgtaagattttgattttattgtttagatgttgggtgttggggtttttttggtttttgttatagataggttgaatatggaaaaagtagttgtatggtttgtggtagatttgagttgggtattatttagttatgattaaagttgattgagtagtttggattagtattttgatttttgtgtttgaatgtttttgttttttttttggggagattaggggaggatgtggagagggaagagtttttgttaggaattgagaagtatgtttaggaaaatttgagaggtagagagagattttgtttttttatttgtatttttgtatggagttagttgagtttttattttttttttgttttggtttgttattagttgttggaatgtggaagattttgttttttttttttagggtggatttggagaaagatttgggaatagataggaaagaagttttgttttggattataagtatttaggagtattttatttataggaagggggaaagttagattataaaatgtttaaagaggtggaaaaagagatttaggttattaatttaggattgtaaggtgttttggaattttttaggtatttttattattggagaattgtgtgttagatgttattggtgtgattattaggtttagagaattaggtttaggtattaggaaaaagaaatagggattgtgaagtttagtatgtttggtagaaatggggtggaaatttttatttaagtaaagaaagtggagttgtgagtgatgttttagataaaattttataaaattttttataaaatgggtggtgtttagtatgttaaaattttagtttagagtttgggtgtaagggttgagttgagtgtagatttttgggtttgtttttatgttagttagttttgagttattttttattgtggaaaggtgggaaaattataagatattaattaattgaaaaggagggttagttatggaggtgtatatttgtaattttagttatttgggagggtgaggtagaaggattatttgaatttgggaggtagaggttgtagtgagttaagattgtgttattgtattttagtttgagtgatagagtgagattttgttttaaaaatagaaaaggaagttaagtatggtggtttatatttttaatgttaatgttttgggaggttaaggtaggtggattatttgtaattaggaatttgaggttagtttggttaatatggtgaaattttatttttattaaatatataaaaattagttgggtatggtggtgtgtgattgtagttttagttatttgggagattgaattattttaattgggaggtaaaggttgtagtgagttaagattgtgttattgtattttaatttgggtgatagggtgaggttttgttttaaaaaaaagaaagaaggttgggtttggtgatttatgtttgtaattttagtattttgggaggttaaggtaggtagattatttgaggttaagagtttgagatttgttaggttaatatagtaaaattttgtttgtattgaaaatataaaaaaattatttggttatggtggtgtgtgtttgtaattttagttattggggaggttgaggtaggagtattatttgaatttagaagatagaggttgtagtgagttgagattgggttattgtattttagtttggatgagagagtaagattttgttttaaaaaaaaaaaaaaaaaaaaagaaagaataggaggttgagaagttttaagttatatgttaaaaaaaaagaaaaaaatattagttttaggttaggtgtagtggtttatatttttaattttagtattttggaaagttgaggtgggtggattatgaggttaggagtttaagattagtttggttaaaatggtgaaattttgttttgattaaaaatataaaaaattagttagttgtggtggtaggtatttgtaattttagttatttgggaggttgaagtagagaattgtttgaatttaggaggtagagattgtaatgagttaagattgtattattgtattttagtttggaaaatagagtgagattttgttttaaaaaaaaaattattagtttttatggatagtggtagagtggagggtgggtttttatggtgtagaagggaaattttatggttttgttgtgtatttgattgggatggttgttgaaatttttttttagtaggtagttttggaaatagaaaaagaaatttttttttttttagaattttggaagggttgtgtagtgtttttaatttaagtttgttttttgagtgaagatagggaggtttattattagaagggaaggggttggaaatgaggttattgtattttagtttagggtttttgggttatttaggaagggaagaaggagtaagtttttttattgttaggtaggagtttagagttattataagaataagttagtattatttttgtgttttttttgttttgtaaataaaatgatttttttttttgttttggtattagagtttgtttggtattttttttgtttttagtattttttttatttgggtatttttttttgttggtgtattgaataaatatatttattgttttatttatagtttttagtttttattttttagggtttatattatttgtttttattaatttgataaggttgtttattgtttttagtaaggtttgtattggggtttttattttagtgtttttttttatttaggagatttttggatatttggggaagaaaatgagtttaaatttttatttttttttttttattttttttttgtaaggttttggttttagtttttagttttatatttttgttggttgtagaatagtagtgggttttgggtaaggagtattttgttaaaatgttttattttgtttttttatttgttttttttatttgtttttattagatggtttaagtgtttaaggggattttagggtggagttagggagaattttggtttttttgggttaggtataagattattttataggaaattttgtgggaatttttttgggataaagtattggttagtgttgagtttagttgtgtttgtgatatttgtattttaattagggtttatttgatgttaataggaagtaaggttgatgtagtggggttaagggagtttgggagaagaaagttggtttagagttttggttgttttgttttatattttatttttttggtaagaatttagtttttagatgaggtggggagtgagtggttgagttaaaaatttttgggttgggtatgatggtttatgtttgtaattttagtattttgggaggtgaaggtaggtggattatttgaggttaggagtttaagattaatttggttaatgtggtgaaattttatttttattaaaaatataaaaattagttgggtgttgttgtggtatgtgtttgtagttttagttatttgggagtttgaggtaggagaattgtttgaatttaggaggtagaatttgtagtgagttaagatttagttattgtattatagtttgggtgatagagtgaggttttgttttaaaaaaaaaaaaaatttttgggttaaatttttagatagtataggtaggtgtagaaatttattaggaagttgtttgtgtatttttggtagattggagtttggtttaaagttgttttttatgtagtttgggttaaggttaaatattatgttatagtgattttttttattatgtgtgagatatggagaattggttttaagtattattttgtttattggtggttggattattgatgtgtattattttttattttttttattttgtagtgggttatggttttgtgttggggtagaggagaaaaatgggttgttttttttaggataaatttttattttaatttaattagggtgttgtgattagaatgtgtaattgaggtgtgattttattgattttttttttttttgagattgagttttgtttttgttgtttaggttggagtgtgatggtatgattttagtttattgtaatttttattttttgagtttgagtaatttttttgttttagttttttaagtagttgggattataggtatgtgttattatgtttggttaattttgtatttttagtagagatggggtttttttatgttggttaggttggttttaaatttttgattttaggtgatttatttgttttggttttttaaagtgttagaattataggtgtgagttaatgtgtttagtttgtttttgttttttgtgttttgaagtagggttttatttagttttttaggttggagtgtagtgatatgataatagtttattgtagttgtaattttttgggtttaaatgatttttttattttagttttttgaatagttgggattataggtatattattatatttggttaatttttttttttttttttttagtagagatgaggttttgttatgttgtttaagttggttttaaatttttgaggattaagtgatttttttattttagttttttaaaatgttgggattgtagatgtgagttattatatttagtttgattttattttaaatgagagtttttttttagagttttttagttgtttttggtttttggttatgtgtttttagttgtttttgtttttgtggtatttttaaggttatatttagtgttgaggttttaggtaggtagtagagagaagttaaatgattttgttttttttttatttatttagagtatgtaaaattaggagtagtggtgggtttagggtgggtattagttatgtatatgtatattagggatagggggttaaaggtagttagtttttaaagattgttttagaggttattttttagagaagttttgggttttttaagggttttgtgtttatgttggtttattttgtaggatgagtttgtggagtgggagatatttgattttttttaagttgagattgagtagaagattaaggagtataatgtttagattaatagtaatttttttatgagtttggtgagttgattgtttaggaagggggtgtggggaggagtaggtatttagttatgtgtttgatatttagagggttataattgaggttattttgggtgggtgtaagtagtaatttgtgtatatttagtttagttttaagtagattgatattttatttggaatttattattaaggtttggtttttttatttttttagaataaggatggtttttatataggttttattaaggtttagttgaagttggtgtgttttgtttttgtgttttttagtaagaagttattttttttgtaggatgtttggtggggtttaggatggggtataagtgttaggtgttgtatttttttttatttgtttaaggatgttgttaagtatttgtatgtgttgttatgtataagggtatgtgaagttattgaggttttgttgtgaaagtttttggtggtggatgatttttgtaagtttgtattttttgagtgtgttgagtgttatggttaaggtgggttttttattttattttgttttatgtgagggtatatatgtatgtatttgagtatgtaggggttgagtagttggttttgtttttgattattattttttttttatagtgtatttgtggaagttgttggatgatgagtagtttttgtggttgtggtttttggtagggtttagtgataaggttttgagttttgttttgaaggaaaatgattttggggaggtgaatgtgagtatatagtttttagttttttggttgttattagataggattgatgggttgtagttatagtaaggtttggaggaggaattgtgttggaagataagttttgtaaaatagttttaggagtgtataggtattgtaattaaagtaaaggtttttagattatttatgttaaagtttagggttgttttaagaagttaggaagaattgttttggtgttttgattttttttggtgtggaaaattttttggagatgtaggagtttatttaatgatatgaggaggttttttttagattttttatttggaagttttttggttttaaggtattaggtttgtggagtgaaattagatttagaatatgtttgatttgtttataggtaattggggaatatttgatttgg

SEQ ID NO: 26 - DNA, 25 bp, artificial sequence
  Primer
tggtgatggaggaggtttagtaagt

SEQ ID NO: 27 - DNA, 27 bp, artificial sequence
  Primer
aaccaataaaacctactcctcccttaa

SEQ ID NO: 28 - DNA, 30 bp, artificial sequence
  Probe
accaccacccaacacacaataacaaacaca

SEQ ID NO: 29 - DNA, 20 bp, artificial sequence
  Primer
attgagttgcgggagttggt

SEQ ID NO: 30 - DNA, 20 bp, artificial sequence
  Primer
acacgctccaaccgaatacg

SEQ ID NO: 31 - DNA, 18 bp, artificial sequence
  Probe
cccttcccaacgcgccca

(19) United States
(12) Patent Application Publication
(10) Pub. No.: US 2009/0162836 A1
(43) Pub. Date: Jun. 25, 2009

(54) Prognostic and Diagnostic Markers for Cell Proliferative Disorders of The Breast Tissues

(75) Inventor: Martin Widschwendter, Tonbridge (GB)

Correspondence Address: DAVIS WRIGHT TREMAINE, LLP/Seattle, 1201 Third Avenue, Suite 2200, SEATTLE, WA, 98101-3045, US

(73) Assignee: Epigenomics AG, Berlin (DE)

(21) Appl. No.: 10/575,753

(22) PCT Filed: Oct. 14, 2004
(86) PCT No.: PCT/EP04/11577

§ 371 Date: Jan. 3, 2007

(30) Foreign Application Priority Data
Oct. 17, 2003 (DE) 103 48 407.8

(51) Int. Cl. C12Q 1/68; C07H 21/04

(52) U.S. Cl. 435/6; 536/23.1; 536/25.3

(57) ABSTRACT

The present invention relates to prognostic and diagnostic markers for cell proliferative disorders of the breast tissues. The present invention therefore provides methods and nucleic acids for the analysis of biological samples for features associated with the development of breast cell proliferative disorders. Furthermore, the invention provides for prognosis of treatment effects relating to drug therapy, in particular hormonal/antihormonal therapy, chemotherapy and/or adjuvant therapy.

The present invention relates to prognostic and diagnostic markers for cell proliferative disorders of the breast tissues. The present invention therefore provides methods and nucleic acids for the analysis of biological samples for features associated with the development of breast cell proliferative disorders. Furthermore, the invention provides for prognosis of treatment effects relating to drug therapy, in particular hormonal/antihormonal therapy, chemotherapy and/or adjuvant therapy.

Accordingly, this invention relates to the diagnosis and prognosis of cell proliferative disorders, in particular breast cancer, and the prognosis of a treatment regime success in cell proliferative disorders of breast tissues.

Today involvement of axillary lymph nodes and tumour size are the most important prognostic factors in breast cancer. Although the presence or absence of metastatic involvement in the axillary lymph nodes is the most powerful prognostic factor available for patients with primary breast cancer, it is only an indirect measure reflecting the tumours' tendency to spread. In approximately one-third of women with breast cancer and negative lymph nodes the disease recurs, while about one-third of patients with positive lymph nodes are free of recurrence ten years after loco-regional therapy. These data highlight the need for more sensitive and specific prognostic indicators, ideally reflecting the presence or absence of tumour-specific alterations in the bloodstream that may eventually even after years lead to metastasis. It is now widely accepted that adjuvant systemic therapy substantially improves disease-free and overall survival in both pre- and postmenopausal women up to the age of 70 years with lymph node-negative or lymph node-positive breast cancer (early Breast Cancer Trialists' Collaborative Group Tamoxifen for early breast cancer: an overview of the randomised trials. Early Breast Cancer Trialists' Collaborative Group. Lancet, 351: 1451-1467, 1998.2, 3). It is also generally accepted that patients with poor prognostic features benefit the most from adjuvant therapy, whereas some patients with good prognostic features may be overtreated (Goldhirsch et al.: Meeting highlights: International Consensus Panel on the Treatment of Primary Breast Cancer. Seventh International Conference on Adjuvant Therapy of Primary Breast Cancer. J. Clin. Oncol., 19: 3817-3827, 2001.). Moreover many other factors have been investigated for their potential to predict disease outcome, but in general they have only limited predictive value. Recently, interesting prognostic parameters including gene-expression profiles, cell cycle regulating proteins and occult cytokeratin-positive metastatic cells in the bone marrow have been added to the list of prognostic factors, but their prognostic relevance needs to be further evaluated.

Changes in the status of DNA methylation, known as epigenetic alterations, are one of the most common molecular alterations in human neoplasia, including breast cancer (Widschwendter and Jones: DNA methylation and breast carcinogenesis. Oncogene, 21: 5462-5482, 2002). Cytosine methylation occurs after DNA synthesis by enzymatic transfer of a methyl group from the methyl donor S-adenosylmethionine to the carbon-5 position of cytosine. Cytosines are methylated in the human genome mostly when located 5′ to a guanosine. Regions with a high G:C content are so-called CpG islands. It has been increasingly recognized over the past four to five years that the CpG islands of a large number of genes, which are mostly umethylated in normal tissue, are methylated to varying degrees in human cancers, thus representing tumor-specific alterations. The presence of abnormally high DNA concentrations in the serum of patients with various malignant diseases was described several years ago. The discovery that cell-free DNA can be shed into the bloodstream has generated great interest. Numerous studies have demonstrated tumor-specific alterations in DNA recovered from plasma or serum of patients with various malignancies, a finding that has potential for molecular diagnosis and prognosis. The nucleic acid markers described in plasma and serum include oncogene mutations, microsatellite alterations, gene rearrangements and epigenetic alterations, such as aberrant promoter hypermethylation (Anker et al.: Detection of circulating tumour DNA in the blood (plasma/serum) of cancer patients. Cancer Metastasis Rev., 18: 65-73, 1999). During recent years some studies have reported cell-free DNA in serum/plasma of breast cancer patients at diagnosis (for example: Silva et al.: Presence of tumor DNA in plasma of breast cancer patients: clinicopathological correlations. Cancer Res., 59: 3251-3256, 1999) and in some cases persistence after primary therapy (for example: Silva et al.: Persistence of tumor DNA in plasma of breast cancer patients after mastectomy. Ann. Surg. Oncol., 9: 71-76, 2002). Nevertheless an increasing number of studies have reported the presence of methylated DNA in serum/plasma of patients with various types of malignancies, including breast cancer, and the absence of methylated DNA in normal control patients (for example: Wong et al.: Detection of aberrant p16 methylation in the plasma and serum of liver cancer patients. Cancer Res., 59: 71-73, 1999). So far, only few studies have addressed the prognostic value of these epigenetic alterations in patients' bloodstream (Kawakami et al.: Hypermethylated APC DNA in plasma and prognosis of patients with esophageal adenocarcinoma. J. Natl. Cancer Inst., 92: 1805-1811, 2000; Lecomte et al.: Detection of free-circulating tumor-associated DNA in plasma of colorectal cancer patients and its association with prognosis. Int. J. Cancer, 100: 542-548, 2002).

It will be appreciated by those skilled in the art that there exists a continuing need to improve methods of early detection, classification and treatment of breast cancers. In this application prognostic and diagnostic DNA methylation-based markers for breast cancer are disclosed.

5-methylcytosine positions cannot be identified by sequencing since 5-methylcytosine has the same base pairing behavior as cytosine. Moreover, the epigenetic information carried by 5-methylcytosine is completely lost during PCR amplification. Currently the most frequently used method for analysing DNA for 5-methylcytosine is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its hybridisation behaviour, can now be detected as the only remaining cytosine using “normal” molecular biological techniques, for example, by amplification and hybridisation or sequencing. All of these techniques are based on base pairing which can now be fully exploited. In terms of sensitivity, the prior art is defined by a method which encloses the DNA to be analysed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. 1996 December 15;24(24):5064-6). Using this method, it is possible to analyse individual cells, which illustrates the potential of the method. However, currently only individual regions of a length of up to approximately 3000 base pairs are analysed, a global analysis of cells for thousands of possible methylation events is not possible. However, this method cannot reliably analyse very small fragments from small sample quantities either. These are lost through the matrix in spite of the diffusion protection.

An overview of the further known methods of detecting 5-methylcytosine may be gathered from the following review article: Fraga and Esteller: DNA Methylation: A Profile of Methods and Applications. Biotechniques 33:632-649, September 2002.

To date, barring few exceptions (e.g., Zeschnigk M, Lich C, Buiting K, Doerfier W, Horsthemke B. A single-tube PCR test for the diagnosis of Angelman and Prader-Willi syndrome based on allelic methylation differences at the SNRPN locus. Eur J Hum Genet. 1997 March-April; 5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat. Genet. 1997 November; 17(3):275-6) or individual cytosine positions are detected by a primer extension reaction (Gonzalgo M L, Jones P A. Rapid quantitation of methylation differences at specific sites using methylation-sensitive single nucleotide primer extension (Ms-SNuPE). Nucleic Acids Res. 1997 Jun. 15; 25(12):2529-31, WO 95/00669) or by enzymatic digestion (Xiong Z, Laird P W. COBRA: a sensitive and quantitative DNA methylation assay. Nucleic Acids Res. 1997 Jun. 15; 25(12):2532-4). In addition, detection by hybridisation has also been described (Olek et al., WO 99/28498).

Further publications dealing with the use of the bisulfite technique for methylation detection in individual genes are: Grigg G, Clark S. Sequencing 5-methylcytosine residues in genomic DNA. Bioessays. 1994 June; 16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. 1997 March; 6(3):387-95; Feili R, Charlton J, Bird A P, Walter J, Reik W. Methylation analysis on individual chromosomes: improved protocol for bisulphite genomic sequencing. Nucleic Acids Res. 1994 Feb. 25; 22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. 1995 May 19; 157(1-2):261-4; WO 97/46705, WO 95/15373, and WO 97/45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labelled probes are often used for the scanning of immobilised DNA arrays. The simple attachment of Cy3 and Cy5 dyes to the 5′-OH of the specific probe are particularly suitable for fluorescence labels. The detection of the fluorescence of the hybridised probes may be carried out, for example via a confocal microscope. Cy3 and Cy5 dyes, besides many others, are commercially available.

Matrix Assisted Laser Desorption Ionisation Mass Spectrometry (MALDI-TOF) is a very efficient development for the analysis of biomolecules (Karas M, Hillenkamp F. Laser desorption ionisation of proteins with molecular masses exceeding 10,000 daltons. Anal Chem. 1988 Oct. 15; 60(20):2299-301). An analyte is embedded in a light-absorbing matrix. The matrix is evaporated by a short laser pulse thus transporting the analyte molecule into the vapour phase in an unfragmented manner. The analyte is ionised by collisions with matrix molecules. An applied voltage accelerates the ions into a field-free flight tube. Due to their different masses, the ions are accelerated at different rates. Smaller ions reach the detector sooner than bigger ones.

MALDI-TOF spectrometry is excellently suited to the analysis of peptides and proteins. The analysis of nucleic acids is somewhat more difficult (Gut I G, Beck S. DNA and Matrix Assisted Laser Desorption Ionisation Mass Spectrometry. Current Innovations and Future Trends. 1995, 1; 147-57). The sensitivity to nucleic acids is approximately 100 times worse than to peptides and decreases disproportionally with increasing fragment size. For nucleic acids having a multiply negatively charged backbone, the ionisation process via the matrix is considerably less efficient. In MALDI-TOF spectrometry, the selection of the matrix plays an eminently important role. For the desorption of peptides, several very efficient matrixes have been found which produce a very fine crystallisation. There are now several responsive matrixes for DNA, however, the difference in sensitivity has not been reduced. The difference in sensitivity can be reduced by chemically modifying the DNA in such a manner that it becomes more similar to a peptide. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. 1995 Apr. 25; 23(8):1367-73). The coupling of a charge tag to this modified DNA results in an increase in sensitivity to the same level as that found for peptides. A further advantage of charge tagging is the increased stability of the analysis against impurities which make the detection of unmodified substrates considerably more difficult.

Genomic DNA is obtained from DNA of cell, tissue or other test samples using standard methods. This standard methodology is found in references such as Fritsch and Maniatis eds., Molecular Cloning: A Laboratory Manual, 1989.

The present invention provides methods and nucleic acids for the analysis of biological samples for features associated with the development of breast cell proliferative disorders and/or for the prognosis of treatment regimes in the medical intervention of breast cell proliferative disorders. The invention is characterised in that the nucleic acid of at least one member of the group of genes according to Table 1 (or a fragment of said genes) is/are contacted with a reagent or series of reagents capable of distinguishing between methylated and non methylated CpG dinucleotides within the genomic sequence (or within a part of said genomic sequence) of interest. The present invention makes available a method for ascertaining genetic and/or epigenetic parameters of genomic DNA. The method is for use for the determining the prognosis of breast cell proliferative disorders. The invention presents improvements over the state of the art in that by means of the methods and compounds described herein a person skilled in the art may carry out a sensitive and specific detection assay of cellular matter comprising cancerous breast tissue. This is particularly useful as it allows the analysis of samples of body fluids which may contain only a minimal amount of cell proliferative disorder cellular matter, and enables the detection of said cells and the identification of the organ from which they originated (in this case breast). To date there are no known clinically utilisable means for the detection of breast cancer using genetic methylation markers to analyse bodily fluid samples, such as blood, lymphatic fluids, nipple aspirate and plasma. The generated information is useful in the selection of a treatment of the patient. If a positive prognosis is determined a further treatment might be redundant, while in a case of a poor prognosis a stronger treatment might be necessary. Furthermore, the invention provides for means and methods for the evaluation whether treatment and/or intervention regimes in breast cell proliferative disorder management are fruitful. In this context and in a preferred embodiment the treatment success and/or potential treatment success of hormonal/antihormonal therapy (in particular tamoxifen therapy) is envisaged.

Furthermore, the method enables the analysis of cytosine methylations and single nucleotide polymorphisms.

The genes that form the basis of the present invention are preferably to be used to form a “gene panel”, i.e. a collection comprising the particular genetic sequences of the present invention and/or their respective informative methylation sites. The formation of gene panels allows for a quick and specific analysis of specific aspects of breast cancer. The gene panel(s) as described and employed in this invention can be used with surprisingly high efficiency for the diagnosis, treatment and monitoring of and the analysis of a predisposition to breast cell proliferative disorders.

In addition, the use of multiple CpG sites from a diverse array of genes allows for a relatively high degree of sensitivity and specificity in comparison to single gene diagnostic and detection tools. Of the genes known to be specifically methylated in breast cancer, the particular combination of the genes according to the invention provides for a particularly sensitive and specific means for the identification of cell proliferative disorders of breast tissues.

The object of the invention is most preferably achieved by means of the analysis of the methylation patterns of one or a combination of genes taken from the group taken from the group ESR1, APC, HSD174B4, HIC1 and RASSF1A (see, for example, Table 1) and/or their regulatory regions. The corresponding genes as well as their regulatory sequences are known in the art and e.g. defined by this genomic sequences as given in Table 1 and in particular in SEQ ID NOS: 1 to 5. The methylation pattern of these genes may also be deduced from fragments of the corresponding genes and/or their regulatory sequences as well as from fragments of their corresponding complementary strand. Such fragments comprise correspondingly CpG dinucleotides and comprise preferably at least 10 nucleotides, more preferably, at least 20 nucleotides, more preferably at least 50 nucleotides and most preferably at least 100 nucleotides. As demonstrated in the appended examples, fragments between 50 and 150 nucleotides may be used, inter alia in MethyLight® technology. Primers and probes to be employed (e.g. in MethyLight) comprise between preferably between 9 and 20, most preferably 14 nucleotides.

The invention is characterised in that the nucleic acid of one or a combination of genes taken from the group ESR1, APC, HSD174B4, HIC1 and RASSF1A are contacted with a reagent or series of reagents capable of distinguishing between methylated and non methylated CpG dinucleotides within the genomic sequence of interest.

The object of the invention can also be achieved by the analysis of the CpG methylation of one or a plurality of any subset of the group of genes ESR1, APC, HSD174B4, HIC1 and RASSF1A, in particular the following subsets are preferred:
RASSF1A and APC,
RASSF1A, and
APC

Accordingly, in a most preferred embodiment, the CpG methylation of RASSF1A is investigated in accordance with this invention and in particular in the context of selecting a suitable treatment regime (in accordance with the prognosis of the patient). Most preferably, said treatment regime is a tamoxifen treatment.

As documented in the appended examples, in particular RASSF1A DNA methylation is also a particularly useful, prognostic marker in patients with breast cancer metastasis. This is in particular useful in predictions of survival rates in metastatic breast cancer.

The present invention makes available a method for ascertaining genetic and/or epigenetic parameters of genomic DNA. The method is, accordingly, for use in the improved diagnosis, treatment and monitoring of breast cell proliferative disorders. The disclosed invention further provides a method for determining the phenotype of a subject with a breast cell proliferative disorder comprising

a) obtaining a biological sample containing genomic DNA from said subject,
b) analysing the methylation pattern of one or more target nucleic acids comprising one or a combination of the genes taken from the group consisting of ESR1, APC, HSD174B4, HIC1 and RASSF1A and/or their regulatory regions by contacting at least one of said target nucleic acids in the biological sample obtained from said subject with at least one reagent, or series of reagents that distinguishes between methylated and non-methylated CpG dinucleotides, and
c) determining the phenotype of the individual by comparison to two known phenotypes, a first phenotype characterised by hypermethylation of the target nucleic acid and poor prognosis as relative to a second phenotype characterised by hypomethylation of the analysed target nucleic acid and better prognosis

The corresponding “target nucleic acids” comprise but are not limited to the nucleic acid molecules provided in Table 1 and the corresponding SEQ ID NOS 1 to 5. The term, however, also comprises target sequences which are homologous or at least 80%, more preferably at least 85%, more preferably at least 90%, more preferably at least 95% and most preferably at least 99% identical to the nucleic acid sequences as provided in the SEQ ID NOS: 1 to 5. Accordingly, the genes taken from the group consisting of ESR1, APC, HSD174B4, HIC1 and RASSF1A are not limited to the genes as shown in SEQ ID NOS: 1 to 5 but said form also comprises variants of said sequences, like allelic variants, in particular naturally occurring variants. The term “genes taken or selected from the group consisting of ESR1, APC, HSD174B4, HIC1 and RASSF1A also comprises sequences which hybridize, preferably under stringent conditions, to the complementary strand of the sequences as shown in SEQ ID NOS: 1 to 5.

In context of the present invention, the term “identity” or “homology” as used herein relates to a comparison of nucleic acid molecules (nucleotide stretches; DNA, RNA). Accordingly, also a variant of the genes selected from the group consisting of ESR1, APC, HSD174B4, HIC1 and RASSF1A may be determined by sequence comparison.

In order to determine whether a nucleic acid sequence has a certain degree of identity to the nucleic acid sequence encoding ESR1, APC, HSD174B4, HIC1 and RASSF1A the skilled person can use means and methods well-known in the art, e.g., alignments, either manually or by using computer programs such as those mentioned further down below in connection with the definition of the term “hybridization” and degrees of homology.

For example, BLAST2.0, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403410), can be used to search for local sequence alignments. BLAST produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules.

The present invention also relates to use of ESR1, APC, HSD174B4, HIC1 and RASSF1A-mutants comprising mutations in nucleic acid molecules which hybridize to one of the above described nucleic acid molecules represented in SEQ ID NOS: 1 to 5.

The term “hybridizes” as used in accordance with the present invention may relate to hybridization under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which represent a ESR1, APC, HSD174B4, HIC1 and RASSF1A gene as defined herein and which have a length of at least 12 nucleotides, preferably at least 15, more preferably at least 18, more preferably of at least 21 nucleotides, more preferably at least 30 nucleotides, even more preferably at least 40 nucleotides and most preferably at least 60 nucleotides. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 80%, preferably at least 85%, more preferably at least 90%, even more preferably at least 95%, even more particularly preferred at least 96%, 97% or 98% and most preferably at least 99% identity with a nucleic acid sequence as described in SEQ ID NOS: 1, 2, 3, 4 or 5. In accordance with the present invention, the term “identical” or “percent identity” in the context of two or more nucleic acid sequences, refers to two or more sequences or subsequences that are the same, or that have a specified percentage of amino acid residues or nucleotides that are the same (e.g., 70-95% identity, more preferably at least 95%, 97%, 98% or 99% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 5 to 30 amino acids or nucleotides in length, more preferably, over a region that is about 5 to 30 amino acids or nucleotides in length. Those having skill in the art will know how to determine percent identity between/among sequences using, for example, algorithms such as those based on CLUSTALW computer program (Thompson, Nucl. Acids Res. 2 (1994), 4673-4680) or FASTDB (Brutlag, Comp. App. Biosci. 6 (1990), 237-245), as known in the art.

The above recited method is preferably carried out by analysing the methylation pattern of RASSF1A and/or its regulatory sequences/regions when the prognosis of survival rates in metastatic breast cancer is to be determined or when the treatment success or treatment prognosis, e.g. of a tamoxifen treatment is to be determined.

The DNA may be obtained from any form of biological sample including but not limited to cell lines, histological slides, biopsies, tissue embedded in paraffin, breast tissues, blood, plasma, lymphatic fluid, lymphatic tissue, duct cells, ductal lavage fluid, nipple aspiration fluid and combinations thereof. Genomic DNA must then be isolated from the sample using any means standard in the art. The isolated DNA is treated with at least one reagent, or series of reagents that distinguishes between methylated and non-methylated CpG dinucleotides. This may be carried out by any means standard in the art including the use of restriction endonucleases. However, it is preferably carried out with bisulfite (sulfite, disulfite) and subsequent alkaline hydrolysis which results in a conversion of non-methylated cytosine nucleobases to uracil or to another base which is dissimilar to cytosine in terms of base pairing behaviour. If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The converted DNA is then used for the detection of methylated cytosines. The methylation status of one or more of the genes ESR1, APC, HSD174B4, HIC1 and RASSF1A and/or of their regulatory regions (or of fragments of said genes and/or of fragments of said regulatory sequences) is then analysed. This analysis may be carried out by any means standard in the art including the above described techniques. In the final step of the method the methylation pattern of the DNA obtained from the subject is compared to that of two known phenotypes. The first phenotype is characterised by hypermethylation or methylation of the target nucleic acid and poor prognosis as relative to a second phenotype characterised by hypomethylation or no methylation of the analysed target nucleic acid and better prognosis. For example, appended Table 3 provides for results of a diagnostic analysis of prognosis employing the methylation status of the genes and/or their regulatory sequences provided herein above. It is particularly preferred that the genes APC and/or RASSF1A are analysed. Most preferably, the methylation status of RASSF1A is analyzed. By determining which of the two phenotypes the subject belongs to it is possible to determine a suitable treatment to her breast cell proliferative disorder. Also the treatment success, for example in a hormonal/antihormonal therapy may be determined as shown in the appended examples.

The method according to the invention may be used for the analysis of a wide variety of cell proliferative disorders of the breast tissues including, but not limited to, ductal carcinoma in situ, lobular carcinoma, colloid carcinoma, tubular carcinoma, medullary carcinoma, metaplastic carcinoma, intraductal carcinoma in situ, lobular carcinoma in situ and papillary carcinoma in situ.

Furthermore, the method enables the analysis of cytosine methylations and single nucleotide polymorphisms within said genes.

The object of the invention is achieved by means of the analysis of the methylation patterns of one or more of the genes ESR1, APC, HSD174B4, HIC1 and RASSF1A and/or their regulatory regions. As mentioned above, in a particularly preferred embodiment the sequences of said genes comprise SEQ ID NOs: 1 to 5 and sequences complementary thereto. As discussed above, in a most preferred embodiment, for example in the determination of a treatment success or a potential treatment success with, e.g. tamoxifen, the RASSF1A gene methylation pattern is analysed. A specific example is given in the experimental part.

The object of the invention may also be achieved by analysing the methylation patterns of one or more genes (or fragments of said genes) taken from the following subsets of said aforementioned group of genes. In one embodiment the object of the invention is preferably achieved by analysis of the methylation patterns of the genes RASSF1A and APC and wherein it is further preferred that the sequence of said genes comprise SEQ ID NOs: 5 and 3, respectively. In a further embodiment the object of the invention is achieved by analysis of the methylation patterns of the gene RASSF1A and/or its regulatory sequences, and wherein it is further preferred that the sequence of said gene comprises or is SEQ ID NO: 5. In further aspects, the object of the invention may also be achieved by analysis of the methylation pattern of the gene APC and/or its regulatory sequences, and wherein it is further preferred that the sequence of said gene comprises or is SEQ ID NO: 3. as mentioned above also (highly) homologous sequences which are at least 80% identical to the sequences as shown in SEQ ID NO: 5 (RASSF1A) or SEQ ID NO: 3 (APC).

In a preferred embodiment said method is achieved by contacting said nucleic acid sequences in a biological sample obtained from a subject with at least one reagent or a series of reagents, wherein said reagent or series of reagents, distinguishes between methylated and non methylated CpG dinucleotides within the target nucleic acid.

In a preferred embodiment, the method comprises the following steps:

In the first step of the method the genomic DNA sample must be isolated from sources such as cells or cellular components which contain DNA, sources of DNA comprising, for example, cell lines, histological slides, biopsies, tissue embedded in paraffin, breast tissues, blood, plasma, lymphatic fluid, lymphatic tissue, duct cells, ductal lavage fluid, nipple aspiration fluid and combinations thereof. Extraction may be by means that are standard to one skilled in the art, these include the use of detergent lysates, sonification and vortexing with glass beads. Once the nucleic acids have been extracted the genomic double stranded DNA is used in the analysis.

Details to the methods of the present invention are given in the appended examples.

In one embodiment the DNA may be cleaved prior to the next step of the method, this may be by any means standard in the state of the art, in particular, but not limited to, with restriction endonucleases.

In the second step of the method, the genomic DNA sample is treated in such a manner that cytosine bases which are unmethylated at the 5′-position are converted to uracil, thymine, or another base which is dissimilar to cytosine in terms of hybridisation behaviour. This will be understood as “pretreatment” or “chemical pretreatment” hereinafter.

The above described treatment of genomic DNA is preferably carried out with bisulfite (sulfite, disulfite) and subsequent alkaline hydrolysis which results in a conversion of non-methylated cytosine nucleobases to uracil or to another base which is dissimilar to cytosine in terms of base pairing behaviour. If bisulfite solution is used for the reaction, then an addition takes place at the non-methylated cytosine bases. Moreover, a denaturating reagent or solvent as well as a radical interceptor must be present. A subsequent alkaline hydrolysis then gives rise to the conversion of non-methylated cytosine nucleobases to uracil. The converted DNA is then used for the detection of methylated cytosines.

Fragments (e.g. fragments comprising preferably about 100 bp or most preferably at least 90 bp) of the pretreated DNA are amplified, using sets of primer oligonucleotides, and a preferably heat-stable, polymerase. Because of statistical and practical considerations, preferably more than six different fragments having a length of 100-2000 base pairs (bp) are amplified. However, fragments of at least 50 bp may be amplified. The amplification of several DNA segments can be carried out simultaneously in one and the same reaction vessel. Usually, the amplification is carried out by means of a polymerase chain reaction (PCR).

The design of such primers is known to one skilled in the art. These should include at least two oligonucleotides whose sequences are each reverse complementary or identical to an at least 18 base-pair long segment of the following base sequences specified in the appendix: SEQ ID NO 6 to 26. Said primer oligonucleotides are preferably characterised in that they do not contain any CpG dinucleotides. In a particularly preferred embodiment of the method, the sequence of said primer oligonucleotides are designed so as to selectively anneal to and amplify, only the breast cell specific DNA of interest, thereby minimising the amplification of background or non relevant DNA. In the context of the present invention, background DNA is taken to mean genomic DNA which does not have a relevant tissue specific methylation pattern, in this case, the relevant tissue being breast tissues.

According to the present invention, it is preferred that at least one primer oligonucleotide is bound to a solid phase during amplification. The different oligonucleotide and/or PNA-oligomer sequences can be arranged on a plane solid phase in the form of a rectangular or hexagonal lattice, the solid phase surface preferably being composed of silicon, glass, polystyrene, aluminium, steel, iron, copper, nickel, silver, or gold, it being possible for other materials such as nitrocellulose or plastics to be used as well.

The fragments obtained by means of the amplification may carry a directly or indirectly detectable label. Preferred are labels in the form of fluorescence labels, radionuclides, or detachable molecule fragments having a typical mass which can be detected in a mass spectrometer, it being preferred that the fragments that are produced have a single positive or negative net charge for better detectability in the mass spectrometer. The detection may be carried out and visualised by means of matrix assisted laser desorption/ionisation mass spectrometry (MALDI) or using electron spray mass spectrometry (ESI).

In the next step the nucleic acid amplificates are analysed in order to determine the methylation status of the genomic DNA prior to treatment.

The post treatment analysis of the nucleic acids may be carried out using alternative methods. Several methods for the methylation status specific analysis of the treated nucleic acids are described below, other alternative methods will be obvious to one skilled in the art.

The analysis may be carried out during the amplification step of the method. In one such embodiment, the methylation status of preselected CpG positions within the genes ESR1, APC, HSD174B4, HIC1 and RASSF1A and/or their regulatory regions may be detected by use of methylation specific primer oligonucleotides. The term “MSP” (Methylation-specific PCR) refers to the art-recognized methylation assay described by Herman et al. Proc. Natl. Acad. Sci. USA 93:9821-9826, 1996, and also disclosed in U.S. Pat. No. 5,786,146 and No. 6,265,171. The use of methylation status specific primers for the amplification of bisulphite treated DNA allows the differentiation between methylated and unmethylated nucleic acids. MSP primers pairs contain at least one primer which hybridises to a bisulphite treated CpG dinucleotide. Therefore the sequence of said primers comprises at least one CG, TG or CA dinucleotide. MSP primers specific for non methylated DNA contain a ‘T’ at the 3′ position of the C position in the CpG. According to the present invention, it is therefore preferred that the base sequence of said primers is required to comprise a sequence having a length of at least 10 nucleotides which hybridises to a pretreated nucleic acid sequence according to SEQ ID NOs.: 6 to 26 and sequences complementary thereto wherein the base sequence of said oligomers comprises at least one CG, TG or CA dinucleotide.

In one embodiment of the method the methylation status of the CpG positions may be determined by means of hybridisation analysis. In this embodiment of the method the amplificates obtained in the second step of the method are hybridised to an array or a set of oligonucleotides and/or PNA probes. In this context, the hybridisation takes place in the manner described as follows. The set of probes used during the hybridisation is preferably composed of at least 4 oligonucleotides or PNA-oligomers. In the process, the amplificates serve as probes which hybridise to oligonucleotides previously bonded to a solid phase. The non-hybridised fragments are subsequently removed. Said oligonucleotides contain at least one base sequence having a length of 10 nucleotides which is reverse complementary or identical to a segment of the base sequences specified in the appendix, the segment containing at least one CpG or TpG dinucleotide. In a further preferred embodiment the cytosine of the CpG dinucleotide, or in the case of TpG, the thiamine, is the 5^{th} to 9^{th} nucleotide from the 5′-end of the 10-mer. One oligonucleotide exists for each CpG or TpG dinucleotide.

The non-hybridised amplificates are then removed. In the final step of the method, the hybridised amplificates are detected. In this context, it is preferred that labels attached to the amplificates are identifiable at each position of the solid phase at which an oligonucleotide sequence is located.

In a preferred embodiment of the method the methylation status of the CpG positions may be determined by means of oligonucleotide probes that are hybridised to the treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996) employing a dual-labelled fluorescent oligonucleotide probe (TaqMan™ PCF, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which is designed to hybridise to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulphite treatment it is required that the probe be methylation specific, as described in U.S. Pat. No. 6,331,393, also known as the Methyl Light assay. Variations on the TaqMan™ detection methodology that are also suitable for use with the described invention include the use of dual probe technology (Lightcycler™) or fluorescent amplification primers (Sunrise™ technology). Both these techniques may be adapted in a manner suitable for use with bisulphite treated DNA, and moreover for methylation analysis within CpG dinucleotides.

A further suitable method for the use of probe oligonucleotides for the assessment of methylation by analysis of bisulphite treated nucleic acids is the use of blocker oligonucleotides. The use of such oligonucleotides has been described by D. Yu, M. Mukai, Q. Liu, C. Steinman in BioTechniques 23(4), 1997, 714-720. Blocking probe oligonucleotides are hybridised to the bisulphite treated nucleic acid concurrently with the PCR primers. PCR amplification of the nucleic acid is terminated at the 5′ position of the blocking probe, thereby amplification of a nucleic acid is suppressed wherein the complementary sequence to the blocking probe is present. The probes may be designed to hybridise to the bisulphite treated nucleic acid in a methylation status specific manner. For example, for detection of methylated nucleic acids within a population of unmethylated nucleic acids suppression of the amplification of nucleic acids which are unmethylated at the position in question would be carried out by the use of blocking probes comprising a ‘CG’ at the position in question, as opposed to a ‘CA’.

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivatised at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase.

Preferably, therefore, the base sequence of said blocking oligonucleotides is required to comprise a sequence having a length of at least 9 nucleotides which hybridises to a pretreated nucleic acid sequence according to one of SEQ ID NOs: 6 to 26 and sequences complementary thereto, wherein the base sequence of said oligonucleotides comprises at least one CpG, TpG or CpA dinucleotide.

In a further preferred embodiment of the method the determination of the methylation status of the CpG positions is carried out by the use of template directed oligonucleotide extension, such as MS SNuPE as described by Gonzalgo and Jones (Nucleic Acids Res. 25:2529-2531).

In a further embodiment of the method the determination of the methylation status of the CpG positions is enabled by sequencing and subsequent sequence analysis of the amplificate generated in the second step of the method (Sanger F., et al., 1977 PNAS USA 74: 5463-5467).

The method according to the invention may be enabled by any combination of the above means. In a particularly preferred mode of the invention the use of real time detection probes is concurrently combined with MSP and/or blocker oligonucleotides.

A further embodiment of the invention is a method for the analysis of the methylation status of genomic DNA without the need for pretreatment. In the first and second steps of the method the genomic DNA sample must be obtained and isolated from tissue or cellular sources. Such sources may include cell lines, histological slides, biopsies, tissue embedded in paraffin, breast tissues, blood, plasma, lymphatic fluid, lymphatic tissue, duct cells, ductal lavage fluid, nipple aspiration fluid and combinations thereof. Extraction may be by means that are standard to one skilled in the art, these include the use of detergent lysates, sonification and vortexing with glass beads. Once the nucleic acids have been extracted the genomic double stranded DNA is used in the analysis.

In a preferred embodiment the DNA may be cleaved prior to the treatment, this may be by any means standard in the state of the art, in particular with restriction endonucleases. In the third step, the DNA is then digested with one or more methylation sensitive restriction enzymes. The digestion is carried out such that hydrolysis of the DNA at the restriction site is informative of the methylation status of a specific CpG dinucleotide.

In a preferred embodiment the restriction fragments are amplified. In a further preferred embodiment this is carried out using the polymerase chain reaction.

In the final step the amplificates are detected. The detection may be by any means standard in the art, for example, but not limited to, gel electrophoresis analysis, hybridisation analysis, incorporation of detectable tags within the PCR products, DNA array analysis, MALDI or ESI analysis.

The aforementioned method is preferably used for ascertaining genetic and/or epigenetic parameters of genomic DNA.

In order to further enable this method, the invention further provides the modified DNA of one or a combination of genes taken from the group ESR1, APC, HSD174B4, HIC1 and RASSF1A as well as oligonucleotides and/or PNA-oligomers for detecting cytosine methylations within said genes. The present invention is based on the discovery that genetic and epigenetic parameters and, in particular, the cytosine methylation patterns of said genomic DNAs are particularly suitable for improved treatment and monitoring of breast cell proliferative disorders as well as for the monitoring of a treatment success or treatment failure of said disorders, for example the treatment with tamoxifen. As shown in the appended examples, the present invention is particularly useful in a method for determining the prognosis of a subject with a cell proliferative disorder of the breast tissues and the corresponding selection of a suitable treatment regime.

For example, the monitoring of the methylation status of RASSF1A in a treatment regime with tamoxifen allows for a determination whether said treatment regime is fruitful. As shown in the examples, detection of the RASSF1A-RNA methylation status in, e.g. serum, after a certain period of adjuvant treatment with tamoxifen (or other anti-estrogens) permits the determination/prognosis whether said patient needs further treatment, for example with other therapies, in particular other drugs, medicaments or substances, like aromatase inhibitors. The methods provided herein are also useful in the detection of circulating tamoxifen-resistant cells, for example in blood, serum or NAF.

The nucleic acids according to the present invention can be used for the analysis of genetic and/or epigenetic parameters of genomic DNA.

In another aspect of the present invention, the object of the present invention is achieved using a nucleic acid containing a sequence of at least 18 bases in length of the pretreated genomic DNA according to one of SEQ ID NOs: 6 to 25 and sequences complementary thereto.

The modified nucleic acids could heretofore not be connected with the ascertainment of disease relevant genetic and epigenetic parameters.

The object of the present invention is further achieved by an oligonucleotide or oligomer for the analysis of pretreated DNA, for detecting the genomic cytosine methylation state, said oligonucleotide containing at least one base sequence having a length of at least 10 nucleotides which hybridises to a pretreated genomic DNA according to SEQ ID Nos: 6 to 26. The oligomer probes according to the present invention constitute important and effective tools which, for the first time, make it possible to ascertain specific genetic and epigenetic parameters during the analysis of biological samples for features associated with a patient's response to endocrine treatment. Said oligonucleotides allow the improved treatment and monitoring of breast cell proliferative disorders. The base sequence of the oligomers preferably contains at least one CpG or TpG dinucleotide. The probes may also exist in the form of a PNA (peptide nucleic acid) which has particularly preferred pairing properties. Particularly preferred are oligonucleotides according to the present invention in which the cytosine of the CpG dinucleotide is within the middle third of said oligonucleotide e.g. the 5^{th}-9^{th} nucleotide from the 5′-end of a 13-mer oligonucleotide; or in the case of PNA-oligomers, it is preferred for the cytosine of the CpG dinucleotide to be the 4^{th}-6^{th} nucleotide from the 5′-end of the 9-mer.

The oligomers according to the present invention are normally used in so called “sets” which contain at least two oligomers and up to one oligomer for each of the CpG dinucleotides within SEQ ID NOs: 6 to 26.

In the case of the sets of oligonucleotides according to the present invention, it is preferred that at least one oligonucleotide is bound to a solid phase. It is further preferred that all the oligonucleotides of one set are bound to a solid phase.

The present invention further relates to a set of at least 2 n (oligonucleotides and/or PNA-oligomers) used for detecting the cytosine methylation state of genomic DNA, by analysis of said sequence or treated versions of said sequence (of the genes ESR1, APC, HSD174B4, HIC1 and RASSF1A, as detailed in the sequence listing and Table 1) and sequences complementary thereto). These probes enable improved treatment and monitoring of breast cell proliferative disorders.

The set of oligomers may also be used for detecting single nucleotide polymorphisms (SNPs) by analysis of said sequence or treated versions of said sequence of the genes ESR1, APC, HSD174B4, HIC1 and RASSF1A.

It will be obvious to one skilled in the art that the method according to the invention will be improved and supplemented by the incorporation of markers and clinical indicators known in the state of the art and currently used as diagnostic or prognostic markers. More preferably said markers include node status, age, menopausal status, grade, estrogen and progesterone receptors.

The genes that form the basis of the present invention may be used to form a “gene panel”, i.e. a collection comprising the particular genetic sequences of the present invention and/or their respective informative methylation sites. The formation of gene panels allows for a quick and specific analysis of specific aspects of breast cancer treatment. The gene panel(s) as described and employed in this invention can be used with surprisingly high efficiency for the treatment of breast cell proliferative disorders by prediction of the outcome of treatment with a therapy comprising one or more drugs which target the estrogen receptor pathway or are involved in estrogen metabolism, production, or secretion. The analysis of each gene of the panel contributes to the evaluation of patient responsiveness, however, in a less preferred embodiment the patient evaluation may be achieved by analysis of only a single gene. The analysis of a single member of the ‘gene panel’ would enable a cheap but less accurate means of evaluating patient responsiveness, the analysis of multiple members of the panel would provide a rather more expensive means of carrying out the method, but with a higher accuracy (the technically preferred solution).

According to the present invention, it is preferred that an arrangement of different oligonucleotides and/or PNA-oligomers (a so-called “array”) made available by the present invention is present in a manner that it is likewise bound to a solid phase. This array of different oligonucleotide- and/or PNA-oligomer sequences can be characterised in that it is arranged on the solid phase in the form of a rectangular or hexagonal lattice. The solid phase surface is preferably composed of silicon, glass, polystyrene, aluminium, steel, iron, copper, nickel, silver, or gold. However, nitrocellulose as well as plastics such as nylon which can exist in the form of pellets or also as resin matrices are suitable alternatives.

Therefore, a further subject matter of the present invention is a method for manufacturing an array fixed to a carrier material for the improved treatment and monitoring of breast cell proliferative disorders. In said method at least one oligomer according to the present invention is coupled to a solid phase. Methods for manufacturing such arrays are known, for example, from U.S. Pat. No. 5,744,305 by means of solid-phase chemistry and photolabile protecting groups.

A further subject matter of the present invention relates to a DNA chip for the improved treatment and monitoring of breast cell proliferative disorders. The DNA chip contains at least one nucleic acid according to the present invention. DNA chips are known, for example, in U.S. Pat. No. 5,837,832.

Moreover, a subject matter of the present invention is a kit which may be composed, for example, of a bisulfite-containing reagent, a set of primer oligonucleotides containing at least two oligonucleotides whose sequences in each case correspond to or are complementary to a 18 base long segment of the base sequences specified in SEQ ID NOs: 6 to 26 and/or PNA-oligomers as well as instructions for carrying out and evaluating the described method.

In a further preferred embodiment said kit may further comprise standard reagents for performing a CpG position specific methylation analysis wherein said analysis comprises one or more of the following techniques: MS-SNuPE, MSP, Methyl light, Heavy Methyl, and nucleic acid sequencing. However, a kit along the lines of the present invention can also contain only part of the aforementioned components.

Typical reagents (e.g., as might be found in a typical MethyLight®-based kit) for MethyLight® analysis may include, but are not limited to: PCR primers for specific gene (or methylation-altered DNA sequence or CpG island); TaqMan® probes; optimized PCR buffers and deoxynucleotides; and Taq polymerase.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

The oligomers according to the present invention or arrays thereof as well as a kit according to the present invention are intended to be used for, e.g., the improved treatment monitoring of breast cell proliferative disorders and/or the monitoring of the treatment success of said breast cell proliferative disorders. According to the present invention, the method is preferably used for the analysis of important genetic and/or epigenetic parameters within genomic DNA, in particular for use in improved treatment and monitoring of breast cell proliferative disorders.

The methods according to the present invention are used, for improved detection, treatment and monitoring of breast cell proliferative disorder.

The present invention moreover relates to the diagnosis and/or prognosis of events which are disadvantageous or relevant to patients or individuals in which important genetic and/or epigenetic parameters within genomic DNA, said parameters obtained by means of the present invention may be compared to another set of genetic and/or epigenetic parameters, the differences serving as the basis for the diagnosis and/or prognosis of events which are disadvantageous or relevant to patients or individuals.

In the context of the present invention the term “hybridisation” is to be understood as a bond of an oligonucleotide to a completely complementary sequence along the lines of the Watson-Crick base pairings in the sample DNA, forming a duplex structure.

In the context of the present invention, “genetic parameters” are mutations and polymorphisms of genomic DNA and sequences further required for their regulation. To be designated as mutations are, in particular, insertions, deletions, point mutations, inversions and polymorphisms and, particularly preferred, SNPs (single nucleotide polymorphisms).

In the context of the present invention the term “methylation state” is taken to mean the degree of methylation present in a nucleic acid of interest, this may be expressed in absolute or relative terms i.e. as a percentage or other numerical value or by comparison to another tissue and therein described as hypermethylated, hypomethylated or as having significantly similar or identical methylation status.

In the context of the present invention the term “regulatory region” of a gene is taken to mean nucleotide sequences which affect the expression of a gene. Said regulatory regions may be located within, proximal or distal to said gene. Said regulatory regions include but are not limited to constitutive promoters, tissue-specific promoters, developmental-specific promoters, inducible promoters and the like. Promoter regulatory elements may also include certain enhancer sequence elements that control transcriptional or translational efficiency of the gene.

In the context of the present invention the term “chemotherapy” is taken to mean the use of drugs or chemical substances to treat cancer. This definition includes radiation therapy (treatment with high energy rays or particles), hormone as well as antihormone therapy (treatment with hormones or hormone analogues (synthetic substitutes) and surgical treatment. Accordingly, the invention also provides for a method for the monitoring of a treatment success or a potential treatment success with drugs, radiation or chemical substances to treat cancer. Said treatment protocols and/or regimes comprise, but are not limited to hormonal/antihormonal therapies (e.g. tamoxifen therapies), radiation therapies, antibody therapies (e.g. Herceptin® therapies), chemotherapies (e.g. with cell division/cell cycle inhibitors, like taxol and/or other taxol derivatives) and/or adjuvant therapies (like therapies employing aromatase inhibitors). The treatment protocols and method for monitoring also comprises, in accordance with this invention, the monitoring of chemopreventive strategies (like chemoprevention with, e.g. tamoxifen, aromatase inhibitors or other chemopreventive drugs).

As documented in the appended examples, in particular the measurement/detection of the methylation status of RASSF1A is particular useful in the determination of a treatment prognosis and/or a treatment success with hormonal/antihormonal therapies, in particular in a tamoxifen therapy. As known in the art, tamoxifen is a selective estsrogen receptor modulator with anti-estrogenic activity in the breast and estrogenic-like activity in the endometrium, bone and lipid metabolism; see, e.g. Baselga (2002), Cancer Cell 1, 319-322.

In the context of the present invention, “epigenetic parameters” are, in particular, cytosine methylations and further modifications of DNA bases of genomic DNA and sequences further required for their regulation. Further epigenetic parameters include, for example, the acetylation of histones which, cannot be directly analysed using the described method but which, in turn, correlates with the DNA methylation.

In the following, the present invention will be explained in greater detail on the basis of the sequences, figures and examples without being limited thereto.

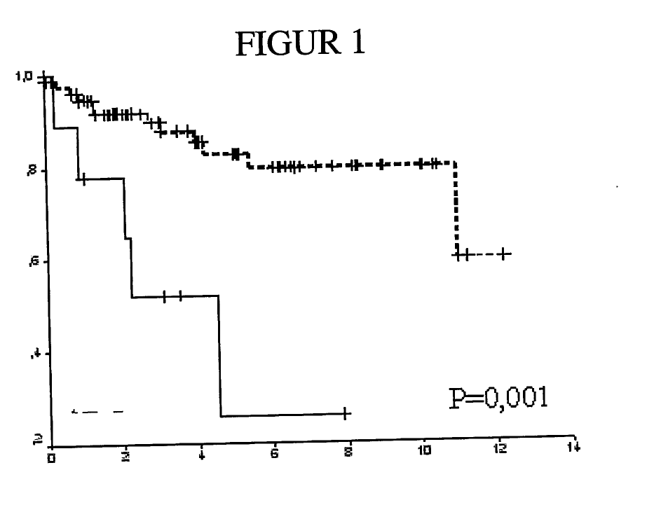

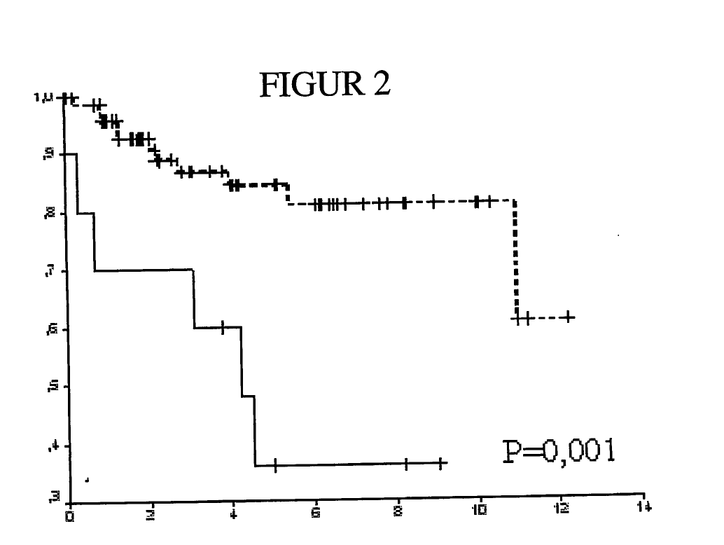

FIG. 1 shows the Kaplan-Meier estimated overall survival curves for the gene APC, for a set of 86 breast cancer patients. The dotted line (upper curve) shows unmethylated samples whereas the unbroken line (lower curve) shows methylated samples. The x-axis shows the number of years, and the Y-axis shows the proportion of the group.

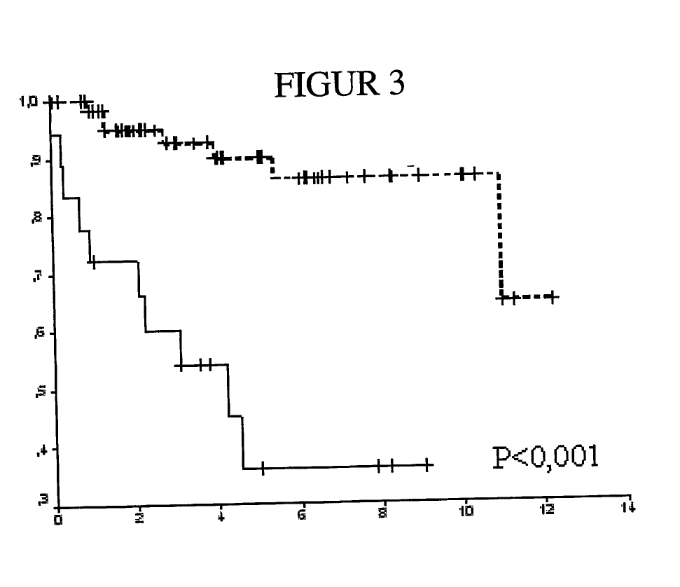

FIG. 2 shows the Kaplan-Meier estimated overall survival curves for the gene RASSF1A, for a set of 86 breast cancer patients. The dotted line (upper curve) shows unmethylated samples whereas the unbroken line (lower curve) shows methylated samples. The x-axis shows the number of years, and the Y-axis shows the proportion of the group.

FIG. 3 shows the combined Kaplan-Meier estimated overall survival curves for the genes APC and/or RASSF1A, for a set of 86 breast cancer patients. The dotted line (upper curve) shows unmethylated samples whereas the unbroken line (lower curve) shows methylated samples. The x-axis shows the number of years, and the Y-axis shows the proportion of the group.

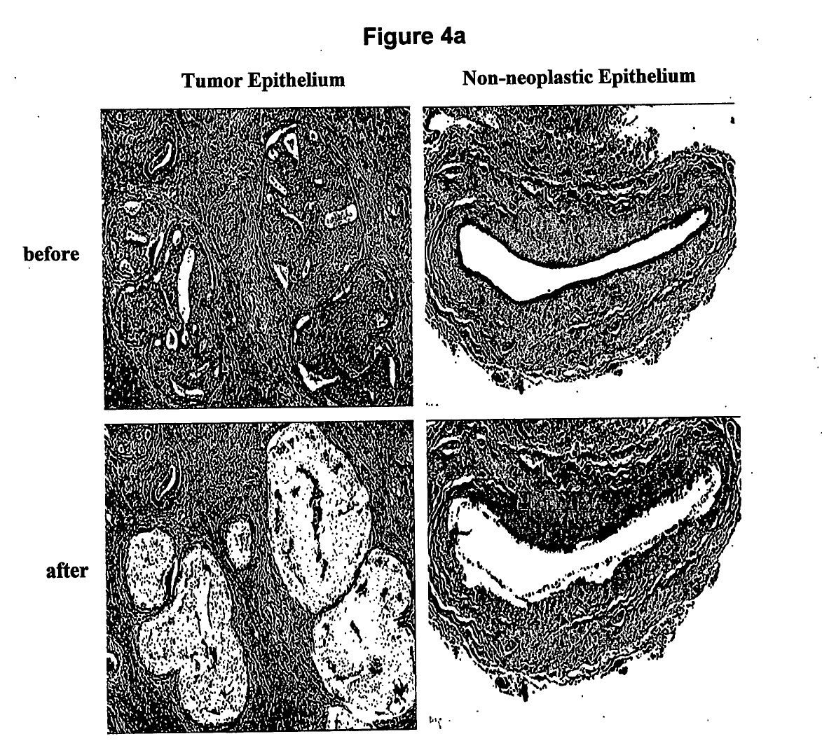

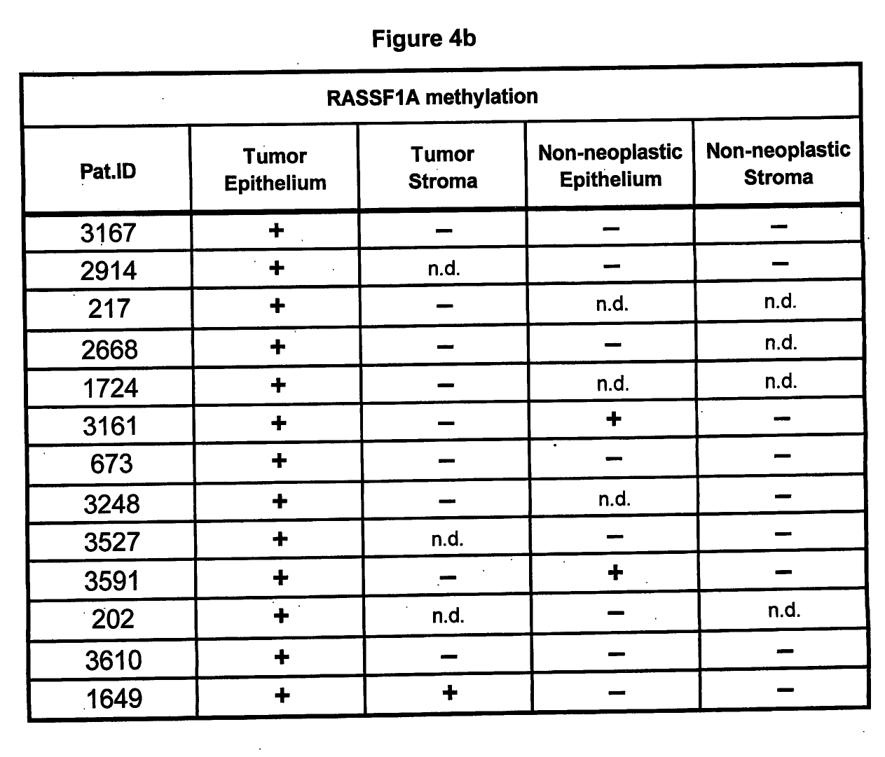

FIG. 4. RASSF1A methylation in microdissected cells.

(a) Tumor and non-neoplastic epithelial cells before and after microdissection. Original magnification, ×40. (b) Overview of RASSF1A methylation status in tumor and non-neoplastic tissue. +, PMR value >0; −, PMR value =0; n.d. not determined, because no DNA could be extracted.

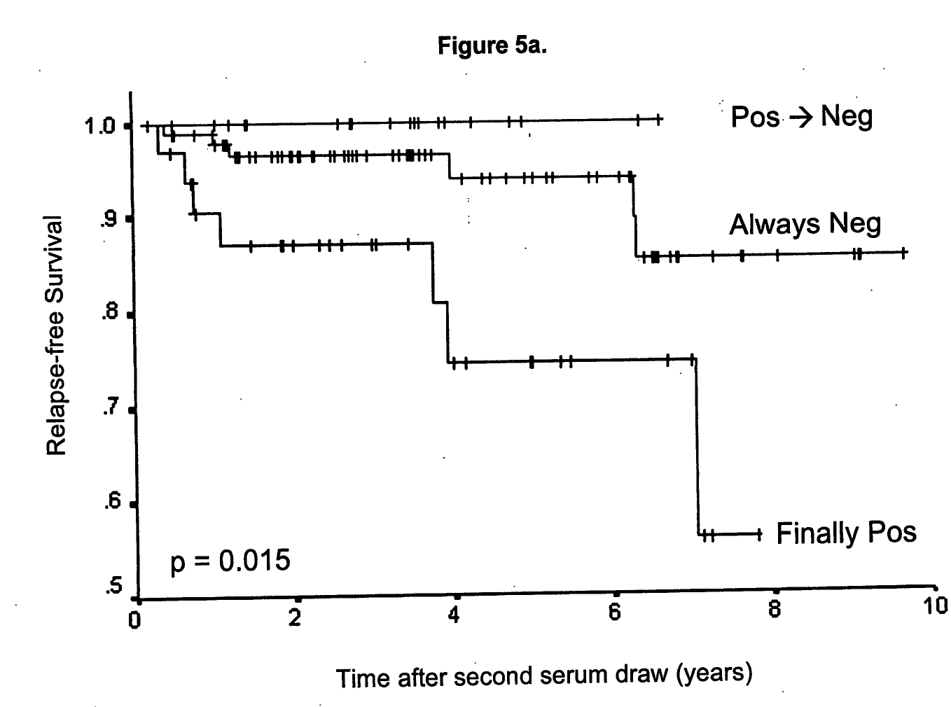

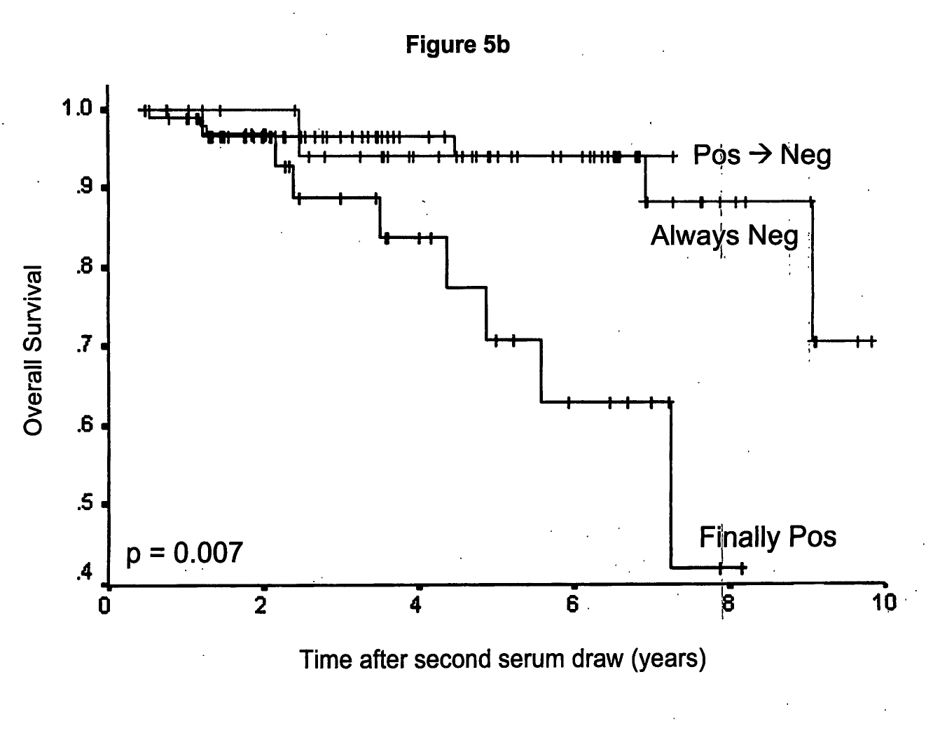

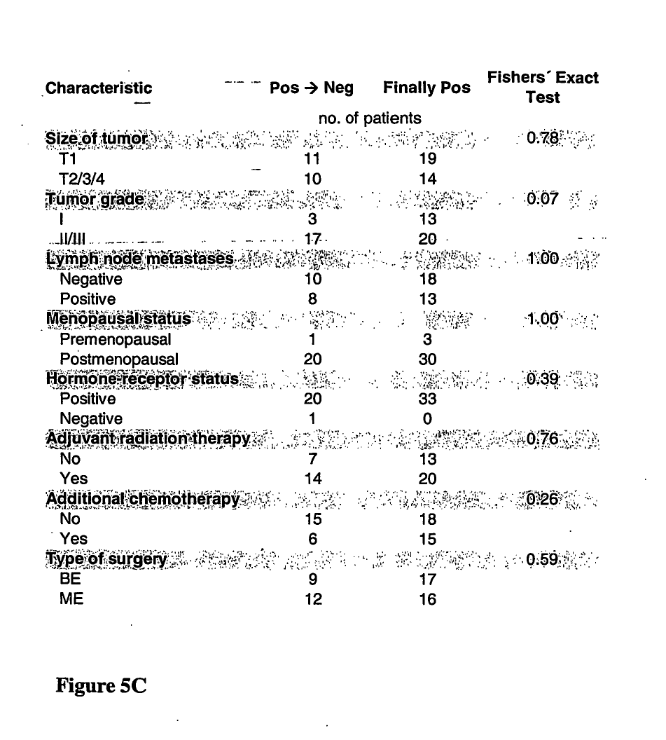

FIG. 5. Survival and changes in RASSF1A DNA methylation status. (a) Relapse-free and (b) overall survival according to RASSF1A methylation status in serum that switched from positive to negative, stayed always negative or was finally positive after one year of tamoxifen treatment. (c) Characteristics of those patients according to the RASSF1A methylation status.

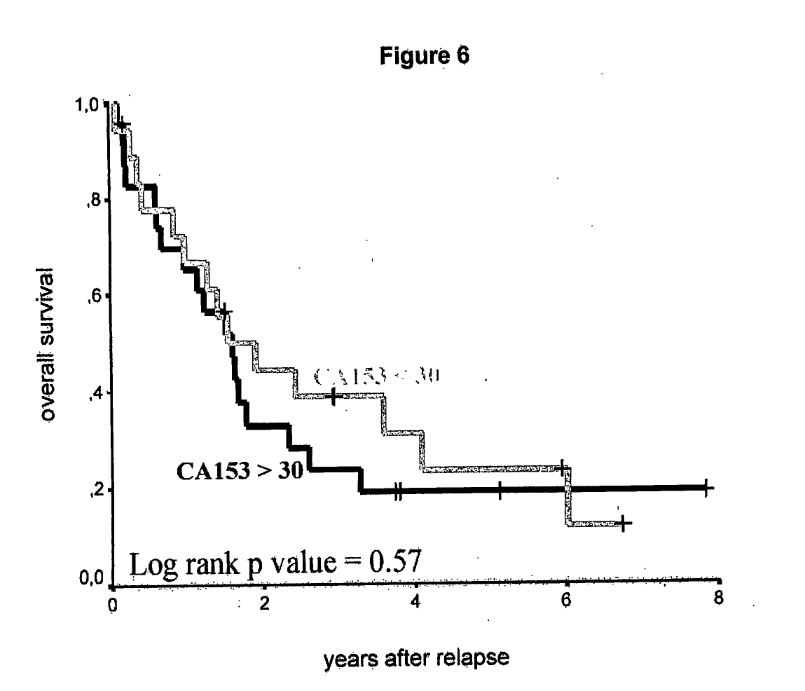

FIG. 6 Overall survival depending on CA153 level in sera collected immediately before diagnosis of relapse

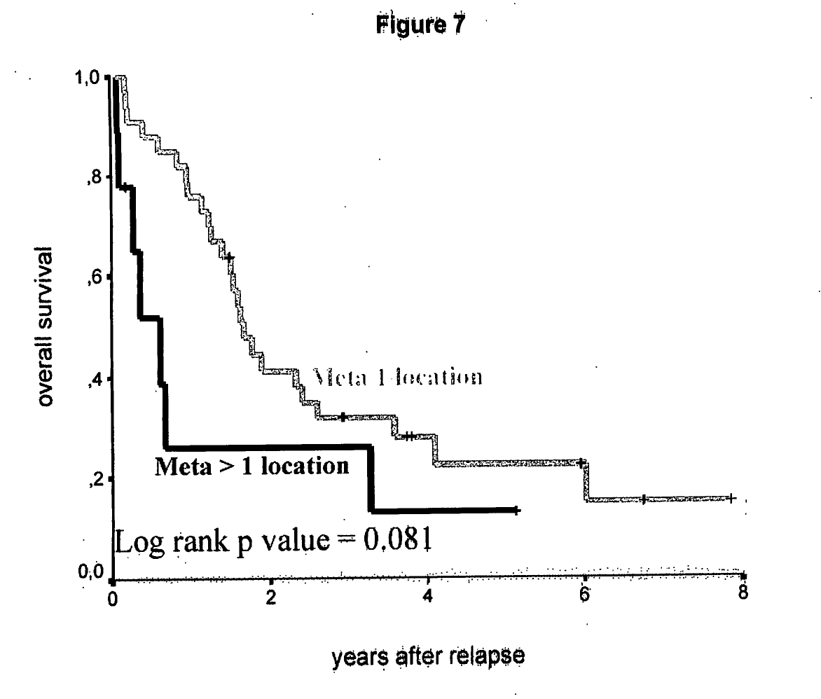

FIG. 7 Overall survival depending on the number of locations of metastasis

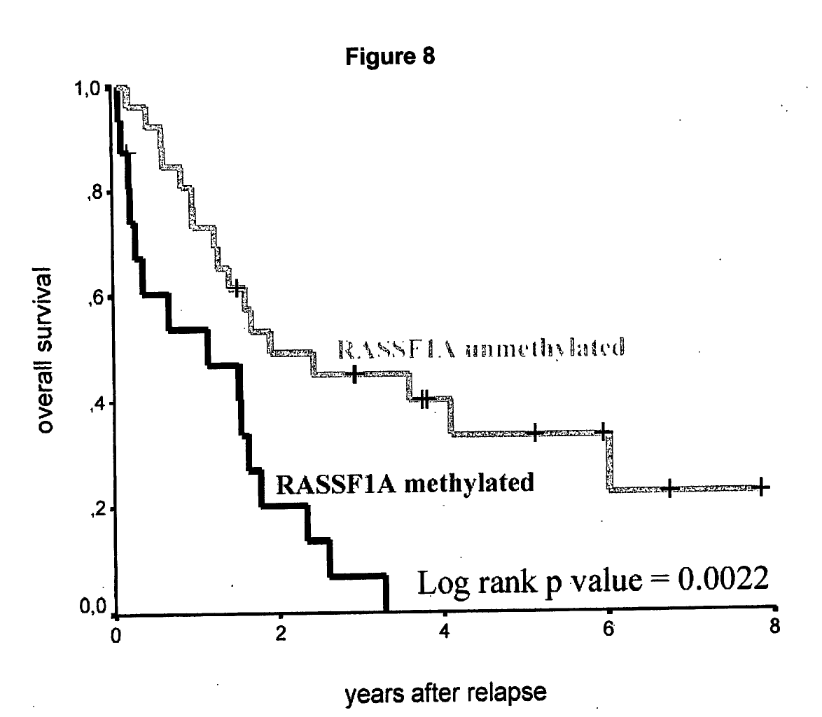

FIG. 8 Overall survival depending on RASSF1A DNA methylation status in sera collected immediately before diagnosis of relapse.

SEQ ID NOs: 1 to 5 represent 5′ and/or regulatory regions and/or CpG rich regions of the genes according to Table 1. These sequences are derived from Genbank and will be taken to include all minor variations of the sequence material which are currently unforeseen, for example, but not limited to, minor deletions and SNPs.

SEQ ID NOs: 6 to 26 exhibit the pretreated sequence of DNA derived from the genomic sequence according to Table 1. These sequences will be taken to include all minor variations of the sequence material which are currently unforeseen, for example, but not limited to, minor deletions and SNPs.

SEQ ID NOs. 27 to 31: Primer and probe sequences for ACTB were 5′-TGGTGATGGAGGAGGTTTAGTAAGT-3′ (forward primer; SEQ ID NO: 26), 5′-AACCAATAAAACCTACTCCTCCCTTAA-3′ (reverse primer; SEQ ID NO: 27) and 5′-FAM-ACCACCACCCAACACACAATAACAAACACA-BHQ1-3′ (probe; SEQ ID NO: 28), for methylated RASSF1A 5′-ATTGAGTTGCGGGAGTTGGT-3′ (forward primer; SEQ ID NO: 29), 5′-ACACGCTCCAACCGAATACG-3′ (reverse primer; SEQ ID NO: 30) and 5′-FAM-CCCTTCCCAACGCGCCCA-BHQ1-3′ (probe; SEQ ID NO: 31).

### EXAMPLES

### Example 1

### Gene Identification and Assessment

Using MethyLight, a high-throughput DNA methylation assay, the inventors analysed 39 genes in a gene evaluation set, consisting of ten sera from metastasised patients, 26 patients with primary breast cancer and ten control patients. In order to determine the prognostic value of genes identified within the gene evaluation set, the inventors finally analysed pretreatment sera of 24 patients having had no adjuvant treatment (training set) to determine their prognostic value. An independent test set consisting of 62 patients was then used to test the validity of genes and combinations of genes, which in the training set were found to be good prognostic markers.

In the gene evaluation set the inventors identified five genes (ESR1, APC, HSD17B4, HIC1 and RASSF1A). In the training set, patients with methylated serum DNA for RASSF1A and/or APC had the worst prognosis (p<0.001). This finding was confirmed by analysing serum samples from the independent test set (p=0.007). When analysing all 86 investigated patients, multivariate analysis showed methylated RASSF1A and/or APC serum DNA to be independently associated with poor outcome, with a relative risk for death of 5.7. DNA methylation of particular genes in pretherapeutic sera of breast cancer patients, especially of RASSF1A/APC, is more powerful than standard prognostic parameters.

The gene evaluation set consisted of patients with recurrent disease (n=10; sera obtained at diagnosis of metastasis in the bone, lung, brain or liver) and pretherapeutic sera of recently diagnosed primary breast cancer patients (n=26; age range: 36.1 yrs to 83.9 yrs. (mean: 59.3 yrs.); two, 18 and six patients had pT1, pT2 and pT3 cancers, respectively; 15, ten and one patients had lymph node-negative, -positive and unknown disease, respectively) and normal controls (n=10; age range: 20.5 to 71.5 yrs. (mean: 44.6 yrs.); all underwent a core biopsy and were confirmed to have benign disease of the breast).

To assess prognostic significance the inventors used pretherapeutic sera in independent training (n=24) and test (n=62) sets consisting of patients who did not receive any adjuvant treatment after surgery.

Systemic adjuvant therapy was either not necessary or the patients were not eligible or refused any further treatment. The primary surgical procedure included breast-conserving lumpectomy or modified radical mastectomy and axillary lymph node dissection. Median age of the study population was 60 years (range, 28 to 86 yrs.). After a median follow-up of 3.7 yrs. (range: one month to 12.2 yrs.) 17 of the 86 patients (20%) had died. Distribution of aberrant serum DNA methylation of the 86 patients and association with clinical and histopathological characteristics are shown in Table 2.

Patients' blood samples were drawn prior to therapeutic intervention. The blood was centrifuged at 2000 g for 10 min at room temperature, and 1 mL aliquots of serum samples were stored at −30° C. Genomic DNA from serum samples was isolated using the High Pure Viral Nucleic Acid Kit (Roche Diagnostics, Mannheim, Germany) according to the manufacturer's protocol with some modifications for multiple loading of the DNA extraction columns to gain a sufficient amount of DNA. Thus, 4×200 μl of a serum sample were each mixed with 200 μl of working solution (binding buffer supplemented with polyA carrier RNA) and 50 μl proteinase K [18 mg/ml] and incubated for 10 minutes at 72° C. After adding 100 μl isopropanol the solution was mixed, loaded onto the extraction column and centrifuged for 1 minute at 8000 g. The flow-through was pipetted back into the same column reservoir and centrifuged a second time. This procedure was repeated four times for each serum sample. After these “pooling steps” the DNA isolation was processed as described in the manufacturer's protocol. For DNA elution 55 μl of AE-buffer (Quiagen, Calif., USA) were added, incubated for 20 min at 45° C. and centrifuged for three minutes at 12.000 g. For both, normal sera and cancer sera analysis the same amount of serum for DNA extraction was used.

Sodium bisulfite conversion of genomic DNA was performed as described previously (Eads et al.: MethyLight: a high-throughput assay to measure DNA methylation. Nucleic Acids Res., 28: E32, 2000).

Sodium bisulfite-treated genomic DNA was analysed by means of the MethyLight, a fluorescence-based, real-time PCR assay, as described previously (Eads et al 2000, see above, Eads et al.: Epigenetic patterns in the progression of esophageal adenocarcinoma. Cancer Res., 61: 3410-3418, 2001). Briefly, two sets of primers and probes, designed specifically for bisulfite-converted DNA, were used: a methylated set for the gene of interest and a reference set, β-actin (ACTB), to normalise for input DNA. Serum samples of patients with recurrent disease revealed the highest amount of β-actin, whereas no difference between β-actin values from serum samples of patients with primary breast cancer and sera of normal controls was observed. Specificity of the reactions for methylated DNA was confirmed separately using SssI (New England Biolabs)-treated human white blood cell DNA (heavily methylated). The percentage of fully methylated molecules at a specific locus was calculated by dividing the GENE:ACTB ratio of a sample by the GENE:ACTB ratio of SssI-treated white blood cell DNA and multiplying by 100. The abbreviation PMR (percentage of fully methylated reference) indicates this measurement. For each MethyLight reaction 10 μl of bisulfite-treated genomic DNA was used.

A gene was deemed methylated if the PMR value was >0. Primer and probes specific for methylated DNA and used for MethyLight reactions are listed in Supplemental Data.

The inventors used Pearsons Chi² or—in the case of low frequencies per cell—Fisher's exact method to test associations between categorically clinicopathological features. The Mann-Whitney-U-Test was used to assess differences between non-parametric distributed variables. Overall survival was calculated from the date of diagnosis of the primary tumour to the date of death or last follow-up. Overall survival curves were calculated with the Kaplan-Meier method. Univariate analysis of overall survival according to clinicopathological factors (histological type, tumour stage, nodal status, grading, menopausal status, hormone receptor status (estrogen and/or progesterone receptor positively), estrogen and progesterone receptor status) and gene methylation were performed using a two-sided log-rank test.

Multivariate Cox proportional hazards analysis was used to estimate the prognostic effect of methylated genes.

A p value <0.05 was considered a statistically significant difference. All statistical analyses were performed using SPSS Software 10.0.

The inventors initially investigated 39 genes in the sera of ten patients with metastasised breast cancer for the presence of aberrant methylation. The 33 genes positive in the sera of the metastasised patients were further evaluated in an independent sample set of pretherapeutic sera of 26 patients with primary breast cancer and ten healthy controls. An overview of the frequency of methylation in the investigated serum samples is given in Table 3. The most appropriate genes for our further analyses were determined to be those that met one of the following criteria: (i) unmethylated in serum samples from healthy controls and >10% methylated in serum samples from primary breast cancer patients, or (ii)≦10% methylated in serum samples from healthy controls and >20% methylated in serum samples from primary breast cancer patients. A total of five genes, namely ESR1, APC, HSD17B4, HIC1 and RASSF1A, met at least one of these criteria (Table 3).

Pre-treatment serum samples from patients included in the training set were used to evaluate the prognostic value of the methylation status of these five genes. In this training set the inventors identified ESR1, APC or RASSF1A methylation in primary breast cancer patients' sera to be markers of poor prognosis, whereas HSD17B4 reached only borderline significance and aberrant methylation of HIC1 showed no significant results (Table 4). Furthermore, various combinations of the investigated genes were analyzed. Patients were classified as methylation-positive if at least one of the genes included in the combination showed aberrant methylation. Patients with methylated serum DNA for RASSF1A and/or APC had the worst prognosis (P<0.001), even worse than when each gene was analysed individually (Table 4).

The highly significant prognostic value for APC and/or RASSF1A methylation in serum samples from breast cancer patients was confirmed by analysing the test set (P=0.007, log rank test). ESR1 and APC methylation as single genes or the combinations ESR1/RASSF1A and ESR1/APC no longer had prognostic significance (Table 4).

Combined analysis of the training and test sets (n=86) showed correlation between ESR1 and RASSF1A (P=0.005) and between ESR1 and APC (P=0.031), whereas no correlation was observed between RASSF1A and APC. In patients with advanced tumours RASSF1A and ESR1 methylation and in patients with progesterone receptor-negative tumours APC methylation was more prevalent in pretherapeutic sera, while no further associations were seen between clinicopathological features and DNA methylation of APC, ESR1 or RASSF1A (Table 5). RASSF1A methylation in pretherapeutic sera was more prevalent in older than in younger patients, whereas age had no effect on DNA methylation of ESR1 or APC.

Univariate analysis of all 86 investigated patients (training set plus test set) revealed prognostic significance for tumour size, lymph node metastases and methylation status of APC, RASSF1A and the combination of RASSF1A/APC (Table 6; FIG. 1). Due to the fact that ESR1 methylation correlates with APC as well as with RASSF1A methylation, the inventors did not test the triple combination in the univariate or the multivariate analyses of all 86 patients.

The Cox multiple-regression analysis included tumour size, lymph node metastases, age and methylation status of the investigated genes. Beside lymph node status, methylated RASSF1A and/or APC serum DNA was strongly associated with poor outcome, with a relative risk for death of 5.7 (Table 7).

Prognosis in patients with newly diagnosed breast cancer is determined primarily by the presence or absence of metastases in draining axillary lymph nodes. Nevertheless, the life-threatening event in breast cancer is not lymph node metastasis per se, but haematogenous metastases which mainly affect bone, liver, lung and brain. The inventors therefore aimed to develop a prognostic test that is sensitive for haematogenous metastases and could be performed in patients' pretherapeutic serum.

In recent years several studies have reported cell-free tumour-specific DNA in serum/plasma of breast cancer patients at diagnosis. Aberrant methylation of serum/plasma DNA of patients with various types of malignancies, including breast cancer, has been described (see above).

In light of these observations, the inventors examined the methylation status of 39 genes, which, on the one hand, are known to be frequently methylated in breast cancer and other malignancies (Jones and Baylin: The fundamental role of epigenetic events in cancer. Nat. Rev. Genet., 3: 415-428, 2002; Widschwendter and Jones: DNA methylation and breast carcinogenesis. Oncogene, 21: 5462-5482, 2002) and, on the other hand, were reported to be abnormally regulated in tumours of patients with poor prognostic breast cancer (van't Veer et al.: Gene expression profiling predicts clinical outcome of breast cancer. Nature, 415: 530-536, 2002; van de Vijver et al.: A gene-expression signature as a predictor of survival in breast cancer. N. Engl. J. Med., 347: 1999-2009, 2002) Because levels of circulating DNA in metastasised patients are known to be higher (Leon et al.: Free DNA in the serum of cancer patients and the effect of therapy Cancer Res., 37: 646-650, 1977) and because the loss of genetic heterogeneity of disseminated tumour cells with the emergence of clinically evident metastasis was recently reported (Klein et al.: Genetic heterogeneity of single disseminated tumour cells in minimal residual cancer. Lancet, 360: 683-689, 2002), the inventors firstly investigated these 39 genes in ten sera of metastasised patients to determine the overall prevalence of methylation changes in breast cancer. As a next step the inventors analysed the 33 genes that were positive in the metastasised patients, in the pre-treatment sera of 26 patients with primary breast cancer and in ten benign controls in order to identify the most important genes for further analysis. Eventually the inventors came up with five genes (ESR1, APC, HSD17B4, HIC1 and RASSF1A), which were primarily analysed in a group of 24 patients (training set). To confirm the significance of this result the inventors tested these genes in an independent set of 62 patients (test set). In order to apply the strictest criteria for testing the potential of a prognostic factor, the inventors investigated these markers in women, who had not undergone adjuvant systemic treatment. DNA methylation of APC and RASSF1A in pre-therapeutic sera, both frequently methylated and abnormally regulated in human primary breast cancers (Dammann et al.: Hypermethylation of the cpG island of Ras association domain family 1A (RASSF1A), a putative tumor suppressor gene from the 3p21.3 locus, occurs in a large percentage of human breast cancers. Cancer Res., 61: 3105-3109, 2001; Virmani et al.: Aberrant methylation of the adenomatous polyposis coli (APC) gene promoter 1A in breast and lung carcinomas. Clin. Cancer Res., 7: 1998-2004, 2001), turned out to be a strong independent prognostic parameter. These genes are involved in pathways counteracting metastasis: mediation of intercellular adhesion, stabilisation of the cytoskeleton, regulation of the cell cycle and apoptosis (Fearnhead et al.: The ABC of APC. Hum. Mol. Genet., 10: 721-733, 2001; Dammann et al.: Epigenetic inactivation of the Ras-association domain family 1 (RASSF1A) gene and its function in human carcinogenesis. Histol. Histopathol., 18: 665-677, 2003). Methylated DNA in patients' pretherapeutic serum coding for these two genes reflects poor prognosis. The source of the tumour-specific DNA and its definite role in metastasis remains elusive. Circulating tumour-specific altered genetic information may serve as a surrogate marker for circulating tumour cells that ultimately cause distant metastases. An alternative, but equally attractive, hypothesis is that circulating altered DNA per se may cause de novo development of tumour cells in organs known to harbour breast cancer metastases. This so-called “Hypothesis of Genometastasis” suggests that malignant transformation might develop as a result of transfection of susceptible cells in distant target organs with dominant oncogenes that circulate in the plasma and are derived from the primary tumour. Interestingly, irrespective of the source of DNA in the serum, it is noteworthy that some genes provide prognostic information when methylated in patients' sera, whereas genes like HIC1, which is methylated in about 40% and 90% of primary and metastasised breast cancer patients, respectively, but in only 10% of healthy individuals, are not at all a prognostic parameter.

Irrespective of the mechanistic role of methylated DNA with regards to metastasis in breast cancer patients, these epigenetic changes in serum have several advantages as indicators of poor prognosis as compared to currently used or studied prognostic parameters: DNA in serum is stable and can be analysed by a high-throughput method like MethyLight Compared to bone marrow aspiration, a simple blood draw (which can be repeated any time throughout the follow-up period) is sufficient. The more screening mammographies are performed, the more small cancers are treated and after histopathological examination no tumour material will remain to perform RNA- and/or protein-based assays for risk evaluation. This application therefore demonstrates a useful and easy approach for risk assessment of breast cancer patients.

### Example 2

### Circulating Tumor-Specific DNA

### A Marker for Monitoring Efficacy of Adjuvant Therapy in Cancer Patients

Adjuvant systemic therapy (a strategy that targets potential disseminated tumor cells after complete removal of the tumor) has clearly improved survival of cancer patients. Up to date no tool is available to monitor efficacy of these therapies, unless distant metastases arise, a situation that leads unavoidably to death.

RASSF1A methylation is shown herein as a DNA-based marker for circulating breast cancer cells, in particular said presence of RASSF1A methylation in the great majority of invasive breast cancer specimens, that are mainly observed in breast cancer cells but rarely in other compartments of the tumor or the remaining breast and since a low frequency of RASSF1A DNA methylation in pretherapeutic serum samples from non-breast cancer individuals is observed ( 11/154, 5/93 and 3/78 patients with benign conditions of the breast, primary cervical cancer or prostate cancer, respectively, had RASSF1A methylated).

To assess the capability whether this breast cancer-specific markers is able to monitor adjuvant treatment, we analyzed RASSF1A DNA methylation in pretherapeutic sera and serum samples collected one year after surgery from 148 breast cancer patients who were receiving adjuvant tamoxifen. 19.6% and 22.3% of breast cancer patients showed RASSF1A DNA methylation in their pretherapeutic and one-year after serum samples, respectively. As documented herein below RASSF1A methylation one year after primary surgery (and during adjuvant tamoxifen therapy) was an independent predictor of poor outcome, with a relative risk for relapse of 5.1 (1.3-19.8) and for death of 6.9 (1.9-25.9).

Surprisingly, measurement of serum DNA methylation permits adjuvant systemic treatment to be monitored for efficacy: Disappearance of RASSF1A DNA methylation in serum throughout treatment with tamoxifen indicates a response, while persistence or new appearance means resistance to adjuvant tamoxifen treatment.

Breast cancer is the most frequent malignancy among women in the industrialized world. Although the presence or absence of metastatic involvement in the axillary lymph nodes is the most powerful prognostic factor available for patients with primary breast cancer (Goldhirsch, (2001) J. Clin. Oncol. 19, 3817-3827), it is only an indirect measure reflecting the tumor's tendency to spread. About 75% of breast cancers are hormone-dependent, and the postoperative administration of tamoxifen reduces the risk of recurrence by 47 percent and reduces the risk of death by 26 percent Early Breast Cancer Trialists' Collaborative Group, (1998) Lancet 351, 1451-1467). Tamoxifen, which is both an antagonist and a partial agonist of the estrogen receptor (Riggs, (2003), N. Engl. J. Med. 348, 618-629), is usually administered for five years to women with hormone-receptor-positive breast cancers to target disseminated tumor cells. Recent evidence from large trials demonstrates significant improvement of disease-free survival by administering letrozole or examestane, both aromatase inhibitors, after completing five or two to three years of standard tamoxifen treatment, respectively (Coombes, (2004) N. Engl. J. Med. 350, 1081-1092; Goss, (2003) N. Engl. J. Med. 349, 1793-1802). However, the absolute benefits are limited: One event per year per 100 women treated can be reduced by letrozole. Not only did a large majority of these patients not profit from this secondary adjuvant treatment but they also experienced considerably high costs as well as toxic effects like hot flashes, arthritis, arthralgia, and myalgia. Induction of osteoporosis by long-term administration of aromatase inhibitors is an additional risk. For future secondary adjuvant treatment studies, a highly sensitive marker for tamoxifen-resistant circulating cells is urgently needed. Such a marker should preferably fulfill certain requirements: (i) absence in non-breast cancer patients, (ii) easy availability and measurability in patients throughout follow-up period without discomfort or harm, (iii) poor prognostic parameter in non-systemically treated patients, (iv) identification of patients during adjuvant treatment who are non-responsive to endocrine therapy used.

As documented herein above APC and RASSF1A methylation in pre-therapeutic sera of breast cancer patients has high prognostic value. In particular, RASSF1A DNA methylation has herein above been shown to be a prognostic marker in patients who did not receive adjuvant therapy.

The following experiments document/demonstrate that methylated RASSF1A DNA in serum is a surrogate marker for circulating breast cancer cells and that this cancer-specific DNA alteration allows monitoring of adjuvant therapy in cancer patients: Disappearance of RASSF1A DNA methylation in serum throughout treatment with tamoxifen indicates a response, while persistence or new appearance means resistance to adjuvant tamoxifen treatment.

### Material and Methods

### Patients

Pre- and posttherapeutic serum samples of 148 breast cancer patients were studied. Serum samples from our serum bank were recruited from all patients diagnosed with breast cancer between September 1992 and February 2002, who met all the following criteria: primary breast cancer without metastasis at diagnosis, tamoxifen treatment for a total of five years or upon relapse, availability of serum samples before treatment and one year after treatment (a time when the patient has received at least six monthly adjuvant treatments with tamoxifen 20 mg per day) and no relapse after one year. Patient characteristics are shown in Table 9. Patients were 37 to 88 years old (median age at diagnosis, 62 years). After a median follow-up (after the second serum draw) of 3.6 yrs. (range: 0.2 to 9.7 yrs.) and 4.0 yrs. (range: 0.5 to 9.8 yrs.) Seven (4.7%) and eight (5.4%) patients had relapsed or died, respectively. Throughout the entire observation period, 13 (8.8%) and 15 (10.1%) patients relapsed or died, respectively. Hormone receptor status was determined by either radioligand binding assay or immunohistochemistry.

In addition, serum samples from 154 patients with benign condition of the breast, from 93 patients with cervical cancer and 78 patients with prostate cancer have been analyzed.

### Serum Samples and DNA Isolation

Patients' blood samples were drawn prior to or one year after therapeutic intervention, respectively. Blood was centrifuged at 2000 g for 10 min at room temperature, and 1 ml aliquots of serum samples were stored at −30° C.

Genomic DNA from serum was isolated using a QIAmp tissue kit (Qiagen, Hilden, Germany) and the High Pure Viral Nucleic Acid Kit (Roche Diagnostics, Mannheim, Germany), respectively, according to the manufacturers' protocol and some modifications described above.

### Laser-Capture Microdissection.

The PixCell II LCM System (Arcturus Engineering, Mountain View, Calif.) was used for LCM of paraffin-embedded tissues. 10-μm-thick sections of 13 breast cancer patients with a ductal carcinoma in situ (DCIS) were used. For each analyzed fraction 1000 cells were “laser captured”. DNA extraction was carried out using the Arcturus Pico Pure DNA extraction Kit according to the manufacturers' instructions.

### Analysis of DNA Methylation

Sodium bisulfite conversion of genomic DNA was performed as described previously. Sodium bisulfite-treated genomic DNA was analyzed by means of MethyLight, a fluorescence-based, real-time PCR assay, as described previously (17, 18). Briefly, two sets of primers and probes designed specifically for bisulfite-converted DNA were used: a methylated set for the gene of interest and a reference set, β-actin (ACTB), to normalize for input DNA. Specificity of the reactions for methylated DNA was conformed separately using SssI (New England Biolabs)-treated human genomic DNA (heavily methylated). Dividing the GENE:ACTB ratio of a sample by the GENE:ACTB ratio of SssI-treated genomic DNA and multiplying by 100 calculated the percentage of fully methylated molecules at a specific locus. The abbreviation PMR (percentage of fully methylated reference) indicates this measurement. For each MethyLight reaction 10 μl of bisulfite-treated genomic DNA was used.

A gene analyzed in serum DNA was deemed methylated if the PMR value was >0. Primer and probe sequences for ACTB were 5′-TGGTGATGGAGGAGGTTTAGTAAGT-3′ (forward primer; SEQ ID NO: 26), 5′-AACCAATAAAACCTACTCCTCCCTTAA-3′ (reverse primer; SEQ ID NO: 27) and 5′-FAM-ACCACCACCCAACACACA ATAACAAACACA-BHQ1-3′ (probe; SEQ ID NO: 28), for methylated RASSF1A 5′-ATTGAGTTGCGGGAGTTGGT-3′ (forward primer; SEQ ID NO: 29), 5′-ACACGCTCCAACCGAATACG-3′ (reverse primer; SEQ ID NO: 30) and 5′-FAM-CCCTTCCCAACGCGCCCA-BHQ1-3′(probe; SEQ ID NO: 31).

### Statistics

Pearson's Chi² or, in the case of low frequencies per cell, Fisher's exact method to test associations between categorically clinicopathological features and methylation measures were used. The Mann-Whitney U Test was used to assess differences between non-parametric distributed variables. Relapse-free and overall survival were calculated from the date of second serum draw (one year after diagnosis) to the date of relapse or death or last follow-up. Relapse-free and overall survival curves were calculated with the Kaplan-Meier method. Univariate analysis of overall survival according to clinicopathological factors (tumor stage, grading, nodal status, menopausal status, hormone receptor status (estrogen and/or progesterone receptor positivity)) and pretherapeutic and one-year-after serum RASSF1A DNA methylation was performed using a two-sided log-rank test.

Multivariate Cox proportional hazards analysis was used to estimate the predictive effect of methylated serum RASSF1A DNA.

A p value <0.05 was considered a statistically significant difference. All statistical analyses were performed using SPSS Software 10.0.

### Results

### RASSF1A DNA Methylation in Laser-Capture Microdissected Breast Cancer Cells

The rationale for supposing RASSF1A methylation as a DNA-based marker for breast cancer cells was based on our previous finding that 98.6% of 148 analyzed breast cancer specimens showed positive PMR values for RASSF1A DNA methylation, as documented above and that RASSF1A methylation in pretherapeutic serum samples of breast cancer patients who did not receive any systemic adjuvant therapy was an independent poor prognostic marker.

In order to fully document that RASSF1A DNA methylation acts as a DNA-based marker solely for breast cancer cells but not for other breast- and/or tumor-associated cells, we performed laser-assisted microdissection of 13 paraffin-embedded specimens that had been removed due to hormone receptor positive carcinoma in situ. RASSF1A methylation was detected in all cancer cell fractions, whereas the large majority of the underlying stroma or the non-neoplastic breast epithelium or the adjacent stroma were negative for RASSF1A methylation (FIG. 4).

### RASSF1A DNA Methylation in Serum of Non-Breast Cancer Patients

To assess whether RASSF1A DNA methylation in serum is a breast cancer-specific marker, we analyzed pretherapeutic sera from non-breast cancer: RASSF1A DNA methylation (PMR values >0) was detectable in pretherapeutic serum samples from only 11/154 (7.1%), 5/93 (5.4%) and 3/78 (3.8%) patients with benign conditions of the breast, primary cervical cancer and prostate cancer, respectively. These findings substantiate the conjecture that RASSF1A methylation in serum is a specific marker for circulating breast cancer cells.

RASSF1A DNA Methylation in Serum of Adjuvantly Tamoxifen-Treated Patients with Primary Breast Cancer

In this retrospective approach we used prospectively collected serum samples from patients who received tamoxifen for adjuvant treatment due to primary non-metastatic breast cancer, who had pretherapeutic as well as serum samples drawn one year after diagnosis (i.e. >six months after start of tamoxifen therapy) and who showed no relapse within the first year after diagnosis or at second serum draw. A total of 19.6% and 22.3% of patients showed RASSF1A DNA methylation in their pretherapeutic and one-year-after serum samples, respectively. Pretherapeutic RASSF1A methylation showed nearly the same associations with clinicopathological parameters as described earlier for a different set of patients (17) and was correlated with tumor size, menopausal status (Table 10) and age (median age: RASSF1A unmethylated (59.7 yrs; 36.9-88.4); RASSF1A methylated (67.6 yrs; 45.8-85.3; P=006)). RASSF1A DNA methylation at second serum draw after one year (Table 10) was associated only with age (median age: RASSF1A unmethylated (61.3 yrs; 37.8-86.1); RASSF1A methylated (67.4 yrs; 45.2-89.6; P=0.047)).

### Prognostic Significance of Clinicopathological Features and Pretherapeutic RASSF1A DNA Methylation in Serum

Tumor size as well as lymph node metastasis were poor prognostic parameters for relapse-free as well as for overall survival, whereas tumor grade had a statistically significant impact on relapse-free survival (Tables 11A and 11B). Neither menopausal status, HR status nor pretherapeutic RASSF1A DNA methylation in serum had an impact on prognosis (Tables 11A and 11B).

### Early Identification of Patients Who are Non-Responsive to Adjuvant Tamoxifen

About one year (1.04+/−0.11 yr.) after primary diagnosis of breast cancer (after patients were on tamoxifen 20 mg daily for at least six months), a second serum draw was done. Serum RASSF1A DNA methylation at that time indicated poor relapse-free as well as overall survival (Tables 11A and 11B). To test whether serum RASSF1A DNA methylation is an independent predictor of non-responsiveness to tamoxifen, we used Cox multiple-regression analysis that included tumor size, grade, lymph node metastasis, menopausal status, HR status, additional adjuvant chemotherapy. Beside tumor size, methylated RASSF1A serum DNA was strongly associated with poor outcome, with a relative risk for relapse of 5.1 (Table 12A). The only predictor for poor overall survival was RASSF1A serum DNA methylation, with a relative risk for death of 6.9 (Table 12B). To assess which patients might profit from adjuvant tamoxifen treatment and which patients should be offered an alternative therapy to prevent relapse and/or death from breast cancer, we grouped patients into three categories according to RASSF1A DNA methylation in pretherapeutic and one-year-after serum: (i) primary positive that switched to negative after one year, (ii) always negative, (iii) positive after one year, irrespective of primary methylation status. Despite no difference in the follow-up period or any other clinicopathological feature or treatment modality, 0% and 21% of patients relapsed and 5% and 24% of patients died in the “Pos→Neg” and “Finally Pos” groups, respectively (FIG. 5). With regard to survival, no statistically significant difference between the “Pos→Neg” and “Always Neg” groups was observed.

To date there has been no target to assess whether a patient will truly profit from adjuvant therapy or not following tumor removal. The invention now provides a simple tool for indicating “tumor activity” that is non-responsive to a patient's current systemic therapy. To our knowledge no systemic marker for monitoring adjuvant treatment in breast cancer patients has yet been established.

During recent years some studies have reported cell-free DNA in serum/plasma of breast cancer patients at diagnosis (Silva, (1999), loc. cit.; Muller, (2003) loc. cit.; Silva, (2002), Ann. Surg. Oncol. 9, 71-79; Shao, (2001), Clin. Cancer Res. 7, 2222-2227). Although it is evident that DNA circulates freely in the bloodstream of healthy controls or even in cancer patients, the source of this DNA remains enigmatic. Within this paper we demonstrate that RASSF1A DNA methylation is present in nearly all breast cancers and rare in patients with non-neoplastic breast conditions or patients with other invasive cancers, like cervical or prostate cancer. Therefore, serum RASSF1A DNA methylation is a surrogate marker for circulating breast cancer cells and disappearance indicates a response, whereas persistence or reappearance means resistance to adjuvant tamoxifen treatment.

The most common hypothesis concerning the origin of circulating tumor-specific DNA, namely the lysis of circulating cancer cells or micrometastasis shed by the tumor, has turned out to be wrong because there are not enough circulating cells to justify the amount of DNA found in the bloodstream. It thus appears that circulating tumor-specific DNA could be due either to DNA leakage resulting from tumor necrosis or apoptosis or to a new mechanism of active release (Anker, (1999) Cancer Metastasis Rev. 18, 65-73).

RASSF1A methylation has first been described in lung and breast cancer (Dammann, (2000) Nat. Genet. 25, 315-319; Dammann, (2001) Cancer Res. 61, 3105-3109) and is thought to act as a key player in regulating mitosis (Song, (2004) Nat. Cell Biol. 6, 129-137) inducing the stability of mitotic cyclins and timing of mitotic progression. Additionally, RASSF1A localizes to microtubules during interphase and to centrosomes and the spindle during mitosis and the overexpression of RASSF1A-induced stabilization of mitotic cyclins and mitotic arrest at prometaphase (Song, (2004) loc. cit.).

Adjuvant endocrine therapy is one of the keys to improving breast cancer-specific survival. Recently, a prospective, placebo-controlled trial demonstrated beneficial effects of the aromatase inhibitor letrozole, a drug that reduces local production of estradiol, after discontinuation of tamoxifen therapy (Goss, (2003), loc. cit.). Of the 2582 patients treated in the letrozole arm only 29 women profited from this treatment by developing no distant metastases as compared to the placebo group. This means that 100 patients have to be treated in order to prevent distant metastasis in one patient. As aromatase inhibitors are potentially harmful (e.g. osteoporosis) and cause discomfort (e.g. arthralgia, myalgia) to patients as well as economic strain to the health system, tools to identify patients likely to profit from this treatment are acutely needed. Serum RASSF1A DNA methylation is an easy means of detecting patients undergoing adjuvant tamoxifen treatment who need secondary adjuvant therapy. We were able to detect RASSF1A methylation in about 20% of breast cancer patients one year after treatment commencement. It is plausible to speculate that only these patients will benefit from further adjuvant treatment. Using a simple test like RASSF1A DNA methylation in serum after a certain period of adjuvant treatment with anti-estrogens permits detection of those patients who need further treatment with other substances like aromatase inhibitors or alternative therapies. The ability to detect such patients would have a great impact on cost effectiveness and on preventing side-effects in patients otherwise “over-treated” with adjuvant treatment.

### Example 3

### RASSF1A DNA Methylation in Serum is Also an Independent Prognostic Marker in Patients with Breast Cancer Metastasis

It was evaluated whether the number of locations of metastasis, CA153 and DNA methylation status of RASSF1A are prognostic markers in patients with metastasized breast cancer.

### Material and Methods:

RASSF1A DNA methylation in sera (collected before (median: 15 days) or at the time of diagnosis of relapse) of 42 patients (all younger than 60 years of age at the time of relapse) with secondary developed, measurable metastatic breast cancer have been analyzed. DNA isolation, bisulfite modification and MethyLight assay has been performed as described elsewhere.

### Results:

Neither CA153 levels (FIG. 6) nor the number of locations of metastasis (FIG. 7) demonstrated prognostic potential in this group of patients.

RASSF1A DNA methylation in the same serum that has been analyzed for CA153 was a poor prognostic marker (FIG. 8).

The Cox multiple-regression analysis included CA153, number of locations of metastasis and RASSF1A methylation status. Methylated RASSF1A in serum DNA was strongly associated with poor outcome, with a relative risk for death of 3.24 (95% CI: 1.4-7.7; p=0.008). This means that patients who had RASSF1A methylated in their serum had a 3.24 higher risk (independent of all other poor prognostic markers like CA153 or number of sites of metastasis) to die within the observation period, compared to patients with metastatic breast cancer who had no RASSF1A methylated in their serum.

Up to data, beside CT scan, sonography and other imaging methods, the serum tumor marker CA153 is used to monitor efficacy of therapy in patients with metastatic breast cancer. Our data demonstrate that methylation of RASSF1A in the serum outperforms CA153 levels regarding the prognostic value. In view of the data reported above, RASSF1A methylation in the serum also outperforms CA153's potency to predict the response to systemic therapy in patients with metastatic breast cancer.

TABLE 1

| | Genomic sequence | |
|---|---|---|
| | (SEQ ID | Bisulphite sequence |
| Gene | NO.) | (SEQ ID NO.) |
| HIC1 | 1 | 6, 7, 16 and 17 |
| NM_006497 | | |
| HSD17B4 | 2 | 8, 9, 18 and 19 |
| NM_000414 | | |
| APC | 3 | 10, 11, 20 and 21 |
| NM_000038 | | |
| ESR1 | 4 | 12, 13, 22 and 23 |
| NM_000125 | | |
| RASSF1A | 5 | 14, 15, 24 and 25 |
| NM_170715 | | |

TABLE 2
Characteristics of training and test sets.

| | | |
|---|---|---|
| Training Set | Test Set | |
| (N = 24) | (N = 62) | P |

| Characteristics | percent | | Value* |
|---|---|---|---|
| Size of tumour | | | 0.024 |
| T1 | 62.5 | 79 | |
| T2 | 37.5 | 13 | |
| T3 + T4 | 0 | 7 | |
| Histologic type | | | n.s |
| Invasive ductal | 67 | 63 | |
| Invasive lobular | 8 | 13 | |
| Others | 25 | 24 | |
| Tumor grade | | | n.s |
| 1 | 46 | 44 | |
| 2 | 33 | 39 | |
| 3 | 17 | 10 | |
| Lymph node | | | n.s. |
| metastases | | | |
| No | 75 | 65 | |
| Yes | 12.5 | 11 | |
| Unknown | 12.5 | 24 | |
| Menopausal status | | | n.s. |
| Premenopausal | 33 | 16 | |
| Postmenopausal | 67 | 84 | |
| Estrogen-receptor | | | n.s. |
| status | | | |
| Positive | 54 | 40 | |
| Negative | 42 | 45 | |
| Progensteron-receptor | | | n.s. |
| status | | | |
| Positive | 58 | 45 | |
| Negative | 38 | 40 | |
| Hormone-receptor | | | n.s. |
| status | | | |
| Positive | 63 | 50 | |
| Negative | 33 | 36 | |

*P values for the comparison of numbers of patients were calculated by means of the Chi² test. n.s., not significant; Median age: training set (54.2 years; 37.6-83.2), test set (65.7 years; 28.2-86.2), P = 0.052; Follow-up: training set (8.0 years; 1 month to 12.2. years), test set (3.1 years; 1 month to 11 years) P < 0.001.
Tumour grade was unknown in six cases.
Hormone-receptor status was unknown in ten cases.
Tumour size was unknown in one case.

TABLE 3
Frequency of methylated serum DNA in the gene evaluation set.

| | | Primary Breast | Recurrent Breast |
|---|---|---|---|
| | Healthy Controls | Cancer | Cancer |
| | (N = 10) | (N = 26) | (N = 10) |
| Gene | percent positive | | |
| ESR1 | 0 | 27 | 70 |
| APC | 0 | 23 | 80 |
| HSD17B4 | 0 | 12 | 30 |
| CDH13 | 0 | 8 | 40 |
| ESR2 | 0 | 4 | 20 |
| MGMT | 0 | 4 | 10 |
| SYK | 0 | 4 | 10 |
| HIC1 | 10 | 39 | 90 |
| RASSF1A | 10 | 23 | 80 |
| GSTP1 | 10 | 12 | 60 |
| MYOD1 | 20 | 27 | 80 |
| CDH1 | 20 | 20 | 90 |
| PTGS2 | 30 | 39 | 100 |
| PGR | 30 | 46 | 80 |
| CALCA | 40 | 50 | 60 |
| HLAG | 60 | 69 | 100 |
| BLT1 | 60 | 85 | 100 |
| ARHI | 100 | 100 | 100 |
| MLLT7 | 100 | 100 | 100 |
| TFF1 | 100 | 100 | 100 |
| SOCS2 | 0 | 0 | 40 |
| SOCS1 | 0 | 0 | 30 |
| TERT | 0 | 0 | 30 |
| DAPK1 | 0 | 0 | 30 |
| TIMP3 | 0 | 0 | 20 |
| BRCA1 | 0 | 0 | 20 |
| GSTM3 | 0 | 0 | 20 |
| MT3 | 0 | 0 | 20 |
| TWIST | 0 | 0 | 10 |
| MLH1 | 0 | 0 | 10 |
| CYP1B1 | 0 | 0 | 10 |
| TITF1 | 0 | 0 | 10 |
| FGF18 | 0 | 0 | 10 |
| CDKN2A | n.d. | n.d. | 0 |
| HSPA2 | n.d. | n.d. | 0 |
| PPP1R13B | n.d. | n.d. | 0 |
| TP53BP2 | n.d. | n.d. | 0 |
| REV3L | n.d. | n.d. | 0 |
| IGFB2 | n.d. | n.d. | 0 |

n.d., not done

TABLE 4
Univariate analysis of methylation status in training and test sets.

| | Training Set | Test Set |
|---|---|---|
| | (N = 24) | (N = 62) |
| Genes | P Value | P Value |
| ESR1 | 0.018 | 0.555 |
| APC | 0.002 | 0.307 |
| HSD17B4 | 0.056 | |
| HIC1 | 0.796 | |
| RASSF1A | 0.042 | 0.014 |
| RASSF1A/APC | <0.001 | 0.007 |
| ESR1/APC | 0.001 | 0.951 |
| ESR1/RASSF1A | 0.032 | 0.138 |

*P values for each variable were calculated by means of the log rank test.

TABLE 5
Frequency of methylated genes according to
clinicopathological features.

| | | | | | RASSF1A |
|---|---|---|---|---|---|
| | | | | | and/or |
| | No. of | ESR1 | APC | RASSF1A | APC |
| Characteristics | Patients | % positive | | | |
| Size of tumour | | | | | |
| T1 | 64 | 14 | 11 | 9 | 19 |
| T2 | 17 | 12 | 12 | 19 | 31 |
| T3 + T4 | 4 | 75 | 25 | 50 | 50 |
| Histologic type | | | | | |
| Invasive ductal | 55 | 18 | 15 | 11 | 22 |
| Invasive lobular | 10 | 20 | 0 | 30 | 30 |
| Others | 21 | 10 | 10 | 10 | 20 |
| Tumor grade | | | | | |
| 1 | 38 | 11 | 11 | 13 | 21 |
| 2 | 32 | 19 | 16 | 16 | 31 |
| 3 | 10 | 30 | 10 | 11 | 11 |
| Lymph node metastases | | | | | |
| No | 58 | 12 | 9 | 9 | 18 |
| Yes | 10 | 20 | 30 | 20 | 40 |
| Unknown | 18 | 28 | 11 | 22 | 28 |
| Menopausal status | | | | | |
| Premenopausal | 18 | 28 | 11 | 11 | 22 |
| Postmenopausal | 68 | 13 | 12 | 13 | 22 |
| Estrogen-receptor | | | | | |
| Positive | 38 | 16 | 11 | 16 | 21 |
| Negative | 38 | 16 | 16 | 11 | 27 |
| Progensterone- | | | | | |
| receptor | | | | | |
| Positive | 42 | 14 | 5 | 14 | 18 |
| Negative | 34 | 18 | 24 | 12 | 33 |
| Hormone-receptor | | | | | |
| status | | | | | |
| Positive | 46 | 15 | 9 | 15 | 20 |
| Negative | 30 | 17 | 20 | 10 | 31 |

Tumour grade was unknown in six cases.
Hormone-receptor status was unknown in ten cases.
Tumour size was unknown in one case.
DNA methylation of RASSF1A for one case was missing.
Chi² Pearson: Tumour size - ESR1 (P = 0.005); Tumour size - RASSF1A (P = 0.049); Progesterone-receptor - APC (P = 0.036); Median age - RASSF1A methylated (79.0 yrs.; 49.6 to 86.2), RASSF1A unmethylated (59.4 yrs.; 28.2 to 82.3.) P = 0.009

TABLE 6
Results of univariate analysis.

| | No. of Patients | Relative | |
|---|---|---|---|
| | Who Died/Total | Risk of Death | P |
| Variable | No. | (95% CI) | Value |
| Size of tumour | | | 0.018 |
| T1 | 10/64 | | |
| T2 | 5/17 | 2.2 (0.6-7.8) | |
| T3 + T4 | 2/4 | 5.4 (0.7-42.9) | |
| Histologic type | | | 0.296 |
| Invasive ductal | 13/55 | | |
| Invasive lobular | 1/10 | 0.4 (0-3.1) | |
| Others | 3/21 | 0.5 (0.1-2.1) | |
| Tumor grade | | | 0.310 |
| 1 | 6/38 | | |
| 2 | 9/32 | 2.1 (0.7-6.7) | |
| 3 | 2/10 | 1.3 (0.2-7.9) | |
| Lymph node metastases | | | 0.005 |
| No | 7/58 | | |
| Yes | 5/10 | 7.3 (1.7-31.7) | |
| Unknown | 5/18 | 2.8 (0.8-10.3) | |
| Menopausal status | | | 0.062 |
| Premenopausal | 1/18 | | |
| Postmenopausal | 16/68 | 5.2 (0.6-42.4) | |
| Estrogen-receptor | | | 0.369 |
| Positive | 10/38 | 1.9 (0.6-5.9) | |
| Negative | 6/38 | | |
| Progensterone-receptor | | | 0.766 |
| Positive | 9/42 | 1.1 (0.3-3.2) | |
| Negative | 7/34 | | |
| Hormone-receptor | | | 0.799 |
| status | | | |
| Positive | 10/46 | 1.1 (0.4-3.5) | |
| Negative | 6/30 | | |
| ESR1 methylation | | | 0.370 |
| Unmethylated | 13/72 | | |
| Methylated | 4/14 | 1.8 (0.5-6.7) | |
| APC methylation | | | 0.001 |
| Unmethylated | 12/76 | | |
| Methylated | 5/10 | 5.3 (1.3-21.3) | |
| RASSF1A methylation | | | 0.001 |
| Unmethylated | 11/74 | | |
| Methylated | 6/11 | 6.9 (1.8-26.5) | |
| RASSF1/APC methylation | | | <0.001 |
| Unmethylated | 7/66 | | |
| Methylated | 10/19 | 9.5 (2.9-31.4) | |

TABLE 7
Multivariate Analysis.

| | Relative Risk of Death | P |
|---|---|---|
| Variable | (95% CI) | Value |
| Size of tumour | | 0.19 |
| T2 (vs. T1) | 2.7 (0.8-9.3) | |
| T3 + T4 (vs. T1) | 2.9 (0.4-20.5) | |
| Lymph node metastases | | 0.039 |
| Yes (vs. no lymph node metastases) | 3.9 (1.1-13.9) | |
| Unknown (vs. no lymph node metastases) | 5.2 (1.2-22.4) | |
| Age | 1.0 (1.0-1.1) | 0.06 |
| RASSF1A and/or APC methylated | 5.7 (1.9-16.9) | 0.002 |
| (vs. unmethylated) | | |

TABLE 8
Sequences of the primers and probes

| HUGO Gene | | | |
|---|---|---|---|
| Nomenclature | Forward Primer Sequence | Reverse Primer Sequence | Probe Oligo Sequence |
| ACTB | TGGTGATGGAGGAGGTTTAGTAAGT | AACCAATAAAACCTACTCCTCCCTAA | 6FAM-ACCACCACCCAACACACAA |
| | | | TAACAAACACA-BHQ-1 |
| APC | GAACCAAAACGCTCCCCAT | TTATATGTCGGTTACGTGCGTTTATAT | 6FAM-CCCGTCGAAAACCCGCCGA |
| | | | TTA-BHQ-1 |
| ARHI | GCGTAAGCGGAATTTATGTTTGT | CCGCGATTTTATATTCCGACTT | 6FAM-CGCACAAAAACGAAATACG |
| | | | AAAACGCAAA-BHQ-1 |
| BLT1 | GCGTTGGTTTTATCGGAAGG | AAACCGTAATTCCCGCTCG | 6FAM-GACTCCGCCCAACTTCGCC |
| | | | AAAA-BHQ-1 |
| BRCA1 | GAGAGGTTGTTGTTTAGCGGTAGTT | CGCGCAATCGCAATTTTAAT | 6FAM-CCGCGCTTTTCCGTTACCA |
| | | | CGA-BHQ-1 |
| CALCA | GTTTTGGAAGTATGAGGGTGACG | TTCCCGCCGCTATAAATCG | 6FAM-ATTCCGCCAATACACAACA |
| | | | ACCAATAAACG-BHQ-1 |
| CDH1 | AATTTTAGGTTAGAGGGTTATCGCGT | TCCCCAAAACGAAACTAACGAC | 6FAM-CGCCCACCCGACCTCGCA |
| | | | T-BHQ-1 |
| CDH13 | AATTTCGTTCGTTTTGTGCGT | CTACCCGTACCGAACGATCC | 6FAM-AACGCAAAACGCGCCCGA |
| | | | CA-BHQ-1 |
| CDKN2A | TGGAGTTTTCGGTTGATTGGTT | AACAACGCCCGCACCTCCT | 6FAM-ACCCGACCCCGAACCGC |
| | | | G-BHQ-1 |
| CYP1B1 | GTGCGTTTGGACGGGAGTT | AACGCGACCTAACAAAACGAA | 6FAM-CGCCGCACACCAAACCGCT |
| | | | T-BHQ-1 |
| DAPK1 | TCGTCGTCGTTTCGGTTAGTT | TCCCTCCGAAACGCTATCG | 6FAM-CGACCATAAACGCCAACGC |
| | | | CG-BHQ-1 |
| ESR1 | GGCGTTCGTTTTGGGATTG | GCCGACACGCGAACTCTAA, | 6FAM-CGATAAAACCGAACGACCC |
| | | | GACGA-BHQ-1 |
| ESR2 | TTTGAAATTTGTAGGGCGAAGAGTAG | ACCCGTCGCAACTCGAATAA | 6FAM-CCGACCCAACGCTCGCCG- |
| | | | BHQ-1 |
| FGF18 | ATCTCCTCCTCCGCGTCTCT | TCGCGCGTAGAAAACGTTT | 6FAM-CGACCGTACGCATCGCCG |
| | | | C-BHQ-1 |
| GSTM3 | GCG CGA ACG CCC TAA CT | AAC GTC GGT ATT AGT CGC GTT T | 6FAM-CCC CGT TCT CCG TCC |
| | | | CTT ACC TCC-BHQ-1 |
| GSTP1 | GTCGGCGTCGTGATTTAGTATTG | AAACTACGACGACGAAACTCCAA | 6FAM-AAACCTCGCGACCTCCGAA |
| | | | CCTTATAAAA-BHQ-1 |
| HIC1 | GTTAGGCGGTTAGGGCGTC | CCGAACGCCTCCATCGTAT | 6FAM-CAACATCGTCTACCCAACA |
| | | | CACTCTCCTACG-BHQ-1 |
| HLA-G | CAC CCC CAT ATA CGC GCT AA | GGT CGT TAC GTT TCG GGT AGT TTA | 6FAM-CGC GCT CAC ACG CTC |
| | | | AAA AAC CT-BHQ1 |
| HSD17B4 | TATCGTTGAGGTTCGACGGG | TCCAACCTTCGCATACTCACC | 6FAM-CCCGCGCCGATAACCAATA |
| | CAC GAA CAC TAC CAA CAA CTC | | CCA-BHQ-1 |
| HSPA2 | AAC T | GGG AGC GGA TTG GGT TTG | 6FAM-CCG CGC CCA ATT CCC |
| | | | GAT TCT-BHQ1 |
| IGFBP2 | CTC GCG CCG ACA AAT AAA TAC GT | CGG GAA GAG TAG GGA ATT TTT AGA | 6FAM-ACG CCC GCT CGC CCA |
| | | | CCT-BHQ1 |
| MGMT | GCGTTTCGACGTTCGTAGGT | CACTCTTCCGAAAACGAAACG | 6FAM-CGCAAACGATACGCACCGC |
| | | | GA-BHQ-1 |
| MLH1 | AGGAAGAGCGGATAGCGATTT | TCTTCGTCCCTCCCCTAAAACG | 6FAM-CCCGCTACCTAAAAAAATA |
| | | | TACGCTTACGCG-BHQ-1 |
| MLLT7 | CCT CAC GAT ACC TCC CCT CAA | TTA GGG ATT AGC GTT TTG GGA TT | 6FAM-AAA CAC ATT CCT ACC |
| | | | AAT CTT CAA AAA ATC GCG- |
| | | | BHQ-1 |
| MT3 | CGA TAA ACG AAC TTC TCC AAA | GCG CGG TGC GTA GGG | 6FAM-AAA CGC GCG ACT TAA |
| | CAA | | CTA ATA ACA ACA AAT AAC |
| | | | GA-BHQ-1 |
| MYOD1 | GAGCGCGCGTAGTTAGCG | TCCGACACGCCCTTTCC | 6FAM-CTCCAACACCCGACTACTA |
| | | | TATCCGCGAAA-BHQ-1 |
| PGR | TTATAATTCGAGGCGGTTAGTGTTT | TCGAACTTCTACTAACTCCGTACTACGA | 6FAM-ATCATCTCCGAAAATCTCA |
| | | | AATCCCAATAATACG-BHQ-1 |
| PPP1R13B | CCT CAC CCA CCG ACA TCA TC | TCG GAG CGG TGG GTA TAG TTC | 6FAM-AAA AAT CCG CGA CGC |
| | | | CCT CGA-BHQ-1 |
| PTGS2 | CGGAAGCGTTCGGGTAAAG | AATTCCACCGCCCCAAAC | 6FAM-TTTCCGCCAAATATCTTTT |
| | | | CTTCTTCGCA-BHQ-1 |
| RASSF1A | ATTGAGTTGCGGGAGTTGGT | ACACGCTCCAACCGAATACG | 6FAM-CCCTTCCCAACGCGCCCA- |
| | | | BHQ-1 |
| REV3L | CGA ACG CAA CCG ACC CT | TAT TTT TCG TAT CGT TTT CGG GTT A | 6FAM-CTC AAA TAA CGC CGC |
| | | | GAC TCC GC-BHQ-1 |
| SOCS1 | GCGTCGAGTTCGTGGGTATTT | CCGAAACCATCTTCACGCTAA | 6FAM-ACAATTCCGCTAACGACTA |
| | | | TCGCGCA-BHQ-1 |
| SOCS2 | TCC CTT CCC CGC CAT T | TTG TTT TTG TCG CGG TGA TTT | 6FAM-CCG AAA AAC TCA AAA |
| | | | CAC CGC AAA ATC AT-BHQ-1 |
| SYK | GGGCGCGATATTGGGAG | GCGACTCTTCCTCATTTTAAACAAC | 6FAM-CCTTAACGCGCCCGAACAA |
| | | | ACG-BHQ-1 |
| TERT | GGATTCGCGGGTATAGACGTT | CGAAATCCGCGCGAAA | 6FAM-CCCAATCCCTCCGCCACGT |
| | | | AAAA-BHQ-1 |
| TFF1 | TAAGGTTACGGTGGTTATTTCGTGA | ACCTTAATCCAAATCCTACTCATATCTA | 6FAM-CCCTCCCGCCAAAATAAAT |
| | | AAA | ACTATACTCACTACAAAA-BHQ-1 |
| TIMP3 | GCGTCGGAGGTTAAGGTTGTT | CTCTCCAAAATTACCGTACGCG | 6FAM-AACTCGCTCGCCCGCCGA |
| | | | A-BHQ-1 |
| TITF1 | CGA AAT AAA CCG AAT CCT CCT | TGT TTT GTT GTT TTA GCG TTT ACG T | 6FAM-CTC GCG TTT ATT TTA |
| | TAA | | ACC CGA CGC CA-BHQ-1 |
| TP53BP2 | ACC CCC TAA CGC GAC TTT ATC | GTT CGA TTC GGG ATT AGT TGG T | 6FAM-CGC TCG TAA CGA TCG |
| | | | AAA CTC CCT CCT-BHQ-1 |
| TWIST | GTAGCGCGGCGAACGT | AAACGCAACGAATCATAACCAAC | 6FAM-CCAACGCACCCAATCGCTA |
| | | | AACGA-BHQ-1 |

TABLE 9
Characteristics of breast cancer patients included
in the analysis.

| | Patients | |
|---|---|---|
| | (N = 148) | |
| Characteristic | no. (%) | |
| Size of tumor | | |
| T1 | 92 | (62.2) |
| T2 | 42 | (28.4) |
| T3 + T4 | 14 | (9.5) |
| Histologic type | | |
| Invasive ductal | 110 | (74.3) |
| Invasive lobular | 19 | (12.8) |
| Others | 19 | (12.8) |
| Tumor grade | | |
| I | 47 | (31.8) |
| II | 83 | (56.1) |
| III | 14 | (9.5) |
| unknown | 4 | (2.7) |
| Lymph node metastases | | |
| Negative | 88 | (59.5) |
| one to three nodes positive | 31 | (20.9) |
| more than three nodes positive | 20 | (13.5) |
| unknown | 9 | (6.1) |
| Menopausal status | | |
| Premenopausal | 30 | (20.3) |
| Postmenopausal | 118 | (79.7) |
| Estrogen-receptor status | | |
| Positive | 129 | (87.2) |
| Negative | 18 | (12.2) |
| Unknown | 1 | (0.7) |
| Progesterone-receptor status | | |
| Positive | 123 | (83.1) |
| Negative | 25 | (16.9) |
| Hormone-receptor status | | |
| Positive | 141 | (95.3) |
| Negative | 7 | (4.7) |
| Adjuvant radiation therapy | | |
| No | 48 | (32.4) |
| Yes | 100 | (76.6) |
| Additional chemotherapy | | |
| No | 97 | (65.5) |
| Yes | 51 | (34.5) |
| Type of surgery | | |
| BE | 81 | (54.7) |
| ME | 67 | (45.3) |

TABLE 10
Characteristics of patients according to RASSF1A methylation
status in pretherapeutic and one-year-after serum samples
(without and with parenthesis, respectively).

| | Unmethylated | Methylated | |
|---|---|---|---|
| | N = 119 (115) | N = 29 (33) | Pearson's Chi |
| Characteristic | no. of patients | | square Test |
| Size of tumor | | | 0.05 (0.55) |
| T1 | 79 (73) | 13 (19) | |
| T2/3/4 | 40 (42) | 16 (14) | |
| Tumor grade | | | 0.18 (0.40) |
| I | 41 (34) | 6 (13) | |
| II/III | 75 (77) | 22 (20) | |
| Lymph node metastases | | | 0.82 (0.53) |
| Negative | 73 (70) | 15 (18) | |
| Positive | 41 (38) | 10 (13) | |
| Menopausal status | | | 0.01 (0.09) |
| Premenopausal | 29 (27) | 1 (3) | |
| Postmenopausal | 90 (88) | 28 (33) | |
| Hormone-receptor status | | | 1.00 (0.35) |
| Positive | 113 (108) | 28 (33) | |
| Negative | 6 (7) | 1 (0) | |

TABLE 11A
Results of univariate analysis for relapse-free survival.

| | No. of | Relative Risk | |
|---|---|---|---|
| | patients who | of relapse | |
| Variable | relapsed/total No. | (95% CI) | P Value |
| Size of tumor | | | <0.001 |
| T1 | 2/92 | | |
| T2/3/4 | 11/56 | 10.0 (2.2-45.3) | |
| Tumor grade | | | 0.04 |
| I | 2/47 | | |
| II/III | 11/97 | 4.3 (0.9-19.7) | |
| Lymph node metastases | | | 0.003 |
| Negative | 3/88 | | |
| Positive | 10/51 | 5.8 (1.6-21.0) | |
| Menopausal status | | | 0.89 |
| Premenopausal | 3/30 | | |
| Postmenopausal | 10/118 | 1.1 (0.3-4.0) | |
| Hormone-receptor status | | | 0.68 |
| Negative | 1/7 | | |
| Positive | 12/141 | 0.7 (0.1-5.1) | |
| Pretherapeutic RASSF1A | | | 0.53 |
| methylation | | | |
| Negative | 10/119 | | |
| Positive | 3/29 | 1.5 (0.4-5.8) | |
| “One-year-after” | | | 0.005 |
| RASSF1A methylation | | | |
| Negative | 6/115 | | |
| Positive | 7/33 | 4.2 (1.4-12.5) | |

TABLE 11B
Results of univariate analysis for overall survival.

| | No. of | | |
|---|---|---|---|
| | patients who | Relative Risk of | |
| Variable | died/Total No. | Death (95% CI) | P Value |
| Size of tumor | | | 0.02 |
| T1 | 5/92 | | |
| T2/3/4 | 10/56 | 3.4 (1.2-10.0) | |
| Tumor grade | | | 0.06 |
| I | 3/47 | | |
| II/III | 12/97 | 3.2 (0.9-11.3) | |
| Lymph node metastases | | | 0.03 |
| Negative | 5/88 | | |
| Positive | 9/51 | 3.2 (1.1-9.7) | |
| Menopausal status | | | 0.34 |
| Premenopausal | 2/30 | | |
| Postmenopausal | 13/118 | 2.0 (0.5-9.2) | |
| Hormone-receptor status | | | 0.72 |
| Negative | 1/7 | | |
| Positive | 14/141 | 0.7 (0.1-5.2) | |
| Pretherapeutic RASSF1A | | | 0.28 |
| methylation | | | |
| Negative | 11/119 | | |
| Positive | 4/29 | 1.9 (0.6-6.1) | |
| “One-year-after” RASSF1A | | | 0.002 |
| methylation | | | |
| Negative | 7/115 | | |
| Positive | 8/33 | 4.7 (1.6-13.6) | |

TABLE 12A
Results of multivariate analysis for
relapse-free survival.

| | Relative Risk of | |
|---|---|---|
| Variable | Relapse (95% CI) | P VALUE |
| Size of tumor | | |
| T2/T3/T4 vs T1 | 4.7 (1.0-24.4) | 0.05 |
| Tumor grade | | |
| II/III vs I | 3.6 (0.6-20.2) | 0.15 |
| Lymph node metastases | | |
| Positive vs Negative | 2.3 (0.5-10.3) | 0.27 |
| Menopausal status | | |
| Postmenopausal vs Premenopausal | 1.7 (0.3-11.1) | 0.59 |
| Hormone-receptor status | | |
| Positive vs Negative | 0.5 (0.04-6.0) | 0.57 |
| Additional chemotherapy | | |
| Yes vs No | 3.1 (0.5-19.3) | 0.22 |
| “One-year-after” RASSF1A | | |
| methylation | | |
| Positive vs Negative | 5.1 (1.3-19.8) | 0.02 |

TABLE 12B
Results of multivariate analysis for overall survival.

| | Relative Risk of | |
|---|---|---|
| Variable | Death (95% CI) | P Value |
| Size of tumor | | |
| T2/T3/T4 vs T1 | 2.8 (0.7-10.9) | 0.14 |
| Tumor grade | | |
| II/III vs I | 3.8 (0.8-16.9) | 0.09 |
| Lymph node metastases | | |
| Positive vs Negative | 2.9 (0.7-12.1) | 0.14 |
| Menopausal status | | |
| Postmenopausal vs | 2.8 (0.4-22.1) | 0.30 |
| Premenopausal | | |
| Hormone-receptor status | | |
| Positive vs Negative | 0.3 (0.02-4.2) | 0.37 |
| Additional chemotherapy | | |
| Yes vs No | 0.7 (0.2-3.3) | 0.70 |
| “One-year-after” RASSF1A | | |
| methylation | | |
| Positive vs Negative | 6.9 (1.9-25.9) | 0.004 |

1. A method for determining the prognosis of a subject with a cell proliferative disorder of the breast tissues, said method comprising analysing the methylation pattern of a target nucleic acid comprising one or a combination of the genes taken from the group consisting of ESR1, APC, HSD174B4, HIC1 and RASSF1A and/or their regulatory regions by contacting at least one of said target nucleic acids in a biological sample obtained from said subject with at least one reagent, or series of reagents that distinguishes between methylated and non-methylated CpG dinucleotides.

2. A method for selecting a treatment and/or for monitoring a treatment of a cell proliferative disorder of the breast tissues, said method comprising:
a) determining the prognosis of a subject according to claim 1, and
b) selecting a suitable treatment according to said prognosis and/or monitoring the treatment success according to said prognosis.

3. The method of claim 2, wherein said suitable treatment is a hormonal/antihormonal therapy, a chemotherapy and/or an adjuvant therapy.

4. The method of claim 3, wherein said suitable treatment is a hormonal/antihormonal therapy and wherein the determination of said prognosis comprises the analysis of the methylation pattern of a target nucleic acid comprising the RASSF1A gene and/or its regulatory region(s).

5. The method of claim 3 or 4, wherein said hormonal/antihormonal therapy comprises a tamoxifen therapy.

6. The method of claim 5, wherein persistence, increase, appearance or re-appearance of RASSF1A methylation indicates a resistance to tamoxifen treatment and/or wherein a decrease or disappearance of RASSF1A methylation is indicative for a response to tamoxifen treatment.

7. A method for determining the phenotype of a subject with a breast cell proliferative disorder comprising
a) obtaining a biological sample containing genomic DNA from said subject,
b) analysing the methylation pattern of one or more target nucleic acids comprising one or a combination of the genes taken from the group consisting of ESR1, APC, HSD174B4, HIC1 and RASSF1A and/or their regulatory regions by contacting at least one of said target nucleic acids in the biological sample obtained from said subject with at least one reagent, or series of reagents that distinguishes between methylated and non-methylated CpG dinucleotides, and
c) determining the phenotype of the individual by comparison to two known phenotypes, a first phenotype characterised by hypermethylation of the target nucleic acid and poor prognosis as relative to a second phenotype characterised by hypomethylation of the analysed target nucleic acid and positive prognosis.

8. A method according to claims 1 to 3, 7 and 8 wherein said prognosis is the life expectancy of said subject or wherein said prognosis is the treatment success of a cell proliferative disorder of the breast tissues.

9. A method according to any one of claims 1 to 3, 7 and 8 wherein said target nucleic acid comprises the gene APC and/or its regulatory regions.

10. A method according to any one of claims 1 to 8 wherein said target nucleic acid comprises the gene RASSF1A and/or its regulatory regions.

11. A method according to any one of claims 1 to 8 wherein said target nucleic acids comprise the genes APC and RASSF1A and/or their regulatory regions.

12. A method according to any one of claims 1 to 11, wherein said target nucleic acid or acids comprise essentially one or more sequences from the group consisting of SEQ ID NOs: 1 to 5 and sequences complementary thereto.

13. A method according to claim 9 wherein the sequence of said target nucleic acid is or comprises the nucleic acid molecule of SEQ ID NO: 3 or a fragment thereof.

14. A method according to claim 10 wherein the sequence of said target nucleic acid is or comprises the nucleic acid molecule of SEQ ID NO: 5 or a fragment thereof.

15. A method according to claim 11, wherein said target nucleic acid or acids is or comprises the nucleic acid molecule as shown in SEQ ID NOs: 3 and 5 or a fragment of said nucleic acid molecules.

16. A method according to any one of claims 1 to 15, wherein said cell proliferative disorder of the breast tissue is selected from the group consisting of ductal carcinoma in situ, lobular carcinoma, colloid carcinoma, tubular carcinoma, medullary carcinoma, metaplastic carcinoma, intraductal carcinoma in situ, lobular carcinoma in situ and papillary carcinoma in situ.

17. A method according to any one of claims 1 to 16, wherein said biological sample is a blood sample, serum or NAF (nipple aspirate fluid).

18. A nucleic acid molecule consisting essentially of a sequence at least 18 bases in length according to one of the sequences taken from the group consisting of SEQ ID NOs: 6 to 25 and sequences complementary thereto.

19. An oligomer, in particular an oligonucleotide or peptide nucleic acid (PNA)-oligomer, said oligomer consisting essentially of at least one base sequence having a length of at least 10 nucleotides which hybridises to or is identical to one of the nucleic acid sequences according to SEQ ID NOs: 6 to 25.

20. The oligomer as recited in any one of claims 18 or 19, wherein the base sequence includes at least one CpG dinucleotide.

21. A set of oligomers, comprising at least two oligomers according to any of claims 18 or 19.

22. A set of oligonucleotides as recited in claim 21, characterised in that at least one oligonucleotide is bound to a solid phase.

23. A set of at least two oligonucleotides as recited in any of claims 19 or 20, which is used as primer oligonucleotides for the amplification of nucleic acid sequences comprising one of SEQ ID NOs: 6 to 25 and sequences complementary thereto.

24. Use of a set of oligonucleotides comprising at least two of the oligomers according to any one of claims 21 to 23 for detecting the cytosine methylation state and/or single nucleotide polymorphisms (SNPs) within the sequences taken from the group SEQ ID NOs: 1 to 5 and sequences complementary thereto.

25. A method for manufacturing an arrangement of different oligomers (array) fixed to a carrier material for predicting the responsiveness of a subject with a cell proliferative disorder of the breast tissues by analysis of the methylation state of any of the CpG dinucleotides of the group SEQ ID NOs 1 to 5 wherein at least one oligomer according to any of the claims 19 or 20 is coupled to a solid phase.

26. An arrangement of different oligomers (array) obtainable according to claim 25.

27. An array of different oligonucleotide- and/or PNA-oligomer sequences as recited in claim 26, characterised in that said oligonucleotides are arranged on a plane solid phase in the form of a rectangular or hexagonal lattice.

28. The array as recited in any of the claims 26 or 27, characterised in that the solid phase surface is composed of silicon, glass, polystyrene, aluminium, steel, iron, copper, nickel, silver, or gold.

29. A DNA- and/or PNA-array for predicting breast cell proliferative disorders' response by analysis of the methylation state of any of the CpG dinucleotides of the group SEQ ID NOs: 1 to 5 comprising at least one nucleic acid according to any of the claims 19 to 23.

30. A method according to any one of claims 1 to 3, 7 and 8 comprising the following steps:
a) obtaining a biological sample containing genomic DNA,
b) extracting the genomic DNA,
c) converting cytosine bases in the genomic DNA sample which are unmethylated at the 5-position, to uracil or another base which is dissimilar to cytosine in terms of base pairing behaviour,
d) amplifying at least one fragment of the pretreated genomic DNA, wherein said fragments comprise one or more sequences selected from the group consisting of SEQ ID NOs: 6 to 25 and sequences complementary thereto, and
e) determining the methylation status of one or more genomic CpG dinucleotides by analysis of the amplificate nucleic acids.

31. A method according to any one of claims 3 to 6 comprising the following steps:
a) obtaining a biological sample containing genomic DNA,
b) extracting the genomic DNA,
c) converting cytosine bases in the genomic DNA sample which are unmethylated at the 5-position, to uracil or another base which is dissimilar to cytosine in terms of base pairing behaviour,
d) amplifying at least one fragment of the pretreated genomic DNA, wherein said fragments comprise one or more sequences selected from the group consisting of SEQ D NOs: 14, 15, 24 and 25 and sequences complementary thereto, and
e) determining the methylation status of one or more genomic CpG dinucleotides by analysis of the amplificate nucleic acids.

32. The method as recited in claims 30 or 31, characterised in that step e) is carried out by means of hybridisation of at least one oligonucleotide according to claims 19 or 20.

33. The method as recited in claims 30 or 31, characterised in that step e) is carried out by means of hybridisation of at least one oligonucleotide according to claims 19 or 20 and extension of said hybridised oligonucleotide(s) by at least one nucleotide base.

34. The method as recited in claims 30 or 31, characterised in that step e) is carried out by means of sequencing.

35. The method as recited in claims 30 or 31, characterised in that step d) is carried out using methylation specific primers.

36. The method as recited in claim 30, further comprising in step d) the use of at least one nucleic acid molecule or peptide nucleic acid molecule comprising in each case a contiguous sequence at least 9 nucleotides in length that is complementary to, or hybridises under moderately stringent or stringent conditions to a sequence selected from the group consisting of SEQ ID NOs: 6 to 25, and complements thereof, wherein said nucleic acid molecule or peptide nucleic acid molecule suppresses amplification of the nucleic acid to which it is hybridised.

37. The method as recited in claim 31, further comprising in step d) the use of at least one nucleic acid molecule or peptide nucleic acid molecule comprising in each case a contiguous sequence at least 9 nucleotides in length that is complementary to, or hybridises under moderately stringent or stringent conditions to a sequence selected from the group consisting of SEQ ID NOs: 14, 15, 24 and 25, and complements thereof, wherein said nucleic acid molecule or peptide nucleic acid molecule suppresses amplification of the nucleic acid to which it is hybridised.

38. The method as recited in claims 30 or 31, characterised in that step e) is carried out by means of a combination of at least two of the methods described in claims 32 to 37.

39. The method as recited in claims 30 or 31, characterised in that the treatment is carried out by means of a solution of a bisulfite, hydrogen sulfite or disulfite.

40. A method according to any one of claims 1 to 16 comprising the following steps:
a) obtaining a biological sample containing genomic DNA,
b) extracting the genomic DNA,
c) digesting the genomic DNA comprising one or more of the sequences from the group consisting of SEQ ID NOs: 1 to 5 and sequences complementary thereto with one or more methylation sensitive restriction enzymes, and
d) determining of the DNA fragments generated in the digest of step c).

41. A method according to claim 40, wherein the DNA digest is amplified prior to step d).

42. The method as recited in any one of claims 30 to 39 and 41, characterised in that more than six different fragments having a length of 100-200 base pairs are amplified.

43. The method as recited in any one of claims 30 to 39, 41 and 42, characterised in that the amplification of several DNA segments is carried out in one reaction vessel.

44. The method as recited in any one of claims 30 to 39, 41 to 43, characterised in that the polymerase is a heat-resistant DNA polymerase.

45. The method as recited in any one of claims 30 to 39, 41 to 44, characterised in that the amplification is carried out by means of the polymerase chain reaction (PCR).

46. The method as recited in any one of claims 30 to 39 and 41 to 45, characterised in that the amplificates carry detectable labels.

47. The method according to claim 46, wherein said labels are fluorescence labels, radionuclides and/or detachable molecule fragments having a typical mass which can be detected in a mass spectrometer.

48. The method as recited in any one of claims 30 to 39 and 41 to 45, characterised in that the amplificates or fragments of the amplificates are detected in the mass spectrometer.

49. The method as recited in any one of the claims 47 and 48, characterised in that the produced fragments have a single positive or negative net charge for better detectability in the mass spectrometer.

50. The method as recited in any one of claims 47 and 48, characterised in that detection is carried out and visualised by means of matrix assisted laser desorption/ionisation mass spectrometry (MALDI) or using electron spray mass spectrometry (ESI).

51. The method as recited in any one of the claims 1 to 16 or any one of the claims 30 to 50, characterised in that the genomic DNA is obtained from cells or cellular components which contain DNA or sources of DNA comprising, for example, cell lines, histological slides, biopsies, tissue embedded in paraffin, breast tissues, blood, plasma, lymphatic fluid, lymphatic tissue, duct cells, ductal lavage fluid, nipple aspiration fluid and combinations thereof.

52. The method as recited in any one of the claims 1 to 16 or any one of the claims 30 to 50, characterised in that said biological sample is or is derived from cell lines, histological slides, biopsies, tissue embedded in paraffin, breast tissues, blood, plasma, lymphatic fluid, lymphatic tissue, duct cells, ductal lavage fluid, nipple aspiration fluid and combinations thereof.

53. A kit comprising a bisulfite (=disulfite, hydrogen sulfite) reagent as well as oligonucleotides, PNA-oligomers and/or sets of oligomers or oligonucleotides according to any one of the claims 19 to 23.

54. A kit according to claim 53, further comprising standard reagents for performing a methylation assay from the group consisting of MS-SNuPE, MSP, Methyl light, Heavy Methyl, nucleic acid sequencing and combinations thereof.

55. The use of a method according to any one of claims 1 to 17 and 30 to 51, a nucleic acid according to claim 18, of an oligonucleotide or PNA-oligomer or a set thereof according to any one of claims 19 to 23, of a kit according to claim 53 or 54, of an arrangement or an array according to any one of claims 26 to 29 or of a method of manufacturing an array according to claim 25 in the prognosis, diagnosis, treatment, characterisation, classification and/or differentiation of breast cell proliferative disorders.

56. The use of claim 55, wherein said treatment is a hormonal/antihormonal treatment.

57. The use of claim 56, wherein said hormonal/antihormonal treatment is a tamoxifen treatment.
